(19) United States
(12) Patent Application Publication
(10) Pub. No.: US 2009/0035796 A1
(43) Pub. Date: Feb. 5, 2009

(54) Enzyme sensors including environmentally sensitive or fluorescent labels and uses thereof

(75) Inventors: David S. Lawrence, Chapel Hill, NC (US); Qunzhao Wang, Chapel Hill, NC (US)

Correspondence Address: QUINE INTELLECTUAL PROPERTY LAW GROUP, P.C., P O BOX 458, ALAMEDA, CA, 94501, US

(73) Assignee: The Albert Einstein College of Medicine of Yeshiva University

(21) Appl. No.: 12/074,227

(22) Filed: Feb. 28, 2008

Related U.S. Application Data
(63) Continuation of application No. 11/366,221, filed on Mar. 1, 2006, now abandoned.
(60) Provisional application No. 60/658,317, filed on Mar. 2, 2005.
(60) Provisional application No. 60/728,351, filed on Oct. 18, 2005.

(51) Int. Cl. C12Q 1/48

(52) U.S. Cl. 435/15

(57) ABSTRACT

Sensors for detecting enzyme activity are provided. The sensors include substrate modules having environmentally sensitive labels and detection modules whose binding to the substrate modules results in changes in signals from the environmentally sensitive labels or polypeptides or polypeptide substrates including environmentally sensitive or fluorescent labels. Compositions including substrate modules, polypeptides, or polypeptide substrates and nucleic acids encoding enzymes and/or detection modules are also described. Methods of assaying enzyme activity using sensors including environmentally sensitive or fluorescent labels are provided, as are related methods for screening for modulators of enzyme activity.

### CROSS-REFERENCE TO RELATED APPLICATIONS

This application is a non-provisional utility patent application claiming priority to and benefit of the following prior provisional patent applications: U.S. Ser. No. 60/658,317, filed Mar. 2, 2005, entitled “ENZYME SENSORS INCLUDING ENVIRONMENTALLY SENSITIVE LABELS AND USES THEREOF” by David S. Lawrence et al., and U.S. Ser. No. 60/728,351, filed Oct. 18, 2005, entitled “ENZYME SENSORS INCLUDING ENVIRONMENTALLY SENSITIVE OR FLUORESCENT LABELS AND USES THEREOF” by David S. Lawrence, each of which is incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with government support under Grant No. CA79954 from the National Institutes of Health. The government may have certain rights to this invention.

### FIELD OF THE INVENTION

The invention relates to sensors for detecting enzyme activity and uses thereof. The sensors include substrate modules having environmentally sensitive labels and detection modules whose binding to the substrate modules results in changes in signals from the environmentally sensitive labels, or polypeptide substrates having environmentally sensitive or fluorescent labels whose signals change upon phosphorylation or dephosphorylation of the substrates.

### BACKGROUND OF THE INVENTION

Detection of enzyme activity is a necessary step in a wide variety of clinical and basic research applications. For example, in one approach to identifying lead compounds in drug discovery programs, a large number of compounds are screened for activity as inhibitors or activators of a particular enzyme's activity. As just one example, since abnormal protein phosphorylation has been implicated in a number of diseases and pathological conditions including arthritis, cancer, diabetes, and heart disease, screening for compounds capable of modulating the activity of various protein kinases or protein phosphatases can produce lead compounds for evaluation in treatment of these conditions (see, e.g., Ross et al. (2002) “A non-radioactive method for the assay of many serine/threonine-specific protein kinases” Biochem. J. 366:977-998 and references therein).

Simple and reproducible methods for qualitative and/or quantitative detection of enzyme activity are thus desirable, for drug discovery and a wide variety of other applications. Among other benefits, the present invention provides sensors for detecting enzyme activity, as well as related methods for detection of enzyme activity and for screening for compounds affecting enzyme activity.

### SUMMARY OF THE INVENTION

The present invention relates to enzyme sensors including environmentally sensitive and/or fluorescent labels. Compositions including and methods using such sensors or components thereof are described.

A first general class of embodiments provides a composition including an enzyme and a sensor for detecting an activity of the enzyme. The sensor comprises a substrate module and a detection module. The substrate module includes a substrate for the enzyme, wherein the substrate is in a first state on which the enzyme can act, thereby converting the substrate to a second state, and an environmentally sensitive label. The detection module binds to the substrate module when the substrate is in the first state or when the substrate is in the second state. Binding of the detection module to the substrate module results in a change in signal from the label.

Typically, the substrate module comprises a first molecule and the detection module comprises a second molecule. For example, the substrate module can comprise a first polypeptide and the detection module a second polypeptide or an aptamer. The substrate module optionally comprises a polypeptide comprising a (L)-2,3-diaminopropionic acid (Dap), (L)-2,4-diaminobutyric acid (Dab), ornithine, lysine, cysteine, or homocysteine residue to which the environmentally sensitive label is attached.

In one preferred class of embodiments, the enzyme is a protein kinase. In this class of embodiments, the substrate in the first state is unphosphorylated, and the substrate in the second state is phosphorylated. In some embodiments, the detection module binds to the substrate module when the substrate is in the second state (i.e., the detection module binds to the phosphorylated substrate).

In one class of embodiments, the protein kinase is a tyrosine protein kinase. In this class of embodiments, the substrate module optionally comprises a first polypeptide and the detection module a second polypeptide including an SH2 domain, a PTB domain, or an antibody. In another class of embodiments, the protein kinase is a serine/threonine protein kinase. In this class of embodiments, the substrate module optionally comprises a first polypeptide and the detection module a second polypeptide including a 14-3-3 domain or an antibody.

In another preferred class of embodiments, the enzyme is a protein phosphatase. In this class of embodiments, the substrate in the first state is phosphorylated, and the substrate in the second state is unphosphorylated. In some embodiments, the detection module binds to the substrate module when the substrate is in the first state (i.e., the detection module binds to the phosphorylated substrate).

In one exemplary class of embodiments, the substrate module includes a polypeptide having amino acid sequence X⁻⁴ X⁻³ X⁻² X⁻¹ Y⁰ X⁺¹ X⁺² X⁺³ X⁺⁴ X⁺⁵; where X⁻⁴, X⁻³, and X⁻² are independently selected from the group consisting of: D, E, and an amino acid residue comprising the environmentally sensitive label; X⁻¹ and X⁺³ are independently selected from the group consisting of: A, V, I, L, M, F, Y, W, and an amino acid residue comprising the environmentally sensitive label; X⁺¹, X⁺², X⁺⁴, and X⁺⁵ are independently selected from the group consisting of: an amino acid residue and an amino acid residue comprising the environmentally sensitive label; and at least one of X⁻⁴, X⁻³, X⁻², X⁻¹, X⁺¹, X⁺², X⁺³, X⁺⁴, and X⁺⁵ is an amino acid residue comprising the environmentally sensitive label. For example, one of X⁺¹, X⁺², X⁺³, and X⁺⁴ can be an amino acid residue comprising the environmentally sensitive label. In one class of embodiments, the substrate module includes a polypeptide comprising an amino acid sequence selected from the group consisting of: EEEIYX⁺¹EIEA (SEQ ID NO:1) where X⁺¹ is an amino acid residue comprising the environmentally sensitive label, EEEIYGX⁺²IEA (SEQ ID NO:2) where X⁺² is an amino acid residue comprising the environmentally sensitive label, EEEIYGEX⁺³EA (SEQ ID NO:3) where X⁺³ is an amino acid residue comprising the environmentally sensitive label, and EEEIYGEIX⁺⁴A (SEQ ID NO:4) where X⁺⁴ is an amino acid residue comprising the environmentally sensitive label (e.g., a Dap, Dab, ornithine, lysine, cysteine, or homocysteine residue). For example, the substrate module can include a polypeptide comprising the amino acid sequence EEEIYGEIX⁺⁴A, where X⁺⁴ comprises a dapoxyl group attached to a Dab residue (SEQ ID NO:7); wherein the polypeptide substrate comprises a polypeptide comprising the amino acid sequence EEEIYGEX⁺³EA, where X⁺³ comprises a dapoxyl group attached to a Dab residue (SEQ ID NO:10); or wherein the polypeptide substrate comprises a polypeptide comprising the amino acid sequence EEEIYGEX⁺³EA, where X⁺³ comprises a dapoxyl group attached to a Dap residue (SEQ ID NO:11). The enzyme is optionally a tyrosine protein kinase (e.g., Src kinase) or a protein phosphatase (e.g., a tyrosine-specific protein phosphatase).

In one class of embodiments, the label is a fluorescent label. The change in signal from the label can be a change in fluorescence emission intensity, e.g., a change of greater than ±25%, greater than ±50%, greater than ±75%, greater than ±90%, greater than ±95%, greater than ±98%, greater than +100%, greater than +200%, greater than +300%, greater than +400%, greater than +500%, greater than +600%, or greater than +700% in fluorescence emission intensity. The label optionally comprises a label selected from the group consisting of: NBD, Cascade Yellow, dapoxyl, pyrene, bimane, 7-diethylaminocoumarin-3-carboxylic acid, Marina Blue™, Pacific Blue™, Cascade Blue™, 2-anthracenesulfonyl, dansyl, PyMPO, and 3,4,9,10-perylene-tetracarboxylic acid.

The composition optionally includes a cell lysate or a cell, e.g., a cell comprising the sensor, a cell comprising the enzyme, or a cell comprising the enzyme and the sensor. The composition optionally includes a modulator or potential modulator of the activity of the enzyme.

The substrate module is optionally associated with a cellular delivery module that can mediate introduction of the substrate module into a cell, e.g., a polypeptide, a PEP-1 peptide, an amphipathic peptide, a cationic peptide, or a protein transduction domain. Similarly, the composition can include cyclodextran associated with the substrate module. The detection module is optionally associated with a cellular delivery module that can mediate introduction of the detection module into the cell. Alternatively, the detection module can be endogenous to the cell.

In one class of embodiments, the sensor comprises one or more caging groups associated with the substrate module. The caging groups inhibit the enzyme from acting upon the substrate, e.g., by at least about 75%, at least about 90%, at least about 95%, or at least about 98%, as compared to the substrate in the absence of the one or more caging groups. Preferably, the one or more caging groups prevent the enzyme from acting upon the substrate. Typically, removal of, or an induced conformational change in, the one or more caging groups permits the enzyme to act upon the substrate. The one or more caging groups associated with the substrate module can be covalently or non-covalently attached to the substrate module. In a preferred aspect, the one or more caging groups are photoactivatable (e.g., photolabile).

Another general class of embodiments provides a composition that includes a polypeptide (typically, a polypeptide substrate) comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label; X⁻¹ and X⁺³ are independently selected from the group consisting of: A, V, I, L, M, F, Y, W, and an amino acid residue comprising the environmentally sensitive or fluorescent label; and X⁺¹, X⁺², X⁺⁴, and X⁺⁵ are independently selected from the group consisting of: an amino acid residue and an amino acid residue comprising the environmentally sensitive or fluorescent label. At least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label.

In one class of embodiments, one of X⁺¹, X⁺², X⁺³, and X⁺⁴ is an amino acid residue comprising the environmentally sensitive or fluorescent label. For example, the polypeptide can comprise an amino acid sequence selected from the group consisting of: EEEIYX⁺¹EIEA (SEQ ID NO:1) where X⁺¹ is an amino acid residue comprising the environmentally sensitive or fluorescent label, EEEIYGX⁺²IEA (SEQ ID NO:2) where X⁺² is an amino acid residue comprising the environmentally sensitive or fluorescent label, EEEIYGEX⁺³EA (SEQ ID NO:3) where X⁺³ is an amino acid residue comprising the environmentally sensitive or fluorescent label, and EEEIYGEIX⁺⁴A (SEQ ID NO:4) where X⁺⁴ is an amino acid residue comprising the environmentally sensitive or fluorescent label. X⁺¹, X⁺², X⁺³, or X⁺⁴ optionally comprises a Dap, Dab, ornithine, lysine, cysteine, or homocysteine residue, or essentially any other residue to which the label can be attached. Thus, for example, the polypeptide optionally comprises the amino acid sequence EEEIYGEIX⁺⁴A, where X⁺⁴ comprises a dapoxyl group attached to a Dab residue (SEQ ID NO:7), the amino acid sequence EEEIYGEX⁺³EA, where X⁺³ comprises a dapoxyl group attached to a Dab residue (SEQ ID NO:10), or the amino acid sequence EEEIYGEX⁺³EA, where X⁺³ comprises a dapoxyl group attached to a Dap residue (SEQ ID NO:11).

In one class of embodiments, one of X⁻² and X⁺³ is an amino acid residue comprising the environmentally sensitive or fluorescent label. For example, the polypeptide optionally comprises an amino acid sequence selected from the group consisting of: EEX⁻²IYGEIEA (SEQ ID NO:9), where X⁻² is an amino acid residue comprising the environmentally sensitive or fluorescent label, and EEEIYGEX⁺³EA (SEQ ID NO:3), where X⁺³ is an amino acid residue comprising the environmentally sensitive or fluorescent label. X⁻² or X⁺³ optionally comprises a Dap, Dab, ornithine, lysine, cysteine, or homocysteine residue. Thus, for example, the polypeptide can comprise the amino acid sequence EEX⁻²IYGEIEA, where X⁻² comprises pyrene attached to a Dab residue (SEQ ID NO:12), the amino acid sequence EEEIYGEX⁺³EA, where X⁺³ comprises pyrene attached to a Dab residue (SEQ ID NO:13), the amino acid sequence EEEIYGEX⁺³EA, where X⁺³ comprises pyrene attached to a Dap residue (SEQ ID NO:14), the amino acid sequence EEX⁻²IYGEIEA, where X⁻² comprises Cascade Yellow attached to a Dab residue (SEQ ID NO:15), the amino acid sequence EEX⁻²IYGEIEA, where X⁻² comprises 2,7-difluorofluorescein (Oregon Green™ 488-X) attached to a Dab residue (SEQ ID NO:17), the amino acid sequence EEEIYGEX⁺³EA, where X⁺³ comprises 2,7-difluorofluorescein (Oregon Green™ 488-X) attached to a Dap residue (SEQ ID NO:18), the amino acid sequence EEX²IYGEIEA, where X⁻² comprises Cascade Blue™ attached to a Dab residue (SEQ ID NO:19), or the amino acid sequence EEEIYGEX⁺³EA, where X⁺³ comprises Cascade Blue™ attached to a Dap residue (SEQ ID NO:20).

In one class of embodiments, the label is a fluorescent label. The label optionally comprises a label selected from the group consisting of: NBD, Cascade Yellow, dapoxyl, pyrene, 2,7-difluorofluorescein (Oregon Green™ 488-X), 7-diethylaminocoumarin-3-carboxylic acid, 5-carboxyfluorescein, Texas Red™-X, Marina Blue™, Pacific Blue™, Cascade Blue™, bimane, 2-anthracenesulfonyl, dansyl, Alexa Fluor 430, PyMPO, 5-carboxytetramethylrhodamine (5-TAMRA), 6-carboxytetramethylrhodamine (6-TAMRA), BODIPY FL, and 3,4,9,10-perylene-tetracarboxylic acid, and derivatives thereof.

In one class of embodiments, the composition further comprises a tyrosine protein kinase, e.g., a kinase selected from the group consisting of Src, SrcN1, SrcN2, FynT, Fgr, Lck, Yes, LynA, LynB, Hck, Abl, Csk, Fes/Fps, FGFR, TrkA, and Flt3, or another tyrosine kinase for which the polypeptide is, or is suspected to be, a substrate. In another class of embodiments, the composition further comprises a protein phosphatase, typically, a tyrosine-specific protein phosphatase for which the polypeptide is, or is suspected to be, a substrate.

The tyrosine at the phosphorylation site, Y⁰, optionally comprises a free hydroxyl group (i.e., is unphosphorylated), or is optionally a phosphorylated tyrosine residue.

Preferably, phosphorylation (or, correspondingly, dephosphorylation) of Y⁰ results in a change in signal from the label. The change in signal from the label can be a change in fluorescence emission intensity, e.g., a change of greater than ±25%, greater than ±50%, greater than ±75%, greater than ±90%, greater than ±95%, greater than ±98%, greater than +100%, greater than +200%, greater than +300%, greater than +400%, greater than +500%, greater than +600%, or greater than +700% in fluorescence emission intensity.

In one class of embodiments, the change in signal depends on the presence of a detection module. Thus, in this class of embodiments, the composition optionally also includes a second polypeptide comprising an SH2 domain, a PTB domain, or an antibody. Binding of the second polypeptide to the phosphorylated substrate leads to the change in signal. In a preferred class of embodiments, however, no detection module is required for the change in signal to result from phosphorylation (or dephosphorylation) of Y⁰. In this class of embodiments, no detection module, second polypeptide, or the like need be present in the composition. In this class of embodiments, for example, the change in signal can result from a phosphorylation-induced change in the local environment of an environmentally sensitive label, from disruption of an interaction between a fluorescent or environmentally sensitive label and Y⁰ upon phosphorylation of Y⁰, and/or the like.

Essentially all of the features noted above apply to this class of embodiments as well, as relevant; for example, with respect to type of kinase or phosphatase, use of cellular delivery modules, inclusion of a nucleic acid encoding a kinase or phosphatase whose activity is to be detected, inclusion of a modulator or potential modulator of the activity of the enzyme, and/or the like.

Thus, for example, the sensors can be used in biochemical assays of enzyme activity, to detect enzyme activity inside cells and/or organisms, or the like. Thus, the composition optionally includes a cell lysate or a cell, e.g., a cell comprising the sensor, a cell comprising the enzyme, or a cell comprising the enzyme and the sensor.

As another example, the sensor is optionally caged. Thus, in one class of embodiments, the composition comprises one or more caging groups associated with the polypeptide. The caging groups inhibit an enzyme from acting upon the polypeptide, e.g., by at least about 75%, at least about 90%, at least about 95%, or at least about 98%, as compared to the polypeptide in the absence of the one or more caging groups. Preferably, the one or more caging groups prevent the enzyme from acting upon the polypeptide. Typically, removal of, or an induced conformational change in, the one or more caging groups permits the enzyme to act upon the polypeptide. The one or more caging groups associated with the polypeptide can be covalently or non-covalently attached to the polypeptide. For example, a single caging group can be covalently attached to the Y⁰ side chain. In a preferred aspect, the one or more caging groups are photoactivatable (e.g., photolabile).

Yet another general class of embodiments provides a composition that includes a polypeptide (typically, a polypeptide substrate) comprising an environmentally sensitive or fluorescent label. The polypeptide comprises a tyrosine residue, and when the tyrosine is unphosphorylated, it engages in an interaction with the label. This interaction is at least partially disrupted when the tyrosine is phosphorylated, whereby a signal from the label changes upon phosphorylation or dephosphorylation of the tyrosine.

In one class of embodiments, the environmentally sensitive or fluorescent label comprises an aromatic ring. When the tyrosine is unphosphorylated, it engages in an interaction with the aromatic ring of the label, and the interaction is at least partially disrupted when the tyrosine is phosphorylated. For example, when the tyrosine is unphosphorylated, it can engage in a π-π stacking interaction or an edge-face interaction with the aromatic ring of the label.

In one class of embodiments, the composition further comprises a tyrosine protein kinase, typically, a kinase for which the polypeptide is, or is suspected to be, a substrate. In another class of embodiments, the composition further comprises a protein phosphatase, typically, a tyrosine-specific protein phosphatase for which the polypeptide is, or is suspected to be, a substrate.

In one exemplary class of embodiments, the polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label; X⁻¹ and X⁺³ are independently selected from the group consisting of: A, V, I, L, M, F, Y, W, and an amino acid residue comprising the environmentally sensitive or fluorescent label; and X⁺¹, X⁺², X⁺⁴, and X⁺⁵ are independently selected from the group consisting of: an amino acid residue and an amino acid residue comprising the environmentally sensitive or fluorescent label. At least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label.

In one class of embodiments, one of X⁻² and X⁺³ is an amino acid residue comprising the environmentally sensitive or fluorescent label. For example, the polypeptide optionally comprises an amino acid sequence selected from the group consisting of: EEX⁻²IYGEIEA (SEQ ID NO:9), where X⁻² is an amino acid residue comprising the environmentally sensitive or fluorescent label, and EEEIYGEX⁺³EA (SEQ ID NO:3), where X⁺³ is an amino acid residue comprising the environmentally sensitive or fluorescent label. X⁻² or X⁺³ optionally comprises a Dap, Dab, ornithine, lysine, cysteine, or homocysteine residue, or essentially any other residue to which the label can be attached. Thus, for example, the polypeptide can comprise the amino acid sequence EEX⁻²IYGEIEA, where X⁻² comprises pyrene attached to a Dab residue (SEQ ID NO:12), the amino acid sequence EEEIYGEX⁺³EA, where X⁺³ comprises pyrene attached to a Dab residue (SEQ ID NO:13), the amino acid sequence EEEIYGEX⁺³EA, where X⁺³ comprises pyrene attached to a Dap residue (SEQ ID NO:14), the amino acid sequence EEX⁻²IYGEIEA, where X⁻² comprises Cascade Yellow attached to a Dab residue (SEQ ID NO:15), the amino acid sequence EEX⁻²IYGEIEA, where X⁻² comprises 2,7-difluorofluorescein (Oregon Green™ 488-X) attached to a Dab residue (SEQ ID NO:17), the amino acid sequence EEEIYGEX⁺³EA, where X⁺³ comprises 2,7-difluorofluorescein (Oregon Green™ 488-X) attached to a Dap residue (SEQ ID NO:18), the amino acid sequence EEX⁻²IYGEIEA, where X⁻² comprises Cascade Blue™ attached to a Dab residue (SEQ ID NO:19), or the amino acid sequence EEEIYGEX⁺³EA, where X⁺³ comprises Cascade Blue™ attached to a Dap residue (SEQ ID NO:20).

Essentially all of the features noted above apply to this class of embodiments as well, as relevant; for example, with respect to type of label, signal change from the label, type of kinase or phosphatase, inclusion of a second sensor in the composition, use of cellular delivery modules, inclusion of a nucleic acid encoding a kinase or phosphatase whose activity is to be detected, inclusion of a modulator or potential modulator of the activity of the enzyme, caging of the polypeptide, inclusion of a cell or cell lysate, and/or the like.

Yet another general class of embodiments provides a composition that includes a polypeptide substrate for a protein tyrosine kinase or a tyrosine-specific protein phosphatase. The polypeptide substrate comprises an environmentally sensitive or fluorescent label, which is located at amino acid position −2 or +3 with respect to the phosphorylation site (the tyrosine that is phosphorylated by the kinase or dephosphorylated by the phosphatase) within the polypeptide substrate.

In a preferred class of embodiments, phosphorylation or dephosphorylation of the substrate at the phosphorylation site results in a change in signal from the label. In one class of embodiments, the label is a fluorescent label such as those described herein.

In one exemplary class of embodiments, the polypeptide substrate comprises a polypeptide having amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label; X⁻¹ and X⁺³ are independently selected from the group consisting of: A, V, I, L, M, F, Y, W, and an amino acid residue comprising the environmentally sensitive or fluorescent label; and X⁺¹, X⁺², X⁺⁴, and X⁺⁵ are independently selected from the group consisting of: an amino acid residue and an amino acid residue comprising the environmentally sensitive or fluorescent label. At least one of X⁻² and X⁺³ is an amino acid residue comprising the environmentally sensitive or fluorescent label. For example, the polypeptide optionally comprises an amino acid sequence selected from the group consisting of: EEX⁻²IYGEIEA (SEQ ID NO:9), where X⁻² is an amino acid residue comprising the environmentally sensitive or fluorescent label, and EEEIYGEX⁺³EA (SEQ ID NO:3), where X⁺³ is an amino acid residue comprising the environmentally sensitive or fluorescent label. X⁻² or X⁺³ optionally comprises a Dap, Dab, ornithine, lysine, cysteine, or homocysteine residue, or essentially any other residue to which the label can be attached. Thus, for example, the polypeptide can comprise the amino acid sequence EEX⁻²IYGEIEA, where X⁻² comprises pyrene attached to a Dab residue (SEQ ID NO:12), the amino acid sequence EEEIYGEX⁺³EA, where X⁺³ comprises pyrene attached to a Dab residue (SEQ ID NO:13), the amino acid sequence EEEIYGEX⁺³EA, where X⁺³ comprises pyrene attached to a Dap residue (SEQ ID NO:14) the amino acid sequence EEX⁻²IYGEIEA, where X⁻² comprises Cascade Yellow attached to a Dab residue (SEQ ID NO:15), the amino acid sequence EEX⁻²IYGEIEA, where X⁻² comprises 2,7-difluorofluorescein (Oregon Green™ 488-X) attached to a Dab residue (SEQ ID NO:17), the amino acid sequence EEEIYGEX⁺³EA, where X⁺³ comprises 2,7-difluorofluorescein (Oregon Green™ 488-X) attached to a Dap residue (SEQ ID NO:18), the amino acid sequence EEX⁻²IYGEIEA, where X⁻² comprises Cascade Blue™ attached to a Dab residue (SEQ ID NO:19), or the amino acid sequence EEEIYGEX⁺³EA, where X⁺³ comprises Cascade Blue™ attached to a Dap residue (SEQ ID NO:20).

Essentially all of the features noted above apply to this class of embodiments as well, as relevant; for example, with respect to inclusion and type of kinase or phosphatase, type of label, signal change from the label, use of cellular delivery modules, inclusion of a nucleic acid encoding a kinase or phosphatase whose activity is to be detected, inclusion of a modulator or potential modulator of the activity of the enzyme, caging of the polypeptide, inclusion of a cell or cell lysate, and/or the like.

Another general class of embodiments provides methods of assaying an activity of an enzyme. In the methods, the enzyme is contacted with a sensor. The sensor includes 1) a substrate module comprising a substrate for the enzyme, wherein the substrate is in a first state on which the enzyme can act, thereby converting the substrate to a second state, and an environmentally sensitive label, and 2) a detection module, which detection module binds to the substrate module when the substrate is in the first state or the second state. Binding of the detection module to the substrate module results in a change in signal from the label. The change in signal from the label is detected, and the activity of the enzyme is assayed by correlating the change in signal from the label to the activity of the enzyme.

The methods can be used, e.g., for in vitro biochemical assays of enzyme activity using purified or partially purified enzyme, a cell lysate, or the like, or they can be used to detect enzyme activity inside cells and/or organisms. Thus, in one class of embodiments, contacting the enzyme and the sensor comprises introducing the substrate module into a cell. Similarly, in some embodiments, contacting the enzyme and the sensor comprises introducing the detection module into the cell. In other embodiments, the methods include introducing a vector encoding the detection module into the cell, whereby the detection module is expressed in the cell. Similarly, in one class of embodiments, a vector encoding the enzyme is introduced into the cell, whereby the enzyme is expressed in the cell.

In one class of embodiments, the sensor comprises one or more caging groups associated with the substrate module, which caging groups inhibit (e.g., prevent) the enzyme from acting upon the substrate. The methods include uncaging the substrate module, e.g., by exposing the substrate module to light of a first wavelength, thereby freeing the substrate module from inhibition by the one or more caging groups. Typically, the one or more caging groups prevent the enzyme from acting upon the substrate, and removal of or an induced conformational change in the one or more caging groups permits the enzyme to act upon the substrate.

In a preferred aspect, the environmentally sensitive label is a fluorescent label. The change in signal from the label can thus be a change in fluorescence emission intensity, e.g., a change of greater than ±25%, greater than ±50%, greater than ±75%, greater than ±90%, greater than ±95%, greater than ±98%, greater than +100%, greater than +200%, greater than +300%, greater than +400%, greater than +500%, greater than +600%, or greater than +700% in fluorescence emission intensity.

In one class of embodiments the methods include contacting the enzyme with a test compound, assaying the activity of the enzyme in the presence of the test compound, and comparing the activity of the enzyme in the presence of the test compound with the activity of the enzyme in the absence of the test compound.

Essentially all of the features noted for the compositions above apply to these methods as well, as relevant: for example, with respect to type of enzyme, exemplary substrate and detection modules, fluorescent labels, type of caging groups, use of cellular delivery modules, and/or the like.

Another general class of embodiments also provides methods of assaying an activity of an enzyme (e.g., a tyrosine kinase or tyrosine-specific phosphatase). In the methods, the enzyme is contacted with a sensor, whereby the enzyme optionally phosphorylates or dephosphorylates the sensor. The sensor includes an environmentally sensitive or fluorescent label whose signal changes upon phosphorylation or dephosphorylation of the sensor. The change in signal from the label is detected and correlated to the activity of the enzyme, whereby the activity of the enzyme is assayed.

In one class of embodiments, the sensor includes a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label, X⁻¹ and X⁺³ are independently selected from the group consisting of: A, V, I, L, M, F, Y, W, and an amino acid residue comprising the environmentally sensitive or fluorescent label, X⁻¹, X⁺², X⁺⁴, and X⁺⁵ are independently selected from the group consisting of: an amino acid residue and an amino acid residue comprising the environmentally sensitive or fluorescent label, and at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label. Phosphorylation or dephosphorylation of Y⁰ results in a change in signal from the label.

In another class of embodiments, the sensor includes a polypeptide comprising an environmentally sensitive or fluorescent label, which polypeptide comprises a tyrosine residue. When the tyrosine is unphosphorylated, it engages in an interaction with the label, and this interaction is at least partially disrupted when the tyrosine is phosphorylated, whereby a signal from the label changes upon phosphorylation or dephosphorylation of the tyrosine.

In yet another class of embodiments, the sensor includes a polypeptide substrate for a protein tyrosine kinase, which polypeptide substrate comprises an environmentally sensitive or fluorescent label. The environmentally sensitive or fluorescent label is located at amino acid position −2 or +3 with respect to the phosphorylation site within the polypeptide substrate, and phosphorylation or dephosphorylation of the substrate at the phosphorylation site results in a change in signal from the label.

The methods can be used, e.g., for in vitro biochemical assays of enzyme activity using purified or partially purified enzyme, a cell lysate, or the like, or they can be used to detect enzyme activity inside cells and/or organisms. Thus, in one class of embodiments, contacting the enzyme and the sensor comprises introducing the sensor into a cell, e.g., a cell including or potentially including the enzyme.

In a preferred aspect, the label is a fluorescent label. The change in signal from the label can be a change in fluorescence emission intensity, e.g., a change of greater than ±25%, greater than ±50%, greater than ±75%, greater than ±90%, greater than ±95%, greater than ±98%, greater than +100%, greater than +200, greater than +300%, greater than +400%, greater than +500%, greater than +600%, or greater than +700% in fluorescence emission intensity.

As noted previously, caging the sensor can permit initiation of the activity assay to be precisely controlled, temporally and/or spatially. Thus, in one class of embodiments, the sensor comprises one or more caging groups associated with the polypeptide or polypeptide substrate, which caging groups inhibit (e.g., prevent) the enzyme from acting upon the polypeptide or polypeptide substrate. The methods include uncaging the polypeptide or polypeptide substrate, e.g., by exposing the caged sensor to uncaging energy, thereby freeing the polypeptide or polypeptide substrate from inhibition by the one or more caging groups. Typically, the one or more caging groups prevent the enzyme from acting upon the polypeptide or polypeptide substrate, and removal of or an induced conformational change in the one or more caging groups permits the enzyme to act upon the polypeptide or polypeptide substrate. The caged polypeptide or polypeptide substrate can be uncaged, for example, by exposing the caged sensor to light of a first wavelength (for photoactivatable or photolabile caging groups), sonicating the caged sensor, or otherwise supplying uncaging energy appropriate for the specific caging groups utilized.

In one aspect, the methods can be used to screen for compounds that affect activity of the enzyme. Thus, in one class of embodiments, the methods include contacting the enzyme with a test compound, assaying the activity of the enzyme in the presence of the test compound, and comparing the activity of the enzyme in the presence of the test compound with the activity of the enzyme in the absence of the test compound.

Essentially all of the features noted for the compositions and methods above apply to these methods as well, as relevant: for example, with respect to type of enzyme, exemplary sensors, fluorescent labels, type of caging groups, use of cellular delivery modules, and/or the like.

Yet another general class of embodiments provides methods of determining whether a test compound affects an activity of an enzyme. In the methods, a cell comprising the enzyme is provided, and a sensor is introduced into the cell.

In one class of embodiments, the sensor includes a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label, X⁻¹ and X⁺³ are independently selected from the group consisting of: A, V, I, L, M, F, Y, W, and an amino acid residue comprising the environmentally sensitive or fluorescent label, X⁺¹, X⁺², X⁺⁴, and X⁺⁵ are independently selected from the group consisting of: an amino acid residue and an amino acid residue comprising the environmentally sensitive or fluorescent label, and at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label. Phosphorylation or dephosphorylation of Y⁰ results in a change in signal from the label.

In another class of embodiments, the sensor includes a polypeptide comprising an environmentally sensitive or fluorescent label, which polypeptide comprises a tyrosine residue. When the tyrosine is unphosphorylated, it engages in an interaction with the label, and this interaction is at least partially disrupted when the tyrosine is phosphorylated, whereby a signal from the label changes upon phosphorylation or dephosphorylation of the tyrosine.

In yet another class of embodiments, the sensor includes a polypeptide substrate for a protein tyrosine kinase, which polypeptide substrate comprises an environmentally sensitive or fluorescent label. The environmentally sensitive or fluorescent label is located at amino acid position −2 or +3 with respect to the phosphorylation site within the polypeptide substrate, and phosphorylation or dephosphorylation of the substrate at the phosphorylation site results in a change in signal from the label.

In yet another class of embodiments, the sensor includes 1) a substrate module comprising a substrate for the enzyme, wherein the substrate is in a first state on which the enzyme can act, thereby converting the substrate to a second state, and an environmentally sensitive label, and 2) a detection module, which detection module binds to the substrate module when the substrate is in the first state or the second state, wherein binding of the detection module to the substrate module results in a change in signal from the label.

Regardless of which type of sensor is employed, the cell is contacted with the test compound, and the change in signal from the label is detected. The change provides an indication of the activity of the enzyme in the presence of the test compound. Typically, the activity of the enzyme in the presence of the test compound is compared to an activity of the enzyme in the absence of the test compound, to determine whether the test compound increases, decreases, or does not substantially affect the enzyme's activity.

In one class of embodiments, providing the cell comprising the enzyme comprises introducing a vector encoding the enzyme into the cell, whereby the enzyme is expressed in the cell. In embodiments in which the sensor includes a substrate module and a detection module, introducing the sensor into the cell optionally comprises introducing the substrate module and the detection module into the cell. In another exemplary class of embodiments, introducing the sensor into the cell comprises introducing the substrate module and a vector encoding the detection module into the cell, whereby the detection module is expressed in the cell.

Essentially all of the features noted for the compositions and methods above apply to these methods as well, as relevant: for example, with respect to type of enzyme (e.g., kinase or phosphatase), exemplary sensors, fluorescent labels, use of caging groups, use of cellular delivery modules, and/or the like.

### BRIEF DESCRIPTION OF THE DRAWINGS

FIG. 1 schematically illustrates phosphorylation of fluorophore-labeled peptide substrates, in which the fluorophore is appended directly to the phosphorylatable residue (1→2) or in which a divalent metal ion interacts with the fluorophore and the phosphorylated residue (3→4).

FIG. 2 presents exemplary fluorophores: a dapoxyl derivative (5), NBD (6), and a Cascade Yellow derivative (7).

FIG. 3 Panel A schematically illustrates phosphorylation of an exemplary peptide substrate (SEQ ID NO:4) labeled with an environmentally sensitive fluorophore by Src kinase and then binding of the phosphorylated substrate by an SH2 domain, leading to increased fluorescence from the environmentally sensitive fluorophore. Panel B schematically illustrates phosphorylation of a kinase peptide substrate labeled with an environmentally sensitive fluorophore by Src kinase and then binding of the phosphorylated substrate by an Lck SH2 domain, leading to increased fluorescence from the environmentally sensitive fluorophore.

FIG. 4 schematically illustrates the structures of a Dap residue (11), a Dab residue (12), an exemplary peptide substrate indicating the location of residue positions P+1−P+4 (SEQ ID NO:5), an exemplary NBD-labeled substrate (13, SEQ ID NO:6), and an exemplary dapoxyl-labeled substrate (14, SEQ ID NO:7).

FIG. 5 presents a graph illustrating fluorescence change from exemplary labeled and phosphorylated substrate 13 as a function of the concentration of the Lck SH2 domain.

FIG. 6 presents a graph illustrating fluorescence from exemplary labeled and phosphorylated substrate 13 in the presence of the Lck SH2 domain ligand YEEIE (SEQ ID NO:8) or in the presence of phosphatase PTP1B added either with ATP or following SRC-catalyzed phosphorylation of the substrate.

FIG. 7 schematically illustrates the structures of an exemplary peptide substrate indicating the location of residue positions Y−2 and Y+1−Y+4 (SEQ ID NO:5), a Dap residue (21), a Dab residue (22), unphosphorylated (23) and phosphorylated (24) versions of an exemplary pyrene-labeled substrate (SEQ ID NO:14), and another exemplary pyrene-labeled substrate (25, SEQ ID NO:12).

FIG. 8 presents a graph of fluorescence change as a function of time for the Src kinase-catalyzed phosphorylation of peptide 23 (20 μM).

FIG. 9 presents a graph illustrating phosphorylation-induced fold fluorescence change as a function of Dap-pyrene (black) and Dab-pyrene (white) position. The structure of the exemplary peptide substrate indicating the location of residue positions Y−2 and Y+1−Y+4 (SEQ ID NO:5) is also shown, as are the structures of Dap and Dab.

FIG. 10 Panel A presents a 2D NOESY spectrum (450 ms mixing time) of the unphosphorylated peptide 23 showing NOEs between the pyrene aromatic protons (for designations and assignments, see Panel C and Tables 5-7) and the tyrosine aromatic protons. Panel B presents a 2D NOESY spectrum (450 ms mixing time) of the phosphorylated peptide 24 showing NOEs between the pyrene and tyrosine aromatic protons. Panel C indicates pyrene proton designations for Panels A and B.

FIG. 11 presents a schematic model of the interaction between the pyrene and phenol substituents based on the NOE and chemical shift data. The double-headed arrow indicates that NOEs between the benzylic protons are observed as well.

FIG. 12 Panel A presents a graph illustrating Brk-catalyzed phosphorylation of peptide 23. Curve a represents fluorescence emission (Flem) versus time for the Brk-catalyzed phosphorylation of peptide 23 initiated by addition of ATP. The biphasic reaction progress curve is highlighted by an initial lag period. Curve b represents Flem versus time for the Brk-catalyzed phosphorylation of peptide 23 initiated by addition of pyrene-peptide 23. Brk and ATP were pre-incubated for 120 min prior to addition of 23. Panel B presents a graph illustrating initial phosphorylation rate versus pre-incubation time of Brk and ATP.

FIG. 13 schematically illustrates exemplary Cascade Yellow-labeled substrates (26, SEQ ID NO:15 and 27, SEQ ID NO:16), an exemplary Oregon Green™-labeled substrate (28, SEQ ID NO:17), and an exemplary Cascade Blue™-labeled substrate (29, SEQ ID NO:19).

FIG. 14 presents a graph illustrating phosphorylation-induced fluorescence change as a function of time for the Src-catalyzed phosphorylation of peptide 26 in cell lysate, in the presence and absence of an SH3 domain ligand.

FIG. 15 Panel A schematically illustrates uncaging of exemplary caged sensor 30 to produce active sensor 26. Panel B presents a graph illustrating photoactivation of the caged sensor.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an,” and “the” include plural referents unless the context clearly dictates otherwise. Thus, for example, reference to “a cellular delivery module” includes a plurality of cellular delivery modules, reference to “a cell” includes mixtures of cells, and the like.

The term “about” as used herein indicates the value of a given quantity varies by +/−10% of the value, or optionally +/−5% of the value, or in some embodiments, by +/−1% of the value so described.

An “amino acid sequence” is a polymer of amino acid residues (a protein, polypeptide, etc.) or a character string representing an amino acid polymer, depending on context.

An “aptamer” is a nucleic acid capable of interacting with a ligand. An aptamer can be, e.g., a DNA or RNA, and can be e.g. a chemically synthesized oligonucleotide. The ligand can be any natural or synthetic molecule, including, e.g., the first or second state of a substrate.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into a Fab′ monomer. The Fab′ monomer is essentially a Fab with part of the hinge region (see Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, includes antibodies or fragments either produced by the modification of whole antibodies or synthesized de novo using recombinant DNA methodologies. Antibodies include multiple or single chain antibodies, including single chain Fv (sFv or scFv) antibodies in which a variable heavy and a variable light chain are joined together (directly or through a peptide linker) to form a continuous polypeptide.

A “caging group” is a moiety that can be employed to reversibly block, inhibit, or interfere with the activity (e.g., the biological activity) of a molecule (e.g., a polypeptide, a nucleic acid, a small molecule, a drug, etc.). The caging groups can, e.g., physically trap an active molecule inside a framework formed by the caging groups. Typically, however, one or more caging groups are associated (covalently or noncovalently) with the molecule but do not necessarily surround the molecule in a physical cage. For example, a single caging group covalently attached to an amino acid side chain required for the catalytic activity of an enzyme can block the activity of the enzyme. The enzyme would thus be caged even though not physically surrounded by the caging group. As another example, covalent attachment of a single caging group to an amino acid side chain that is phosphorylated by a kinase in a kinase substrate can block phosphorylation of that substrate by the kinase. Caging groups can be, e.g., relatively small moieties such as carboxyl nitrobenzyl, 2-nitrobenzyl, nitroindoline, hydroxyphenacyl, DMNPE, or the like, or they can be, e.g., large bulky moieties such as a protein or a bead. Caging groups can be removed from a molecule, or their interference with the molecule's activity can be otherwise reversed or reduced, by exposure to an appropriate type of uncaging energy and/or exposure to an uncaging chemical, enzyme, or the like.

A “photoactivatable” or “photoactivated” caging group is a caging group whose blockage of, inhibition of, or interference with the activity of a molecule with which the photoactivatable caging group is associated can be reversed or reduced by exposure to light of an appropriate wavelength. For example, exposure to light can disrupt a network of caging groups physically surrounding the molecule, reverse a noncovalent association with the molecule, trigger a conformational change that renders the molecule active even though still associated with the caging group, or cleave a photolabile covalent attachment to the molecule, etc.

A “photolabile” caging group is one whose covalent attachment to a molecule is reversed (cleaved) by exposure to light of an appropriate wavelength. The photolabile caging group can be, e.g., a relatively small moiety such as carboxyl nitrobenzyl, 2-nitrobenzyl, nitroindoline, hydroxyphenacyl, DMNPE, or the like, or it can be, e.g., a relatively bulky group (e.g. a macromolecule, a protein) covalently attached to the molecule by a photolabile linker (e.g., a polypeptide linker comprising a 2-nitrophenyl glycine residue).

A “cellular delivery module” is a moiety that can mediate introduction into a cell of a molecule with which the module is associated (covalently or noncovalently).

As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term “encode” describes the process of semi-conservative DNA replication, where one strand of a double-stranded DNA molecule is used as a template to encode a newly synthesized complementary sister strand by a DNA-dependent DNA polymerase. In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. In another aspect, a DNA molecule can encode a polypeptide, where it is understood that “encode” as used in that case incorporates both the processes of transcription and translation.

An “enzyme” is a biological macromolecule that has at least one catalytic activity (i.e., that catalyzes at least one chemical reaction). An enzyme is typically a protein, but can be, e.g., RNA. Known protein enzymes have been grouped into six classes (and a number of subclasses and sub-subclasses) under the Enzyme Commission classification scheme (see, e.g. the Nomenclature Committee of the International Union of Biochemistry and Molecular Biology enzyme nomenclature pages, on the world wide web at (www.) chem.qmul.ac.uk/iubmb/enzyme), namely, oxidoreductase, transferase, hydrolase, lyase, ligase, or isomerase. The activity of an enzyme can be “assayed,” either qualitatively (e.g., to determine if the activity is present) or quantitatively (e.g., to determine how much activity is present or kinetic and/or thermodynamic constants of the reaction).

A “kinase” is an enzyme that catalyzes the transfer of a phosphate group from one molecule to another. A “protein kinase” is a kinase that transfers a phosphate group to a protein, typically from a nucleotide such as ATP. A “tyrosine protein kinase” (or “tyrosine kinase”) transfers the phosphate to a tyrosine side chain (e.g., a particular tyrosine), while a “serine/threonine protein kinase” (“serine/threonine kinase”) transfers the phosphate to a serine or threonine side chain (e.g., a particular serine or threonine).

A “label” is a moiety that facilitates detection of a molecule. Exemplary labels include, but are not limited to, fluorescent, luminescent, magnetic, and/or colorimetric labels. Many labels are known in the art and commercially available and can be used in the context of the invention.

An “environmentally sensitive label” is a label whose signal changes when the environment of the label changes. For example, the fluorescence of an environmentally sensitive fluorescent label changes when the hydrophobicity, pH, and/or the like of the label's environment changes (e.g., upon binding of the molecule with which the label is associated to another molecule such that the label is transferred from an aqueous environment to a more hydrophobic environment at the molecular interface).

A “modulator” enhances or inhibits an activity of a protein (e.g., a catalytic activity of an enzyme), either partially or completely. An “activator” enhances the activity (whether moderately or strongly). An “inhibitor” inhibits the activity (e.g., an inhibitor of an enzyme attenuates the rate and/or efficiency of catalysis), whether moderately or strongly. A modulator can be, e.g., a small molecule, a polypeptide, a nucleic acid, etc.

The term “nucleic acid” encompasses any physical string of monomer units that can be corresponded to a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA in solution, such as 2′-O-methylated oligonucleotides), and the like. The nucleotides of the nucleic acid can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The nucleic acid can additionally comprise non-nucleotide elements such as labels, quenchers, blocking groups, or the like. A nucleic acid can be e.g., single-stranded or double-stranded. Unless otherwise indicated, a particular nucleic acid sequence of this invention encompasses complementary sequences, in addition to the sequence explicitly indicated.

A “phosphatase” is an enzyme that removes a phosphate group from a molecule. A “protein phosphatase” removes the phosphate group from an amino acid side chain in a protein. A “serine/threonine-specific protein phosphatase” removes the phosphate from a serine or threonine side chain (e.g., a particular serine or threonine), while a “tyrosine-specific protein phosphatase” removes the phosphate from a tyrosine side chain (e.g., a particular tyrosine).

A “polypeptide” is a polymer comprising two or more amino acid residues (e.g., a peptide or a protein). The polymer can additionally comprise non-amino acid elements such as labels, blocking groups, or the like and can optionally comprise modifications such as glycosylation or the like. The amino acid residues of the polypeptide can be natural or non-natural and can be unsubstituted, unmodified, substituted or modified.

A “protein transduction domain” is a polypeptide sequence that can mediate introduction of a covalently associated molecule into a cell. Protein transduction domains are typically short peptides (e.g., often less than about 16 residues). Example protein transduction domains have been derived from the HIV-1 protein TAT, the herpes simplex virus protein VP22, and the Drosophila protein antennapedia. Model protein transduction domains have also been designed.

A “ribozyme” is a catalytically active RNA molecule. It can operate in cis or trans.

A “subcellular delivery module” is a moiety that can mediate delivery and/or localization of an associated molecule to a particular subcellular location (e.g., a subcellular compartment, a membrane, and/or neighboring a particular macromolecule). The subcellular delivery module can be covalently or noncovalently associated with the molecule. Subcellular delivery modules include, e.g., peptide tags such as a nuclear localization signal or mitochondrial matrix-targeting signal.

“Uncaging energy” is energy that removes one or more caging groups from a caged molecule (or otherwise reverses the caging groups' blockage of the molecule's activity). As appropriate for the particular caging group(s), uncaging energy can be supplied, e.g., by light, sonication, a heat source, a magnetic field, or the like.

A “substrate” is a molecule on which an enzyme acts. The substrate is typically supplied in a first state on which the enzyme acts, converting it to a second state. The second state of the substrate is then typically released from the enzyme.

A “vector” is a compound or composition that includes or encodes one or more component of interest. Typical vectors include genetic vectors that include nucleic acids for the transmission of genetic information, as well as, optionally, accessory factors such as proteins, lipid membranes, and associated proteins (e.g., capsid or other structural proteins). An example of a type of genetic vector is a viral vector that can include proteins, polysaccharides, lipids, genetic material (nucleic acids, optionally including DNA and/or RNA) and the like. Another example of a genetic vector is a plasmid. In one typical configuration, the vector is a viral vector or a plasmid that encodes an enzyme or a sensor component (e.g., the enzyme or component is encoded in one or more open reading frame(s) of the vector). Many suitable vectors are well known and described, e.g., in Ausubel and Sambrook, both infra. An “expression vector” is a vector, such as a plasmid, which is capable of promoting expression as well as replication of a nucleic acid incorporated therein.

A “Dap residue” is an (L)-2,3-diaminopropionic acid residue.

A “Dab residue” is an (L)-2,4-diaminobutyric acid residue.

A variety of additional terms are defined or otherwise characterized herein.

### DETAILED DESCRIPTION

In one aspect, the invention provides a variety of sensors for detecting enzyme activity. In one class of embodiments, each sensor includes a substrate module and a detection module. The substrate module includes a substrate for the enzyme of interest and an environmentally sensitive label, whose signal changes when the environment of the label changes (e.g., an environmentally sensitive fluorophore whose signal changes with the hydrophobicity, pH, or the like of the label's surroundings). The detection module binds to the substrate module before or after the enzyme acts on the substrate and provides a different environment for the label (e.g., a relatively hydrophobic environment as compared to the label's environment when the substrate module is not bound to the detection module). In other embodiments, each sensor includes a polypeptide substrate and an environmentally sensitive or fluorescent label, typically, a label whose signal is altered upon phosphorylation or dephosphorylation of the substrate. Compositions including the sensors or components thereof and methods for using the sensors to detect enzyme activity and to screen for compounds affecting enzyme activity are described.

### Enzyme Sensors, Substrate Modules, and Detection Modules

A first general class of embodiments provides a composition including an enzyme and a sensor for detecting an activity of the enzyme. The sensor comprises a substrate module and a detection module. The substrate module includes a substrate for the enzyme, wherein the substrate is in a first state on which the enzyme can act, thereby converting the substrate to a second state, and an environmentally sensitive label. The detection module binds to the substrate module when the substrate is in the first state or when the substrate is in the second state. Binding of the detection module to the substrate module results in a change in signal from the label, e.g., since the label is in a different environment when the substrate module is bound to the detection module than when it is not bound to detection module. For example, binding of the substrate module to the detection module can result in a more hydrophobic or lipophilic environment, a different electrostatic environment, or the like for the label.

The substrate and detection modules can be part of a single molecule. More typically, however, the substrate module comprises a first molecule and the detection module comprises a second molecule. For example, the substrate module can comprise a first polypeptide and the detection module a second polypeptide. It is worth noting that the substrate module can comprise essentially any suitable substrate, for example, one or more of an amino acid, a polypeptide, a nitrogenous base, a nucleoside, a nucleotide, a nucleic acid, a carbohydrate, a lipid, or the like. The substrate is optionally a specific substrate (acted on only by a single type of catalytic molecule, e.g., under a defined set of reaction conditions), or a generic substrate (acted on by more than one member of a class of catalytic molecules). Similarly, the detection module can comprise essentially any molecule that can bind the first or second state of the substrate and can provide an appropriate environment for the environmentally sensitive label (e.g., a relatively hydrophobic environment), for example, a polypeptide, an aptamer, or the like.

The enzyme whose activity is to be detected can be essentially any enzyme. For example, the enzyme can be an oxidoreductase, transferase, hydrolase, lyase, ligase, or isomerase. In one embodiment, the enzyme catalyzes a posttranslational modification of a polypeptide, for example, phosphorylation, ubiquitination, sumoylation, glycosylation, prenylation, myristoylation, farnesylation, attachment of a fatty acid, attachment of a GPI anchor, acetylation, methylation, nucleotidylation (e.g., ADP-ribosylation), or the like. For example, the enzyme can be a transferase from any one of EC subclasses 2.1-2.9 (e.g., a glycosyltransferase, protein farnesyltransferase, or protein geranylgeranyltransferase), a ligase from any one of EC subclasses 6.1-6.6 (e.g., a ubiquitin transferase or ubiquitin-conjugating enzyme), or a hydrolase from any one of EC subclasses 3.1-3.13 (e.g., a phosphatase or glycosylase).

In one preferred class of embodiments, the enzyme is a protein kinase. In this class of embodiments, the substrate in the first state is unphosphorylated (not phosphorylated), and the substrate in the second state is phosphorylated. In some embodiments, the detection module binds to the substrate module when the substrate is in the first state; in other embodiments, the detection module binds to the substrate module when the substrate is in the second state (i.e., the detection module binds to the phosphorylated substrate).

In one class of embodiments, the protein kinase is a tyrosine protein kinase. The detection module is optionally, e.g., a polypeptide, an aptamer, or the like that recognizes the phosphorylated tyrosine substrate. For example, the detection module can include an SH2 domain, an FHA domain, a PTB (phosphotyrosine binding) domain, or an antibody. The substrate and detection modules optionally comprise distinct polypeptides.

In one exemplary class of embodiments, the substrate module includes a polypeptide comprising amino acid sequence X⁻⁴ X⁻³ X⁻² X⁻¹ Y⁰ X⁺¹ X⁺² X⁺³ X⁺⁴ X⁺⁵; where X⁻⁴, X⁻³, and X⁻² are independently selected from the group consisting of: D, E, and an amino acid residue comprising the environmentally sensitive label; X⁻¹ and X⁺³ are independently selected from the group consisting of: A, V, I, L, M, F, Y, W, and an amino acid residue comprising the environmentally sensitive label; X⁺¹, X⁺², X⁺⁴, and X⁺⁵ are independently selected from the group consisting of: an amino acid residue (e.g., a naturally occurring amino acid residue) and an amino acid residue comprising the environmentally sensitive label; and at least one of X⁻⁴, X⁻³, X⁻², X⁻¹, X⁺¹, X⁺², X⁺³, X⁺⁴, and X⁺⁵ is an amino acid residue comprising the environmentally sensitive label. For example, one of X⁺¹, X⁺², X⁺³, and X⁺⁴ can be an amino acid residue comprising the environmentally sensitive label. In one class of embodiments, the substrate module includes a polypeptide comprising an amino acid sequence selected from the group consisting of: EEEIYX⁺¹EIEA (SEQ ID NO:1) where X⁺¹ is an amino acid residue comprising the environmentally sensitive label, EEEIYGX⁺²IEA (SEQ ID NO:2) where X⁺² is an amino acid residue comprising the environmentally sensitive label, EEEIYGEX⁺³EA (SEQ ID NO:3) where X⁺³ is an amino acid residue comprising the environmentally sensitive label, and EEEIYGEIX⁺⁴A (SEQ ID NO:4) where X⁺⁴ is an amino acid residue comprising the environmentally sensitive label (e.g., a Dap, Dab, ornithine, lysine, cysteine, or homocysteine residue). For example, the substrate module can include a polypeptide comprising the amino acid sequence EEEIYGEIX⁺⁴A, where X⁺⁴ comprises a dapoxyl group attached to a Dab residue (SEQ ID NO:7); wherein the polypeptide substrate comprises a polypeptide comprising the amino acid sequence EEEIYGEX⁺³EA, where X⁺³ comprises a dapoxyl group attached to a Dab residue (SEQ ID NO:10); or wherein the polypeptide substrate comprises a polypeptide comprising the amino acid sequence EEEIYGEX⁺³EA, where X⁺³ comprises a dapoxyl group attached to a Dap residue (SEQ ID NO:11). An SH2 domain (e.g., an Lck SH2 domain), for example, is optionally used in the detection module. These and other exemplary kinase sensors are described in greater detail in Examples 1 and 2 below. The enzyme is optionally a tyrosine protein kinase (e.g., Src kinase) or a tyrosine-specific protein phosphatase. Y⁰ represents the tyrosine residue which is optionally phosphorylated by the kinase and/or dephosphorylated by the phosphatase. It will be evident that the label is optionally located at positions other than X⁺¹, X⁺², X⁺³, and X⁺⁴; for example, the polypeptide can comprise the amino acid sequence EEX⁻²IYGEIEA (SEQ ID NO:9), where X⁻² is an amino acid residue comprising the environmentally sensitive or fluorescent label (e.g., a Dap or Dab residue including pyrene).

In another class of embodiments, the protein kinase is a serine/threonine protein kinase. The detection module is optionally, e.g., a polypeptide, an aptamer, or the like that recognizes the phosphorylated serine and/or threonine substrate. For example, the detection module can include a 14-3-3, FHA, WD40, WW, Vhs, HprK, DSP, KIX, MH2, PKI, API3, ARM, cyclin, CDI, or GlgA domain, or an antibody. The substrate and detection modules optionally comprise distinct polypeptides. In alternative embodiments, the protein kinase can be, e.g., a histidine kinase, an asp/glu kinase, or an arginine kinase.

The phosphopeptide binding domains noted above, as well as other phosphopeptide binding domains, have been well described in the literature. For example, the specificity of various SH2 domains for sequences surrounding the phosphorylated tyrosine residue has been determined. See, e.g., a list of phosphopeptide binding domains at folding.cchmc.org/online/SEPdomaindatabase.htm; a list of protein interaction domains on the world wide web at mshri.on.ca/pawson/domains.html; a list of protein domains on the world wide web at cellsignal.com/reference/domain/index.asp, which includes consensus binding sites, exemplary peptide ligands, and exemplary binding partners, e.g., for SH-2, 14-3-3, PTB, and WW domains; Kuriyan and Cowburn (1997) “Modular peptide recognition domains in eukaryotic signaling” Annu. Rev. Biophys. Biomol. Struct. 26:259-288; Sharma et al. (2002) “Protein-protein interactions: Lessons learned” Curr. Med. Chem. —Anti-Cancer Agents 2:311-330; Pawson et al. (2001) “SH2 domains, interaction modules and cellular wiring” Trends Cell Biol. 11:504-11; Forman-Kay and Pawson (1999) “Diversity in protein recognition by PTB domains” Curr Opin Struct Biol. 9:690-5; and Fu et al. (2000) “14-3-3 Proteins: Structure, Function, and Regulation” Annual Review of Pharmacology and Toxicology 40:617-647. A large number of such domains from a variety of different proteins have been described, and others can readily be identified, e.g., through sequence alignment, structural comparison, and similar techniques, as is well known in the art. Common sequence repositories for known proteins include GenBank and Swiss-Prot, and other repositories can easily be identified by searching the internet. Similarly, antibodies against phosphotyrosine, phosphoserine, and/or phosphothreonine are well known in the art; many are commercially available, and others can be generated by established techniques. Other domains suitable for use as detection modules include, e.g., death domains, PDZ domains, and SH3 domains. The detection module is optionally a polypeptide (e.g., a recombinant polypeptide, e.g., based on fibronectin) selected for binding to the first or second state of the substrate by a technique such as phage display, mRNA display, or another in vitro or in vivo display and/or selection technique.

A large number of kinases and kinase substrates have been described in the art and can be adapted to the practice of the present invention. For example, the enzyme can be chosen from any of sub-sub-subclasses EC 2.7.1.1-2.7.1.156. In one class of embodiments, the kinase is a soluble (non-receptor) tyrosine kinase (for example, Abl, Arg, Blk, Bmx, Brk, BTK, Crk, Csk, DYRK1A, FAK, Fer, Fes/Fps, Fgr, Fyn, Hck, Itk, JAK, Lck, Lyn, MINK, Pyk, Src, Syk, Tec, Tyk, Yes, or ZAP-70), a receptor tyrosine kinase (for example, KIT, MET, KDR, EGFR, or an Eph receptor tyrosine kinase such as EphA1, EphA2, EphA3, EphA4, EphA5, EphA7, EphB1, EphB3, EphB4, or EphB6), a member of a MAP kinase pathway (for example, ARAF1, BRAF1, GRB2, MAPK1, MAP2K1, RASA1, SOS1, MAP2K2, and MAPK3; see, e.g., Cobb et al. (1996) Promega Notes Magazine 59:37-41), a member of an Akt signal pathway (e.g., PTEN, CDKN1A, GSK3B, PDPK1, CDKN1B, ILK, AKT1, PIK3CA, and CCND1), or a member of an EGFR signal pathway (e.g., EGFR, ARAF1, BRAF1, GRB2, MAPK1, MAP2K1, RASA1, SOS1, and MAP2K2). Exemplary kinases include, but are not limited to, Src; AMP-K, AMP-activated protein kinase; βARK, β adrenergic receptor kinase; CaMK, CaM-kinase, calmodulin-dependent protein kinase; cdc2 kinase, protein kinase expressed by CDC2 gene; cdk, cyclin dependent kinase; CK1, protein kinase CK1 (also termed casein kinase 1 or I); CK2, protein kinase CK2 (also termed casein kinase 2 or II); CSK, C-terminal Src protein kinase; GSK3, glycogen synthase kinase-3; HCR, heme controlled repressor, HRI; HMG-CoA reductase kinase A; insulin receptor kinase; MAP kinase, ERK, extracellular signal-regulated kinase; MAP kinase activated protein kinase 1; MAP kinase activated protein kinase 2; MLCK, myosin light chain kinase; Nek, NIMA-related kinase; NIMA, never in mitosis protein kinase; p70 s6k and p90 srk, 70 and 90 kDa kinases that phosphorylate s6 protein; PDHK, pyruvate dehydrogenase kinase; PhK, phosphorylase kinase; PKA, cAMP-dependent protein kinase A; PKB, protein kinase B; PKG, cGMP-dependent protein kinase, protein kinase G; PKR, RNA-dependent protein kinase, dSRNA-PK; PRK1, protein kinase C-related kinase 1; RAC; RhK, rhodopsin kinase; SNF-1 PK, sucrose non-fermenting protein kinase; Jun kinase, JNK; JNKKK; SrcN1, SrcN2, FynT, LynA, LynB, FGFR, TrkA, Flt3, and RSK.

Substrates for such kinases, including, e.g., protein substrates (e.g., another kinase, a histone, or myelin basic protein), amino acid polymers of random sequence (e.g., poly Glu/Tyr {4:1}), and/or polypeptide substrates with a defined amino acid sequence (e.g., chemically synthesized polypeptides; polypeptides including less than about 32 residues, less than about 20 residues, or less than about 15 residues; and polypeptides including between 7 and 15 residues), have been described in the art or can readily be determined by techniques known in art. See, e.g., Pinna and Ruzzene (1996) “How do protein kinases recognize their substrates?” Biochim Biophys Acta 1314:191-225. See, e.g., Example 2 for a list of exemplary kinases and polypeptide substrates.

In another preferred class of embodiments, the enzyme is a protein phosphatase. In this class of embodiments, the substrate in the first state is phosphorylated, and the substrate in the second state is unphosphorylated. In some embodiments, the detection module binds to the substrate module when the substrate is in the second state; in other embodiments, the detection module binds to the substrate module when the substrate is in the first state (i.e., the detection module binds to the phosphorylated substrate). Exemplary detection modules for the latter embodiments include those outlined above, e.g., SH2, PTB, 14-3-3, and other phosphoprotein binding domains, as well as antibodies and aptamers.

The phosphatase can be, e.g., a tyrosine-specific protein phosphatase (see, e.g., Alonso et al. (2004) “Protein Tyrosine Phosphatases in the Human Genome” Cell 117:699-711) or a serine/threonine-specific protein phosphatase (e.g., PP1, PP2A, PP2B, or PP2C). See also Example 2. It will be evident that a phosphorylated kinase sensor can serve as a phosphatase sensor (and vice versa). For example, exemplary PTP1B sensors can include a substrate module comprising a polypeptide comprising the amino acid sequence EEEIYGEIXA, where X comprises a dapoxyl group attached to a Dab residue (SEQ ID NO:7), comprising the amino acid sequence EEEIYGEXEA, where X comprises a dapoxyl group attached to a Dab residue (SEQ ID NO:10), or comprising the amino acid sequence EEEIYGEXEA, where X comprises a dapoxyl group attached to a Dap residue (SEQ ID NO:11), where the tyrosine residue is phosphorylated and where the detection module optionally comprises an SH2 domain (e.g., an Lck SH2 domain).

A variety of environmentally sensitive labels (e.g., fluorescent labels, magnetic labels, luminescent labels, and the like) are known in the art and can be adapted to the present invention. Further details can be found in the section entitled “Environmentally sensitive and fluorescent labels” below.

The substrate module optionally comprises a polypeptide comprising a Dap, Dab, ornithine, lysine, cysteine, or homocysteine residue (or essentially any other chemically reactive natural or unnatural amino acid derivative or residue) to which the environmentally sensitive label is attached. The label can be attached to the residue (e.g., before or after its incorporation into a polypeptide) by reacting a derivative of the label with a functional group on the residue's side chain, for example.

The sensors can be used in biochemical assays of enzyme activity, to detect enzyme activity inside cells and/or organisms, or the like. Thus, the composition optionally includes a cell lysate or a cell, e.g., a cell comprising the sensor, a cell comprising the enzyme, or a cell comprising the enzyme and the sensor.

The substrate module is optionally associated with a cellular delivery module that can mediate introduction of the substrate module into a cell, e.g., a lipid or polypeptide such as those described in the section entitled “In vivo and in vitro cellular delivery” below. Similarly, the detection module is optionally associated with a cellular delivery module that can mediate introduction of the detection module into the cell. Alternatively, the detection module can be endogenous to the cell. For example, the detection module can be expressed from the cell's genome, from a nucleic acid construct transiently or stably transfected into the cell, or the like.

In one class of embodiments, the sensor is caged such that the enzyme can not act upon the substrate until the sensor is uncaged, for example, by removal of a photolabile caging group. Thus, in one class of embodiments, the sensor comprises one or more caging groups associated with the substrate module. The caging groups inhibit the enzyme from acting upon the substrate, e.g., by at least about 75%, at least about 90%, at least about 95%, or at least about 98%, as compared to the substrate in the absence of the one or more caging groups. Preferably, the one or more caging groups prevent the enzyme from acting upon the substrate. Typically, removal of, or an induced conformational change in, the one or more caging groups permits the enzyme to act upon the substrate. The one or more caging groups associated with the substrate module can be covalently or non-covalently attached to the substrate module. In a preferred aspect, the one or more caging groups are photoactivatable (e.g., photolabile). Caging groups are described in greater detail below, in the section entitled “Caging groups”.

Caging of the sensor permits initiation of the reaction between the enzyme and the substrate within the sensor to be controlled, temporally and/or spatially. Similar or additional control of the reaction can be obtained through use of other caged reagents, for example, caged nucleotides (e.g., caged ATP), caged metal ions, caged chelating agents (e.g., caged EDTA or EGTA), caged activators or inhibitors, and the like. See, e.g., US patent application publication 2004/0166553 by Nguyen et al. entitled “Caged sensors, regulators and compounds and uses thereof.” It will be evident that essentially any of the features noted herein can be used in combination; as just one example, a composition including a caged, fluorescently labeled sensor located in a cell is a feature of the invention.

The sensor can be used to study the effects of activators and inhibitors (known and potential) on the enzyme's activity. Thus, the composition optionally includes a modulator or potential modulator of the activity of the enzyme.

Two or more enzyme activities can be monitored simultaneously or sequentially, if desired, by including in the composition a second sensor. The second sensor can comprise a second substrate module including a second substrate for a second enzyme and a second environmentally sensitive label, whose signal is detectably different from that of the first sensor's label upon binding to a second detection module, or the second sensor can comprise a polypeptide including an environmentally sensitive or fluorescent label (such as the polypeptides described below in the section entitled “Sensors including environmentally sensitive or fluorescent labels”).

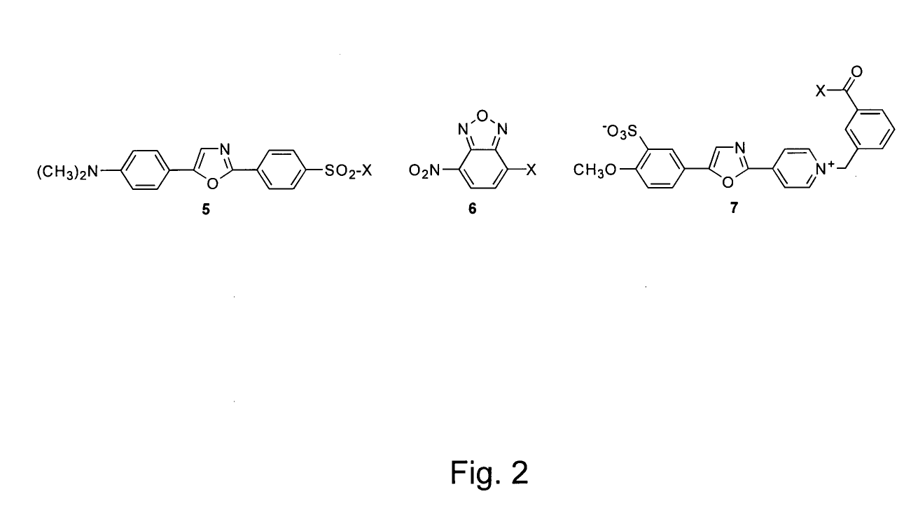

Other embodiments provide compositions including components of enzyme sensors (e.g., substrate and/or detection modules) and/or nucleic acids encoding such components. Thus, a second general class of embodiments provides a composition comprising a polypeptide substrate that includes an environmentally sensitive label and a polypeptide comprising amino acid sequence X⁻⁴ X⁻³ X⁻² X⁻¹ Y⁰ X⁺¹ X⁺² X⁺³ X⁺⁴ X⁺⁵; where X⁻⁴, X⁻³, and X⁻² are independently selected from the group consisting of: D, E, and an amino acid residue comprising the environmentally sensitive label; X⁻¹ and X⁺³ are independently selected from the group consisting of: A, V, I, L, M, F, Y, W, and an amino acid residue comprising the environmentally sensitive label; X⁺¹, X⁺², X⁺⁴, and X⁺⁵ are independently selected from the group consisting of: an amino acid residue and an amino acid residue comprising the environmentally sensitive label; and at least one of X⁻⁴, X⁻³, X⁻², X⁻¹, X⁺¹, X⁺², X⁺³, X⁺⁴, and X⁺⁵ is an amino acid residue comprising the environmentally sensitive label. For example, one of X⁺¹, X⁺², X⁺³, and X⁺⁴ can be an amino acid residue comprising the environmentally sensitive label. In one class of embodiments, the polypeptide substrate includes a polypeptide comprising an amino acid sequence selected from the group consisting of: EEEIYX⁺¹EIEA (SEQ ID NO:1) where X⁺¹ is an amino acid residue comprising the environmentally sensitive label, EEEIYGX⁺²IEA (SEQ ID NO:2) where X⁺² is an amino acid residue comprising the environmentally sensitive label, EEEIYGEX⁺³EA (SEQ ID NO:3) where X⁺³ is an amino acid residue comprising the environmentally sensitive label, and EEEIYGEIX⁺⁴A (SEQ ID NO:4) where X⁺⁴ is an amino acid residue comprising the environmentally sensitive label (e.g., a Dap, Dab, ornithine, lysine, cysteine, or homocysteine residue). For example, the polypeptide substrate can include a polypeptide comprising the amino acid sequence EEEIYGEIX⁺⁴A, where X⁺⁴ comprises a dapoxyl group attached to a Dab residue (SEQ ID NO:7); wherein the polypeptide substrate comprises a polypeptide comprising the amino acid sequence EEEIYGEX⁺³EA, where X⁺³ comprises a dapoxyl group attached to a Dab residue (SEQ ID NO:10); or wherein the polypeptide substrate comprises a polypeptide comprising the amino acid sequence EEEIYGEX⁺³EA, where X⁺³ comprises a dapoxyl group attached to a Dap residue (SEQ ID NO:11). The label can include a fluorophore selected from 5-7 (FIG. 2). These and other exemplary kinase substrate modules are described in greater detail in Example 1 below.

The composition optionally also includes a second polypeptide comprising an SH2 domain (e.g., an Lck SH2 domain), a PTB domain, or an antibody. Similarly, the composition optionally also includes a kinase (e.g., Src), a cell, or a cell lysate. The tyrosine residue in the polypeptide substrate is optionally phosphorylated, and the composition can include a protein phosphatase.

A third general class of embodiments provides a composition useful, e.g., in in-cell assays in which the enzyme to be detected and/or the detection module is expressed (e.g., overexpressed) in a cell or cell line. The composition includes a substrate module that comprises a substrate for an enzyme, wherein the substrate is in a first state on which the enzyme can act, thereby converting the substrate to a second state, and an environmentally sensitive label. The composition also includes a nucleic acid encoding the enzyme, a nucleic acid encoding a detection module (which detection module binds to the substrate module when the substrate is in the first state, or which detection module binds to the substrate module when the substrate is in the second state, wherein binding of the detection module to the substrate module results in a change in signal from the label), or both. In embodiments in which the composition includes both a nucleic acid encoding the enzyme and a nucleic acid encoding the detection module, the nucleic acids can be part of the same molecule (e.g., located on the same expression vector) or different molecules (e.g., separate vectors).

Essentially all of the features noted above apply to this class of embodiments as well, as relevant: for example, with respect to type of enzyme, exemplary substrate and detection modules, fluorescent labels, use of caging groups, use of cellular delivery modules, and/or the like.

Thus, for example, in one preferred class of embodiments, the enzyme is a protein kinase. In this class of embodiments, the substrate in the first state is unphosphorylated, and the substrate in the second state is phosphorylated. In some embodiments, the detection module binds to the substrate module when the substrate is in the first state; in other embodiments, the detection module binds to the substrate module when the substrate is in the second state (i.e., the detection module binds to the phosphorylated substrate).

In one class of embodiments, the protein kinase is a tyrosine protein kinase. The detection module is optionally, e.g., a polypeptide, an aptamer, or the like that recognizes the phosphorylated tyrosine substrate. For example, the detection module can include an SH2 domain, an FHA domain, a PTB (phosphotyrosine binding) domain, or an antibody. The substrate and detection modules optionally comprise distinct polypeptides.

In one exemplary class of embodiments, the enzyme is a tyrosine protein kinase (e.g., Src kinase), and the substrate module includes a polypeptide comprising amino acid sequence X⁻⁴ X⁻³ X⁻² X⁻¹ Y⁰ X⁺¹ X⁺² X⁺³ X⁺⁴ X⁺⁵; where X⁻⁴, X⁻³, and X⁻² are independently selected from the group consisting of: D, E, and an amino acid residue comprising the environmentally sensitive label; X⁻¹ and X⁺³ are independently selected from the group consisting of: A, V, I, L, M, F, Y, W, and an amino acid residue comprising the environmentally sensitive label; X⁺¹, X⁺², X⁺⁴, and X⁺⁵ are independently selected from the group consisting of: an amino acid residue (e.g., a naturally occurring amino acid residue) and an amino acid residue comprising the environmentally sensitive label; and at least one of X⁻⁴, X⁻³, X⁻², X⁻¹, X⁺¹, X⁺², X⁺³, X⁺⁴, and X⁺⁵ is an amino acid residue comprising the environmentally sensitive label. For example, one of X⁺¹, X⁺², X⁺³, and X⁺⁴ can be an amino acid residue comprising the environmentally sensitive label. In one class of embodiments, the substrate module includes a polypeptide comprising an amino acid sequence selected from the group consisting of: EEEIYX⁺¹EIEA (SEQ ID NO:1) where X⁺¹ is an amino acid residue comprising the environmentally sensitive label, EEEIYGX⁺²IEA (SEQ ID NO:2) where X⁺² is an amino acid residue comprising the environmentally sensitive label, EEEIYGEX⁺³EA (SEQ ID NO:3) where X⁺³ is an amino acid residue comprising the environmentally sensitive label, and EEEIYGEIX⁺⁴A (SEQ ID NO:4) where X⁺⁴ is an amino acid residue comprising the environmentally sensitive label (e.g., a Dap, Dab, ornithine, lysine, cysteine, or homocysteine residue). For example, the substrate module can include a polypeptide comprising the amino acid sequence EEEIYGEIX⁺⁴A, where X⁺⁴ comprises a dapoxyl group attached to a Dab residue (SEQ ID NO:7); wherein the polypeptide substrate comprises a polypeptide comprising the amino acid sequence EEEIYGEX⁺³EA, where X⁺³ comprises a dapoxyl group attached to a Dab residue (SEQ ID NO:10); or wherein the polypeptide substrate comprises a polypeptide comprising the amino acid sequence EEEIYGEX⁺³EA, where X⁺³ comprises a dapoxyl group attached to a Dap residue (SEQ ID NO:11). An SH2 domain (e.g., an Lck SH2 domain), for example, is optionally used in the detection module. These and other exemplary substrate modules are described in greater detail in Examples 1 and 2 below.

In another class of embodiments, the protein kinase is a serine/threonine protein kinase. The detection module is optionally, e.g., a polypeptide, an aptamer, or the like that recognizes the phosphorylated serine and/or threonine substrate. For example, the detection module can include a 14-3-3, FHA, WD40, WW, Vhs, HprK, DSP, KIX, MH2, PKI, API3, ARM, cyclin, CDI, or GlgA domain, or an antibody. The substrate and detection modules optionally comprise distinct polypeptides. In alternative embodiments, the protein kinase can be, e.g., a histidine kinase, an asp/glu kinase, or an arginine kinase.

In another preferred class of embodiments, the enzyme is a protein phosphatase. In this class of embodiments, the substrate in the first state is phosphorylated, and the substrate in the second state is unphosphorylated. In some embodiments, the detection module binds to the substrate module when the substrate is in the second state; in other embodiments, the detection module binds to the substrate module when the substrate is in the first state (i.e., the detection module binds to the phosphorylated substrate). Exemplary detection modules for the latter embodiments include those outlined above, e.g., SH2, PTB, 14-3-3, and other phosphoprotein binding domains, as well as antibodies and aptamers.

A variety of environmentally sensitive labels (e.g., fluorescent labels, magnetic labels, luminescent labels, and the like) are known in the art and can be adapted to the present invention. Further details can be found, e.g., in the section entitled “Environmentally sensitive and fluorescent labels” below.

The substrate module is optionally associated with a cellular delivery module that can mediate introduction of the substrate module into a cell, e.g., a lipid or polypeptide such as those described in the section entitled “In vivo and in vitro cellular delivery” below.

Similarly, the substrate module is optionally caged such that the enzyme can not act upon the substrate until the substrate module is uncaged, for example, by removal of a photolabile caging group. Thus, in one class of embodiments, the composition comprises one or more caging groups associated with the substrate module. The caging groups inhibit the enzyme from acting upon the substrate, e.g., by at least about 75%, at least about 90%, at least about 95%, or at least about 98%, as compared to the substrate in the absence of the one or more caging groups. Preferably, the one or more caging groups prevent the enzyme from acting upon the substrate. Typically, removal of, or an induced conformational change in, the one or more caging groups permits the enzyme to act upon the substrate. The one or more caging groups associated with the substrate module can be covalently or non-covalently attached to the substrate module. In a preferred aspect, the one or more caging groups are photoactivatable (e.g., photolabile). Caging groups are described in greater detail below, in the section entitled “Caging groups”.

It is worth noting that the composition optionally includes a cell, e.g., a cell comprising the substrate module, the nucleic acid encoding the enzyme, the nucleic acid encoding the detection module, the enzyme (e.g., expressed from the corresponding nucleic acid), and/or the detection module (e.g., expressed from the corresponding nucleic acid).

Sensors with Environmentally Sensitive or Fluorescent Labels

As described above, one aspect of the invention provides sensors that include a substrate module and a detection module. Another aspect of the invention, however, provides sensors that function even the absence of any detection module. Such sensors include a fluorescent label or an environmentally sensitive label that responds to local environmental changes triggered directly by modification (e.g., phosphorylation) of a substrate, rather than indirectly by binding of a detection module to the modified (e.g., phosphorylated) substrate.

One general class of embodiments provides a composition that includes a polypeptide (typically, a polypeptide substrate) comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label; X⁻¹ and X⁺³ are independently selected from the group consisting of: A, V, I, L, M, F, Y, W, and an amino acid residue comprising the environmentally sensitive or fluorescent label; and X⁺¹, X⁺², X⁺⁴, and X⁺⁵ are independently selected from the group consisting of: an amino acid residue and an amino acid residue comprising the environmentally sensitive or fluorescent label. At least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label.

In one class of embodiments, one of X⁺¹, X⁺², X⁺³, and X⁺⁴ is an amino acid residue comprising the environmentally sensitive or fluorescent label. For example, the polypeptide can comprise an amino acid sequence selected from the group consisting of: EEEIYX⁺¹EIEA (SEQ ID NO:1) where X⁺¹ is an amino acid residue comprising the environmentally sensitive or fluorescent label, EEEIYGX⁺²IEA (SEQ ID NO:2) where X⁺² is an amino acid residue comprising the environmentally sensitive or fluorescent label, EEEIYGEX⁺³EA (SEQ ID NO:3) where X⁺³ is an amino acid residue comprising the environmentally sensitive or fluorescent label, and EEEIYGEIX⁺⁴A (SEQ ID NO:4) where X⁺⁴ is an amino acid residue comprising the environmentally sensitive or fluorescent label. X⁺¹, X⁺², X⁺³, or X⁺⁴ optionally comprises a Dap, Dab, ornithine, lysine, cysteine, or homocysteine residue, or essentially any other residue to which the label can be attached. Thus, for example, the polypeptide optionally comprises the amino acid sequence EEEIYGEIX⁺⁴A, where X⁺⁴ comprises a dapoxyl group attached to a Dab residue (SEQ ID NO:7), the amino acid sequence EEEIYGEX⁺³EA, where X⁺³ comprises a dapoxyl group attached to a Dab residue (SEQ ID NO:10), or the amino acid sequence EEEIYGEX⁺³EA, where X⁺³ comprises a dapoxyl group attached to a Dap residue (SEQ ID NO:11).

In one class of embodiments, one of X⁻² and X⁺³ is an amino acid residue comprising the environmentally sensitive or fluorescent label. For example, the polypeptide optionally comprises an amino acid sequence selected from the group consisting of: EEX⁻²IYGEIEA (SEQ ID NO:9), where X⁻² is an amino acid residue comprising the environmentally sensitive or fluorescent label, and EEEIYGEX⁺³EA (SEQ ID NO:3), where X⁺³ is an amino acid residue comprising the environmentally sensitive or fluorescent label. X⁻² or X⁺³ optionally comprises a Dap, Dab, ornithine, lysine, cysteine, or homocysteine residue, or essentially any other residue to which the label can be attached. Thus, for example, the polypeptide can comprise the amino acid sequence EEX⁻²IYGEIEA, where X⁻² comprises pyrene attached to a Dab residue (SEQ ID NO:12), the amino acid sequence EEEIYGEX⁺³EA, where X⁺³ comprises pyrene attached to a Dab residue (SEQ ID NO:13), the amino acid sequence EEEIYGEX⁺³EA, where X⁺³ comprises pyrene attached to a Dap residue (SEQ ID NO:14), the amino acid sequence EEX⁻²IYGEIEA, where X⁻² comprises Cascade Yellow attached to a Dab residue (SEQ ID NO:15), the amino acid sequence EEX⁻²IYGEIEA, where X⁻² comprises 2,7-difluorofluorescein (Oregon Green™ 488-X) attached to a Dab residue (SEQ ID NO:17), the amino acid sequence EEEIYGEX⁺³EA, where X⁺³ comprises 2,7-difluorofluorescein (Oregon Green™ 488-X) attached to a Dap residue (SEQ ID NO:18), the amino acid sequence EEX⁻²IYGEIEA, where X⁻² comprises Cascade Blue™ attached to a Dab residue (SEQ ID NO:19), or the amino acid sequence EEEIYGEX⁺³EA, where X⁺³ comprises Cascade Blue™ attached to a Dap residue (SEQ ID NO:20). These and other exemplary sensors are described in greater detail in Examples 2, 3, and 4 below.

It will be evident that the label is optionally attached at positions other than, or in addition to, X⁻², X⁺¹, X⁺², X⁺³, and X⁺⁴ and/or that the polypeptide optionally comprises other amino acid sequences. The above polypeptides are provided purely by way of example.

In one class of embodiments, the label is a fluorescent label. The fluorescent label is optionally also environmentally sensitive; in other embodiments, the fluorescent label is not environmentally sensitive. A variety of environmentally sensitive and/or fluorescent labels (including, e.g., pyrene, NBD, Cascade Yellow, dapoxyl, 2,7-difluorofluorescein (Oregon Green™ 488-X), 7-diethylaminocoumarin-3-carboxylic acid, 5-carboxyfluorescein, Texas Red™-X, Marina Blue™, Pacific Blue™, Cascade Blue™, bimane, 2-anthracenesulfonyl, dansyl, Alexa Fluor 430, PyMPO, 5-carboxytetramethylrhodamine (5-TAMRA), 6-carboxytetramethylrhodamine (6-TAMRA), BODIPY FL, and 3,4,9,10-perylene-tetracarboxylic acid) are known in the art and can be adapted to the practice of the present invention. Further details can be found below, in the section entitled “Environmentally sensitive and fluorescent labels.”

In one preferred class of embodiments, the composition further comprises a tyrosine protein kinase, typically, a kinase for which the polypeptide is, or is suspected to be, a substrate. Exemplary kinases include, but are not limited to, Src, SrcN1, SrcN2, FynT, Fgr, Lck, Yes, LynA, LynB, Hck, Abl, Csk, Fes/Fps, FGFR, TrkA, and Flt3. In another preferred class of embodiments, the composition further comprises a protein phosphatase, typically, a tyrosine-specific protein phosphatase for which the polypeptide is, or is suspected to be, a substrate.

The tyrosine at the phosphorylation site, Y⁰, optionally comprises a free hydroxyl group (i.e., is unphosphorylated), or is optionally a phosphorylated tyrosine residue.

Preferably, phosphorylation (or, correspondingly, dephosphorylation) of Y⁰ results in a change in signal from the label (e.g., a change in fluorescence emission intensity, wavelength, and/or duration from a fluorescent label). In one class of embodiments, the change in signal depends on the presence of a detection module. Thus, in this class of embodiments, the composition optionally also includes a second polypeptide comprising a detection module such as an SH2 domain, a PTB domain, or an antibody. Binding of the second polypeptide to the phosphorylated substrate leads to the change in signal. In a preferred class of embodiments, however, no detection module is required for the change in signal to result from phosphorylation (or dephosphorylation) of Y⁰. In this class of embodiments, no detection module, second polypeptide, or the like need be present in the composition. In this class of embodiments, for example, the change in signal can result from a phosphorylation-induced change in the local environment of an environmentally sensitive label, from disruption of an interaction between a fluorescent or environmentally sensitive label and Y⁰ upon phosphorylation of Y⁰, and/or the like.

Essentially all of the features noted above apply to this class of embodiments as well, as relevant; for example, with respect to type of kinase or phosphatase, inclusion of a second sensor in the composition, use of cellular delivery modules, inclusion of a nucleic acid encoding a kinase or phosphatase whose activity is to be detected, inclusion of a modulator or potential modulator of the activity of the enzyme, and/or the like.

Thus, for example, the sensors can be used in biochemical assays of enzyme activity, to detect enzyme activity inside cells and/or organisms, or the like. Thus, the composition optionally includes a cell lysate or a cell, e.g., a cell comprising the sensor, a cell comprising the enzyme, or a cell comprising the enzyme and the sensor.

As another example, the sensor is optionally caged, such that an enzyme (e.g., a tyrosine kinase or phosphatase) can not act on (phosphorylate or dephosphorylate) the polypeptide until it is uncaged, for example, by removal of a photolabile caging group. Thus, in one class of embodiments, the composition comprises one or more caging groups associated with the polypeptide. The caging groups inhibit an enzyme from acting upon the polypeptide, e.g., by at least about 75%, at least about 90%, at least about 95%, or at least about 98%, as compared to the polypeptide in the absence of the one or more caging groups. Preferably, the one or more caging groups prevent the enzyme from acting upon the polypeptide. Typically, removal of, or an induced conformational change in, the one or more caging groups permits the enzyme to act upon the polypeptide. The one or more caging groups associated with the polypeptide can be covalently or non-covalently attached to the polypeptide. For example, a single caging group can be covalently attached to the Y⁰ side chain (e.g., a photolabile caging group can be attached to the oxygen of the tyrosine hydroxyl group, preventing phosphorylation of the polypeptide by a tyrosine kinase until the caging group is removed, or to the phosphate group on a phosphorylated tyrosine, preventing dephosphorylation by a phosphatase until the caging group is removed). In a preferred aspect, the one or more caging groups are photoactivatable (e.g., photolabile). Caging groups are described in greater detail below, in the section entitled “Caging groups”.

In one aspect, the invention provides kinase or phosphatase sensors including a label whose interaction with the residue that is phosphorylated is altered upon phosphorylation or dephosphorylation of the residue, leading to a change in signal from the label. Thus, another general class of embodiments provides a composition that includes a polypeptide (typically, a polypeptide substrate) comprising an environmentally sensitive or fluorescent label. The polypeptide comprises a tyrosine residue, and when the tyrosine is unphosphorylated, it engages in an interaction with the label. This interaction is at least partially disrupted (e.g., completely disrupted) when the tyrosine is phosphorylated, such that a signal from the label changes upon phosphorylation or dephosphorylation of the tyrosine.

As noted, when the tyrosine is unphosphorylated, it engages in an interaction with the label. Thus, typically, one or more atoms of the tyrosine engage in electrostatic, van der Waals, hydrophobic, and/or similar noncovalent interactions with one or more atoms of the label when the tyrosine is unphosphorylated. It will be evident that there are a variety of ways in which the tyrosine and the label can interact. For example, in one class of embodiments, the environmentally sensitive or fluorescent label comprises an aromatic ring; when the tyrosine is unphosphorylated, it engages in an interaction with the aromatic ring of the label, and the interaction is at least partially disrupted when the tyrosine is phosphorylated. For example, when the tyrosine is unphosphorylated, it can engage in a π-π stacking interaction or an edge-face interaction with the aromatic ring of the label. As a similar example, when the tyrosine is unphosphorylated, it can engage in a cation-π interaction with the label. Optionally, when the tyrosine is phosphorylated, it does not engage in the π-π stacking, edge-face, or cation-π interaction with the label.

Cation-π interactions, π-π stacking (which is also known as face-to-face offset stacking), and edge-face interactions have been well described in the scientific literature. The existence of, and changes in (e.g., disruption of), such interactions can be detected by techniques such as nuclear magnetic resonance (NMR) spectroscopy, for example. The aromatic region of the NMR spectrum of an unphosphorylated polypeptide in which the tyrosine interacts with a cation or an aromatic ring in the label typically exhibits chemical shifts and NOEs characteristic of a cation-π, π-π stacking, or edge-face interaction if such an interaction is present; the pattern of chemical shifts and NOEs alters when the tyrosine is phosphorylated if the phosphorylation disrupts the interaction. Additional details on aromatic interactions and detection of such interactions by NMR is available, e.g., in Hunter et al. (2001) “Aromatic interactions” J. Chem. Soc., Perkin Trans. 2:651-669, Tatko and Waters (2002) “Selective aromatic interactions in β-hairpin peptides” J. Am. Chem. Soc. 124:9372-9373, Tatko and Waters (2003) “The geometry and efficacy of cation-π interactions in a diagonal position of a designed β-hairpin” Protein Science 12:2443-2452, Tatko (2002) “Aromatic interactions in biological systems” American Chemical Society Division of Organic Chemistry fellowship essay, available on the internet at organicdivision.org/essays_{—}2002/tatko.pdf, Ma and Dougherty (1997) “The cation-π interaction” Chem. Rev. 97:1303-1324, Dougherty (1996) “Cation-π interactions in chemistry and biology: A new view of benzene, Phe, Tyr, and Trp” Science 271:163-168, and references therein, as well as in Example 3 below.

The polypeptide is typically a polypeptide substrate, e.g., for at least one kinase and/or phosphatase. In one preferred class of embodiments, the composition further comprises a tyrosine protein kinase, typically, a kinase for which the polypeptide is, or is suspected to be, a substrate. Exemplary kinases include, but are not limited to, Src, SrcN1, SrcN2, FynT, Fgr, Lck, Yes, LynA, LynB, Hck, Abl, Csk, Fes/Fps, FGFR, TrkA, and Flt3. In another preferred class of embodiments, the composition further comprises a protein phosphatase, typically, a tyrosine-specific protein phosphatase for which the polypeptide is, or is suspected to be, a substrate.

In one class of embodiments, the label is a fluorescent label. The fluorescent label is optionally also environmentally sensitive; in other embodiments, the fluorescent label is not environmentally sensitive. A variety of environmentally sensitive and/or fluorescent labels are known in the art and can be adapted to the practice of the present invention. Further details can be found in the section entitled “Environmentally sensitive and fluorescent labels” below.

In one exemplary class of embodiments, the polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label; X⁻¹ and X⁺³ are independently selected from the group consisting of: A, V, I, L, M, F, Y, W, and an amino acid residue comprising the environmentally sensitive or fluorescent label; and X⁺¹, X⁺², X⁺⁴, and X⁺⁵ are independently selected from the group consisting of: an amino acid residue and an amino acid residue comprising the environmentally sensitive or fluorescent label. At least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label.

In one class of embodiments, one of X⁻² and X⁺³ is an amino acid residue comprising the environmentally sensitive or fluorescent label. For example, the polypeptide optionally comprises an amino acid sequence selected from the group consisting of: EEX⁻²IYGEIEA (SEQ ID NO:9), where X⁻² is an amino acid residue comprising the environmentally sensitive or fluorescent label, and EEEIYGEX⁺³EA (SEQ ID NO:3), where X⁺³ is an amino acid residue comprising the environmentally sensitive or fluorescent label. X⁻² or X⁺³ optionally comprises a Dap, Dab, ornithine, lysine, cysteine, or homocysteine residue, or essentially any other residue to which the label can be attached. Thus, for example, the polypeptide can comprise the amino acid sequence EEX⁻²IYGEIEA, where X⁻² comprises pyrene attached to a Dab residue (SEQ ID NO:12), the amino acid sequence EEEIYGEX⁺³EA, where X⁺³ comprises pyrene attached to a Dab residue (SEQ ID NO:13), the amino acid sequence EEEIYGEX⁺³EA, where X⁺³ comprises pyrene attached to a Dap residue (SEQ ID NO:14), the amino acid sequence EEX⁻²IYGEIEA, where X⁻² comprises Cascade Yellow attached to a Dab residue (SEQ ID NO:15), the amino acid sequence EEX⁻²IYGEIEA, where X⁻² comprises 2,7-difluorofluorescein (Oregon Green™ 488-X) attached to a Dab residue (SEQ ID NO:17), the amino acid sequence EEEIYGEX⁺³EA, where X⁺³ comprises 2,7-difluorofluorescein (Oregon Green™ 488-X) attached to a Dap residue (SEQ ID NO:18), the amino acid sequence EEX⁻²IYGEIEA, where X⁻² comprises Cascade Blue™ attached to a Dab residue (SEQ ID NO:19), or the amino acid sequence EEEIYGEX⁺³EA, where X⁺³ comprises Cascade Blue™ attached to a Dap residue (SEQ ID NO:20). It will be evident that any of a variety of labels can be employed, that the label is optionally attached at positions other than, or in addition to, X⁻² and X⁺³, and/or that the polypeptide optionally comprises other amino acid sequences; the above polypeptides are provided purely by way of example.

Essentially all of the features noted above apply to this class of embodiments as well, as relevant; for example, with respect to type of kinase or phosphatase, inclusion of a second sensor in the composition, use of cellular delivery modules, inclusion of a nucleic acid encoding a kinase or phosphatase whose activity is to be detected, inclusion of a modulator or potential modulator of the activity of the enzyme, caging of the polypeptide, inclusion of a cell or cell lysate, and/or the like.

In one aspect, the invention provides kinase or phosphatase sensors including a polypeptide substrate and a label that is located at a defined position with respect to the phosphorylation site in the substrate. For example, the label can be located at amino acid position −4, −3, −2, −1, +1, +2, +3, +4, and/or +5 with respect to the phosphorylation site. Thus, one general class of embodiments provides a composition that includes a polypeptide substrate for a protein tyrosine kinase or a tyrosine-specific protein phosphatase. The polypeptide substrate comprises an environmentally sensitive or fluorescent label, which is located at amino acid position −2 or +3 with respect to the phosphorylation site (the tyrosine that is phosphorylated by the kinase or dephosphorylated by the phosphatase) within the polypeptide substrate. It will be evident that the substrate optionally comprises one or more additional amino acid residues N- and/or C-terminal of the residues at positions −2 and/or +3.

In a preferred class of embodiments, phosphorylation or dephosphorylation of the substrate at the phosphorylation site results in a change in signal from the label. In one class of embodiments, the label is a fluorescent label. The fluorescent label is optionally also environmentally sensitive; in other embodiments, the fluorescent label is not environmentally sensitive. A variety of environmentally sensitive and/or fluorescent labels are known in the art and can be adapted to the practice of the present invention. Further details can be found in the section entitled “Environmentally sensitive and fluorescent labels” below.

In one exemplary class of embodiments, the polypeptide substrate comprises a polypeptide having amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label; X⁻¹ and X⁺³ are independently selected from the group consisting of: A, V, I, L, M, F, Y, W, and an amino acid residue comprising the environmentally sensitive or fluorescent label; and X⁺¹, X⁺², X⁺⁴, and X⁺⁵ are independently selected from the group consisting of: an amino acid residue and an amino acid residue comprising the environmentally sensitive or fluorescent label. At least one of X⁻² and X⁺³ is an amino acid residue comprising the environmentally sensitive or fluorescent label. For example, the polypeptide optionally comprises an amino acid sequence selected from the group consisting of: EEX²IYGEIEA (SEQ ID NO:9), where X⁻² is an amino acid residue comprising the environmentally sensitive or fluorescent label, and EEEIYGEX⁺³EA (SEQ ID NO:3), where X⁺³ is an amino acid residue comprising the environmentally sensitive or fluorescent label. X⁻² or X⁺³ optionally comprises a Dap, Dab, ornithine, lysine, cysteine, or homocysteine residue, or essentially any other residue to which the label can be attached. Thus, for example, the polypeptide can comprise the amino acid sequence EEX⁻²IYGEIEA, where X⁻² comprises pyrene attached to a Dab residue (SEQ ID NO:12), the amino acid sequence EEEIYGEX⁺³EA, where X⁺³ comprises pyrene attached to a Dab residue (SEQ ID NO:13), the amino acid sequence EEEIYGEX⁺³EA, where X⁺³ comprises pyrene attached to a Dap residue (SEQ ID NO:14), the amino acid sequence EEX⁻²IYGEIEA, where X⁻² comprises Cascade Yellow attached to a Dab residue (SEQ ID NO:15), the amino acid sequence EEX²IYGEIEA, where X⁻² comprises 2,7-difluorofluorescein (Oregon Green™ 488-X) attached to a Dab residue (SEQ ID NO:17), the amino acid sequence EEEIYGEX⁺³EA, where X⁺³ comprises 2,7-difluorofluorescein (Oregon Green™ 488-X) attached to a Dap residue (SEQ ID NO:18), the amino acid sequence EEX²IYGEIEA, where X⁻² comprises Cascade Blue™ attached to a Dab residue (SEQ ID NO:19), or the amino acid sequence EEEIYGEX⁺³EA, where X⁺³ comprises Cascade Blue™ attached to a Dap residue (SEQ ID NO:20). It will be evident that any of a variety of labels can optionally be employed, that the label is optionally attached at positions other than, or in addition to, X⁻² and X⁺³, and/or that the polypeptide optionally comprises other amino acid sequences. The above polypeptides are provided purely by way of example.

Essentially all of the features noted above apply to this class of embodiments as well, as relevant; for example, with respect to inclusion and type of kinase or phosphatase, use of cellular delivery modules, inclusion of a nucleic acid encoding a kinase or phosphatase whose activity is to be detected, inclusion of a modulator or potential modulator of the activity of the enzyme, caging of the polypeptide, inclusion of a cell or cell lysate, and/or the like.

### Methods for Detecting Enzyme Activity

In one aspect, the invention provides methods for assaying enzyme activity using sensors of the invention. Thus, one general class of embodiments provides methods of assaying an activity of an enzyme. In the methods, the enzyme is contacted with a sensor. The sensor includes 1) a substrate module comprising a substrate for the enzyme, wherein the substrate is in a first state on which the enzyme can act, thereby converting the substrate to a second state, and an environmentally sensitive label, and 2) a detection module, which detection module binds to the substrate module when the substrate is in the first state or the second state. Binding of the detection module to the substrate module results in a change in signal from the label. The change in signal from the label is detected and correlated to the activity of the enzyme, whereby the activity of the enzyme is assayed.

The assay can be, e.g., qualitative or quantitative. As a few examples, the assay can simply indicate whether the activity is present (e.g., a signal change is detected) or absent (e.g., no signal change is detected), or it can indicate the activity is higher or lower than activity in a corresponding control sample (e.g., the signal change is greater or less than that in a control assay or sample, e.g., one that includes a known quantity of enzyme or premodified substrate or the like), or it can be used to determine a number of activity units of the enzyme (an activity unit is typically defined as the amount of enzyme which will catalyze the transformation of 1 micromole of the substrate per minute under standard conditions).

The methods can be used, e.g., for in vitro biochemical assays of enzyme activity using purified or partially purified enzyme, a cell lysate, or the like, or they can be used to detect enzyme activity inside cells and/or organisms. Thus, in one class of embodiments, contacting the enzyme and the sensor comprises introducing the substrate module into a cell, e.g., a cell including or potentially including the enzyme. Similarly, in some embodiments, contacting the enzyme and the sensor comprises introducing the detection module into the cell. In other embodiments, the detection module is endogenous to the cell. For example, the detection module can be expressed from the cell's genome, from a nucleic acid construct transiently or stably transfected into the cell, or the like. Thus, the methods optionally include introducing a vector encoding the detection module into the cell, whereby the detection module is expressed in the cell.

Similarly, the enzyme can be endogenous to the cell or expressed from a nucleic acid construct transiently or stably transfected into the cell. In one class of embodiments, a vector encoding the enzyme is introduced into the cell, whereby the enzyme is expressed (e.g., overexpressed) in the cell. For example, such expression can result in the enzyme being present in the cell at an amount that is at least 2×, at least 5×, at least 10×, at least 50×, or even at least 100× normal for that cell type (including expression in a cell not normally expressing the enzyme).

The sensor is optionally introduced into a subcellular compartment, e.g., any of various organelles such as the nucleus, mitochondrion, chloroplast, lysosome, ER, Golgi, or the like.

The substrate module, detection module, and/or vector(s) encoding the detection module and/or the enzyme can be introduced into the cell simultaneously or sequentially, as desired. As just one example, a vector encoding the enzyme and the detection module can be introduced into the cell, the cell can be permitted to express the enzyme and detection module, and then the substrate module can be introduced into the cell. A variety of suitable techniques for introducing molecules into cells (e.g., lipofection, cyclodextran-mediated delivery, or association with a cellular delivery module) are described herein and/or are well known in the art.

In a preferred aspect, the environmentally sensitive label is a fluorescent label. The change in signal from the label can thus be a change in fluorescence emission intensity, fluorescence emission wavelength, and/or fluorescence duration. As noted previously, further details can be found, e.g., in the section entitled “Environmentally sensitive and fluorescent labels” below.

As noted previously, caging the sensor can permit initiation of the activity assay to be precisely controlled, temporally and/or spatially (see, e.g., US patent application publication 2004/0166553). Thus, in one class of embodiments, the sensor comprises one or more caging groups associated with the substrate module, which caging groups inhibit (e.g., prevent) the enzyme from acting upon the substrate. The methods include uncaging the substrate module, e.g., by exposing the substrate module to uncaging energy, thereby freeing the substrate module from inhibition by the one or more caging groups. Typically, the one or more caging groups prevent the enzyme from acting upon the substrate, and removal of or an induced conformational change in the one or more caging groups permits the enzyme to act upon the substrate.

The substrate module can be uncaged, for example, by exposing the substrate module to light of a first wavelength (for photoactivatable or photolabile caging groups), sonicating the substrate module, or otherwise supplying uncaging energy appropriate for the specific caging groups utilized.

Alternatively or in addition, the methods can include uncaging other caged reagents, for example, caged nucleotides (e.g., caged ATP, e.g., to initiate a kinase reaction), caged metal ions, caged chelating agents (e.g., caged EDTA or EGTA, e.g., to terminate a reaction requiring divalent cations), caged activators or inhibitors, or the like.

The methods can include contacting the enzyme with a modulator (e.g., an activator or inhibitor) of its activity. Similarly, the methods can include modulating the activity of at least one other enzyme, e.g., by adding an activator or inhibitor of at least one other enzyme that functions (or potentially functions) in an upstream, downstream, or related signaling or metabolic pathway.

In one aspect, the methods can be used to screen for compounds that affect activity of the enzyme (or binding of the substrate and detection modules to each other). Thus, in one class of embodiments, the methods include contacting the enzyme with a test compound, assaying the activity of the enzyme in the presence of the test compound, and comparing the activity of the enzyme in the presence of the test compound with the activity of the enzyme in the absence of the test compound. Screening methods are described in greater detail below, in the section entitled “Screening for modulators of enzyme activity.”

The methods can be used to monitor the activities of two or more enzymes, e.g., in a single reaction mixture. For example, if desired, a second sensor comprising a second substrate module including a second substrate for a second enzyme and a second environmentally sensitive label, whose signal is detectably different from that of the first sensor's label upon binding to a second detection module, is contacted with the second enzyme. The second detection module can be the same as or different from the first detection module. A signal change from the second label is detected and correlated with the activity of the second enzyme. As another example, the second sensor can comprise a polypeptide including an environmentally sensitive or fluorescent label (such as the polypeptides described above in the section entitled “Sensors including environmentally sensitive or fluorescent labels”).

Essentially all of the features noted for the compositions above apply to these methods as well, as relevant: for example, with respect to type of enzyme, exemplary substrate and detection modules, fluorescent labels, type of caging groups, use of cellular delivery modules, and/or the like.

Specificity of the assay can be adjusted in a number of ways, e.g., through choice of substrate, assay format, reaction conditions, and/or the like. For example, the substrate can be a specific substrate, acted on by only a single enzyme (e.g., under a defined set of reaction conditions), or it can be a generic substrate, acted on by two or more closely related enzymes or even by a large number of enzymes. A variety of detection modules can be used, e.g., from domains or antibodies that recognize only the modified form of a particular substrate to domains or antibodies that bind any of a family of related modified substrates. The particular enzyme of interest can be overexpressed in a cell, thus decreasing any background signal from other enzymes in the cell in a cell-based assay; this technique may be particularly useful, for example, in screening for activators or inhibitors of the enzyme.

Another general class of embodiments also provides methods of assaying an activity of an enzyme (e.g., a tyrosine kinase or tyrosine-specific phosphatase). In the methods, the enzyme is contacted with a sensor, whereby the enzyme optionally phosphorylates or dephosphorylates the sensor. The sensor includes an environmentally sensitive or fluorescent label whose signal changes upon phosphorylation or dephosphorylation of the sensor. The change in signal from the label is detected and correlated to the activity of the enzyme, whereby the activity of the enzyme is assayed.

In one class of embodiments, the sensor includes a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label, X⁻¹ and X⁺³ are independently selected from the group consisting of: A, V, I, L, M, F, Y, W, and an amino acid residue comprising the environmentally sensitive or fluorescent label, X⁺¹, X⁺², X⁺⁴, and X⁺⁵ are independently selected from the group consisting of: an amino acid residue and an amino acid residue comprising the environmentally sensitive or fluorescent label, and at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label. Phosphorylation or dephosphorylation of Y⁰ results in a change in signal from the label.

In another class of embodiments, the sensor includes a polypeptide comprising an environmentally sensitive or fluorescent label, which polypeptide comprises a tyrosine residue. When the tyrosine is unphosphorylated, it engages in an interaction with the label, and this interaction is at least partially disrupted when the tyrosine is phosphorylated, whereby a signal from the label changes upon phosphorylation or dephosphorylation of the tyrosine.

In yet another class of embodiments, the sensor includes a polypeptide substrate for a protein tyrosine kinase, which polypeptide substrate comprises an environmentally sensitive or fluorescent label. The environmentally sensitive or fluorescent label is located at amino acid position −2 or +3 with respect to the phosphorylation site within the polypeptide substrate, and phosphorylation or dephosphorylation of the substrate at the phosphorylation site results in a change in signal from the label.

As for the embodiments described above, the assay can be, e.g., qualitative or quantitative. As a few examples, the assay can simply indicate whether the activity is present (e.g., a signal change is detected) or absent (e.g., no signal change is detected), or it can indicate the activity is higher or lower than activity in a corresponding control sample (e.g., the signal change is greater or less than that in a control assay or sample, e.g., one that includes a known quantity of enzyme or premodified substrate or the like), or it can be used to determine a number of activity units of the enzyme.

The methods can be used, e.g., for in vitro biochemical assays of enzyme activity using purified or partially purified enzyme, a cell lysate, or the like, or they can be used to detect enzyme activity inside cells and/or organisms. Thus, in one class of embodiments, contacting the enzyme and the sensor comprises introducing the sensor into a cell, e.g., a cell including or potentially including the enzyme. The enzyme can be endogenous to the cell or expressed from a nucleic acid construct transiently or stably transfected into the cell. In one class of embodiments, a vector encoding the enzyme is introduced into the cell, whereby the enzyme is expressed (e.g., overexpressed) in the cell. For example, such expression can result in the enzyme being present in the cell at an amount that is at least 2×, at least 5×, at least 10×, at least 50×, or even at least 100× normal for that cell type (including expression in a cell not normally expressing the enzyme).

A variety of suitable techniques for introducing molecules into cells (e.g., lipofection, cyclodextran-mediated delivery, or association with a cellular delivery module) are described herein and/or are well known in the art. Similarly, the sensor is optionally introduced into a subcellular compartment, e.g., any of various organelles such as the nucleus, mitochondrion, chloroplast, lysosome, ER, Golgi, or the like.

In a preferred aspect, the label is a fluorescent label. The change in signal from the label can thus be a change in fluorescence emission intensity, fluorescence emission wavelength, and/or fluorescence duration. As noted previously, further details can be found, e.g., in the section entitled “Environmentally sensitive and fluorescent labels” below.

As noted previously, caging the sensor can permit initiation of the activity assay to be precisely controlled, temporally and/or spatially. Thus, in one class of embodiments, the sensor comprises one or more caging groups associated with the polypeptide or polypeptide substrate, which caging groups inhibit (e.g., prevent) the enzyme from acting upon the polypeptide or polypeptide substrate. The methods include uncaging the polypeptide or polypeptide substrate, e.g., by exposing the caged sensor to uncaging energy, thereby freeing the polypeptide or polypeptide substrate from inhibition by the one or more caging groups. Typically, the one or more caging groups prevent the enzyme from acting upon the polypeptide or polypeptide substrate, and removal of or an induced conformational change in the one or more caging groups permits the enzyme to act upon the polypeptide or polypeptide substrate.

The caged polypeptide or polypeptide substrate can be uncaged, for example, by exposing the caged sensor to light of a first wavelength (for photoactivatable or photolabile caging groups), sonicating the caged sensor, or otherwise supplying uncaging energy appropriate for the specific caging groups utilized.

Alternatively or in addition, the methods can include uncaging other caged reagents, for example, caged nucleotides (e.g., caged ATP, e.g., to initiate a kinase reaction), caged metal ions, caged chelating agents (e.g., caged EDTA or EGTA, e.g., to terminate a reaction requiring divalent cations), caged activators or inhibitors, or the like.

The methods can include contacting the enzyme with a modulator (e.g., an activator or inhibitor) of its activity. Similarly, the methods can include modulating the activity of at least one other enzyme, e.g., by adding an activator or inhibitor of at least one other enzyme that functions (or potentially functions) in an upstream, downstream, or related signaling or metabolic pathway.

In one aspect, the methods can be used to screen for compounds that affect activity of the enzyme. Thus, in one class of embodiments, the methods include contacting the enzyme with a test compound, assaying the activity of the enzyme in the presence of the test compound, and comparing the activity of the enzyme in the presence of the test compound with the activity of the enzyme in the absence of the test compound. Screening methods are described in greater detail below, in the section entitled “Screening for modulators of enzyme activity.”

In embodiments in which the sensor includes a tyrosine residue that interacts or potentially interacts with the label, the methods optionally include monitoring the interaction or suspected interaction of the tyrosine with the label. For example, the methods optionally include performing NMR spectroscopy on an unphosphorylated form of the sensor to produce a first set of data and on a phosphorylated form of the sensor to produce a second set of data, and analyzing the first and second sets of data to determine whether the tyrosine residue interacts with the label when unphosphorylated and whether this interaction is at least partially disrupted when the tyrosine is phosphorylated.

Essentially all of the features noted for the compositions and methods above apply to these methods as well, as relevant: for example, with respect to type of enzyme, exemplary sensors, fluorescent labels, type of caging groups, use of cellular delivery modules, use of a second sensor, and/or the like.

### Screening for Modulators of Enzyme Activity

In one aspect, the invention provides methods of determining whether a test compound affects an activity of an enzyme. In the methods, a cell comprising the enzyme is provided, and a sensor is introduced into the cell.

In one class of embodiments, the sensor includes a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of D, E, and an amino acid residue comprising the environmentally sensitive or fluorescent label, X⁻¹ and X⁺³ are independently selected from the group consisting of: A, V, I, L, M, F, Y, W, and an amino acid residue comprising the environmentally sensitive or: fluorescent label, X⁺¹, X⁺², X⁺⁴, and X⁺⁵ are independently selected from the group consisting of: an amino acid residue and an amino acid residue comprising the environmentally sensitive or fluorescent label, and at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label. Phosphorylation or dephosphorylation of Y⁰ results in a change in signal from the label.

In another class of embodiments, the sensor includes a polypeptide comprising an environmentally sensitive or fluorescent label, which polypeptide comprises a tyrosine residue. When the tyrosine is unphosphorylated, it engages in an interaction with the label, and this interaction is at least partially disrupted when the tyrosine is phosphorylated, whereby a signal from the label changes upon phosphorylation or dephosphorylation of the tyrosine.

In yet another class of embodiments, the sensor includes a polypeptide substrate for a protein tyrosine kinase, which polypeptide substrate comprises an environmentally sensitive or fluorescent label. The environmentally sensitive or fluorescent label is located at amino acid position −2 or +3 with respect to the phosphorylation site within the polypeptide substrate, and phosphorylation or dephosphorylation of the substrate at the phosphorylation site results in a change in signal from the label.

In yet another class of embodiments, the sensor includes 1) a substrate module comprising a substrate for the enzyme, wherein the substrate is in a first state on which the enzyme can act, thereby converting the substrate to a second state, and an environmentally sensitive label, and 2) a detection module, which detection module binds to the substrate module when the substrate is in the first state or the second state, wherein binding of the detection module to the substrate module results in a change in signal from the label.

Regardless of which type of sensor is employed, the cell is contacted with the test compound, and the change in signal from the label is detected. The change provides an indication of the activity of the enzyme in the presence of the test compound. Typically, the activity of the enzyme in the presence of the test compound is compared to an activity of the enzyme in the absence of the test compound, to determine whether the test compound increases, decreases, or does not substantially affect the enzyme's activity.

As for the embodiments above, the enzyme can be endogenous to the cell or expressed from a nucleic acid construct transiently or stably transfected into the cell. In one class of embodiments, providing the cell comprising the enzyme comprises introducing a vector (e.g., an expression vector) encoding the enzyme into the cell, whereby the enzyme is expressed (e.g., overexpressed) in the cell. For example, such expression can result in the enzyme being present in the cell at an amount that is at least 2×, at least 5×, at least 10×, at least 50×, or even at least 100× normal for that cell type (including expression in a cell not normally expressing the enzyme).

Overexpression of the enzyme can, e.g., increase the sensitivity of the methods by helping ensure that activity of the desired enzyme is being monitored by the sensor (e.g., that modification of the substrate is due to the overexpressed enzyme instead of, or to a much greater extent than, to the action of one or more enzymes endogenous to the cell). Similarly, overexpression of the enzyme can, e.g., enable use of a less specific substrate (e.g., a generic or universal substrate rather than a specific substrate, e.g., a substrate that can be acted upon by a group of related enzymes (e.g., Src family kinases or kinases related by sequence homology to PKC)) in the sensor, since most modification of the substrate will be due to the overexpressed enzyme rather than to any endogenous enzymes which happen to act on the substrate.

In embodiments in which the sensor includes a substrate module and a detection module, introducing the sensor into the cell optionally comprises introducing the substrate module and the detection module into the cell. In another exemplary class of embodiments, introducing the sensor into the cell comprises introducing the substrate module and a vector encoding the detection module into the cell, whereby the detection module is expressed in the cell. The substrate module, detection module, and/or vector(s) encoding the detection module and/or the enzyme can be introduced into the cell simultaneously or sequentially, as desired. As just one example, a vector encoding the enzyme and the detection module can be introduced into the cell, the cell can be permitted to express the enzyme and detection module, and then the substrate module can be introduced into the cell. A variety of suitable techniques for introducing molecules into cells (e.g., lipofection, cyclodextran-mediated delivery, or association with a cellular delivery module) are described herein and/or are well known in the art.

Essentially all of the features noted for the compositions and methods above apply to these methods as well, as relevant: for example, with respect to type of enzyme (e.g., kinase or phosphatase), exemplary sensors, exemplary substrate and detection modules, fluorescent labels, use of caging groups, use of cellular delivery modules, and/or the like.

The methods of the invention offer a number of advantages as compared to traditional methods of screening for potential modulators and assaying enzyme activity. For example, overexpressing the enzyme in the cell can help ensure that activity of the desired enzyme is being monitored. As another example, when screening for modulators, a simple counterscreen can ensure that the modulator is affecting the desired step. (For example, in an exemplary kinase assay in which the detection module binds to a phosphorylated substrate, if treatment with a test compound decreases or eliminates the signal change observed when the sensor is phosphorylated in an untreated cell, a phosphorylated version of the substrate module can be prepared and introduced into a cell contacted with the test compound. If the compound inhibits kinase activity, a signal change from the pre-phosphorylated sensor should be observed, while if the compound interferes with a downstream step, e.g., interaction of the substrate and detection modules, the signal change would not be observed.) Another advantage, e.g., for kinase assays, is that the assay can be performed in the presence of either high or low concentrations of ATP to determine whether a particular test compound that inhibits kinase activity does so competitively or noncompetitively with respect to ATP.

### Kits

Kits comprising components of compositions of the invention and/or that can be used in practicing the methods of the invention form another feature of the invention. In one class of embodiments, the kit includes a sensor for detecting an activity of an enzyme, packaged in one or more containers. The sensor includes 1) a substrate module comprising a substrate for the enzyme, wherein the substrate is in a first state on which the enzyme can act, thereby converting the substrate to a second state, and an environmentally sensitive label, and 2) a detection module, which detection module binds to the substrate module when the substrate is in the first state, or which detection module binds to the substrate module when the substrate is in the second state, wherein binding of the detection module to the substrate module results in a change in signal from the label. Typically, the kit also includes instructions for using the sensor to detect the activity of the enzyme. The kit optionally also includes one or more buffers, transfection reagents, controls including a known quantity of the enzyme, and/or the like.

In another class of embodiments, a kit includes a substrate module and a nucleic acid encoding a detection module, packaged in one or more containers. The substrate module comprises a substrate for an enzyme, wherein the substrate is in a first state on which the enzyme can act, thereby converting the substrate to a second state, and an environmentally sensitive label. The detection module binds to the substrate module when the substrate is in the first state or in the second state, and binding of the detection module to the substrate module results in a change in signal from the label. Typically, the kit also includes instructions for using the substrate and detection modules as a sensor to detect the activity of the enzyme. The kit optionally also includes one or more buffers, transfection reagents, controls including a known quantity of the enzyme, and/or the like.

In yet another class of embodiments, a kit includes a substrate module and a cell comprising a nucleic acid encoding an enzyme and/or a nucleic acid encoding a detection module, packaged in one or more containers. The substrate module comprises a substrate for the enzyme, wherein the substrate is in a first state on which the enzyme can act, thereby converting the substrate to a second state, and an environmentally sensitive label. The detection module binds to the substrate module when the substrate is in the first state or in the second state, and binding of the detection module to the substrate module results in a change in signal from the label. Typically, the kit also includes instructions for using the kit to detect the activity of the enzyme. The kit optionally also includes one or more buffers, transfection reagents, controls including a known quantity of the enzyme, the detection module or a nucleic acid encoding the detection module if it is not already present in the cell, and/or the like.

In yet another class of embodiments, a kit includes a sensor for detecting an activity of an enzyme, packaged in one or more containers. In one class of embodiments, the sensor includes a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵. X⁻⁴, X⁻³, and X⁻² are independently selected from the group consisting of: D, E, and an amino acid residue comprising the environmentally sensitive or fluorescent label, X⁻¹ and X⁺³ are independently selected from the group consisting of: A, V, I, L, M, F, Y, W, and an amino acid residue comprising the environmentally sensitive or fluorescent label, X⁺¹, X⁺², X⁺⁴, and X⁺⁵ are independently selected from the group consisting of: an amino acid residue and an amino acid residue comprising the environmentally sensitive or fluorescent label, and at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label. Phosphorylation or dephosphorylation of Y⁰ results in a change in signal from the label.

In another class of embodiments, the sensor includes a polypeptide comprising an environmentally sensitive or fluorescent label, which polypeptide comprises a tyrosine residue. When the tyrosine is unphosphorylated, it engages in an interaction with the label, and this interaction is at least partially disrupted when the tyrosine is phosphorylated, whereby a signal from the label changes upon phosphorylation or dephosphorylation of the tyrosine.

In yet another class of embodiments, the sensor includes a polypeptide substrate for a protein tyrosine kinase, which polypeptide substrate comprises an environmentally sensitive or fluorescent label. The environmentally sensitive or fluorescent label is located at amino acid position −2 or +3 with respect to the phosphorylation site within the polypeptide substrate, and phosphorylation or dephosphorylation of the substrate at the phosphorylation site results in a change in signal from the label.

Typically, the kit also includes instructions for using the sensor to detect the activity of the enzyme. The kit optionally also includes one or more buffers, transfection reagents, controls including a known quantity of the enzyme, and/or the like. The kit optionally also includes a cell comprising a nucleic acid encoding the enzyme.

### Systems

In one aspect, the invention includes systems, e.g., systems used to practice the methods herein and/or comprising the compositions described herein. The system can include, e.g., a fluid handling element, a fluid containing element, a laser for exciting a fluorescent label, a detector for detecting a signal from a label (e.g., fluorescent emissions from a fluorescent label), a source of uncaging energy for uncaging caged sensors, and/or a robotic element that moves other components of the system from place to place as needed (e.g., a multiwell plate handling element). For example, in one class of embodiments, a composition of the invention is contained in a microplate reader or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element, and/or laser). The computer can also receive data from other components of the system, e.g., from a detector, and can interpret the data (e.g., by correlating a change in signal from the label with an activity of an enzyme), provide it to a user in a human readable format, or use that data to initiate further operations, in accordance with any programming by the user.

### Environmentally Sensitive and Fluorescent Labels

As noted, a sensor of this invention optionally includes an environmentally sensitive label, e.g., an environmentally sensitive fluorescent, luminescent, solvatochromatic, or magnetic label. In a preferred aspect, the environmentally sensitive label attached to a substrate module, polypeptide, or polypeptide substrate of the invention is a fluorescent label. The signal from an environmentally sensitive label changes when the environment of the label changes. For example, the fluorescence of an environmentally sensitive fluorescent label changes when the hydrophobicity, pH, and/or the like of the label's environment changes (e.g., upon binding of the substrate module with which the label is associated to a detection module, such that the label is transferred from an aqueous environment to a more hydrophobic environment at the binding interface between the modules). Typically, the signal from an environmentally sensitive label is affected by the solvent in which the label is located. For example, the signal from an environmentally sensitive fluorescent label is typically significantly different when the label is in an aqueous solution versus in a less polar solvent (e.g., methanol) versus in a nonpolar solvent (e.g., hexane).

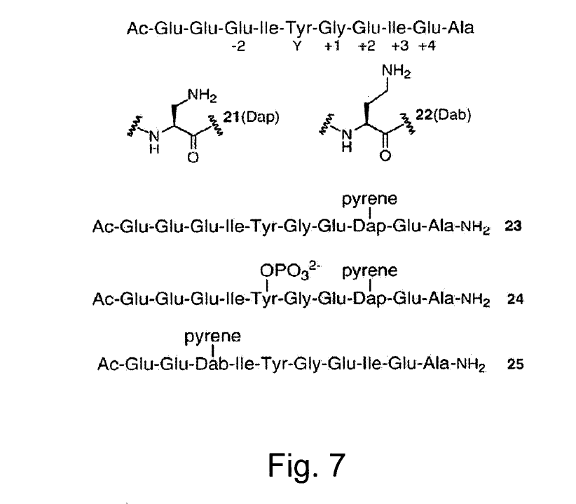

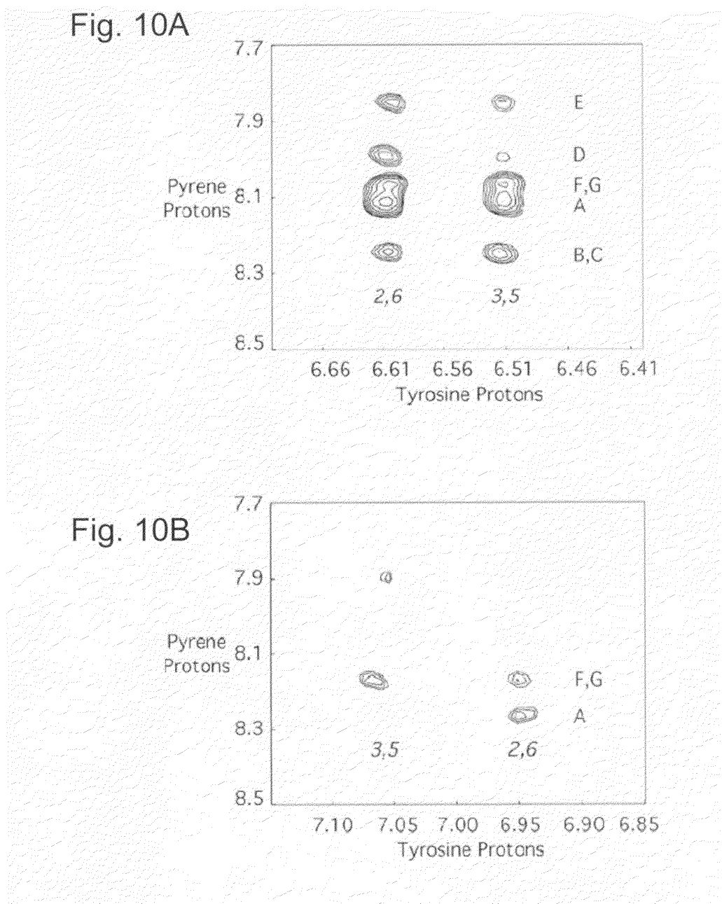

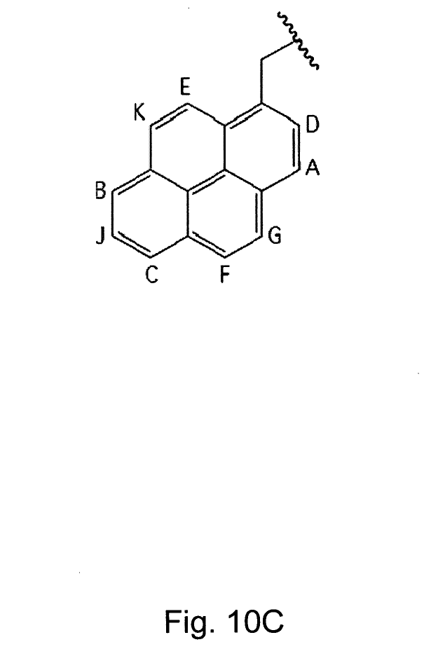

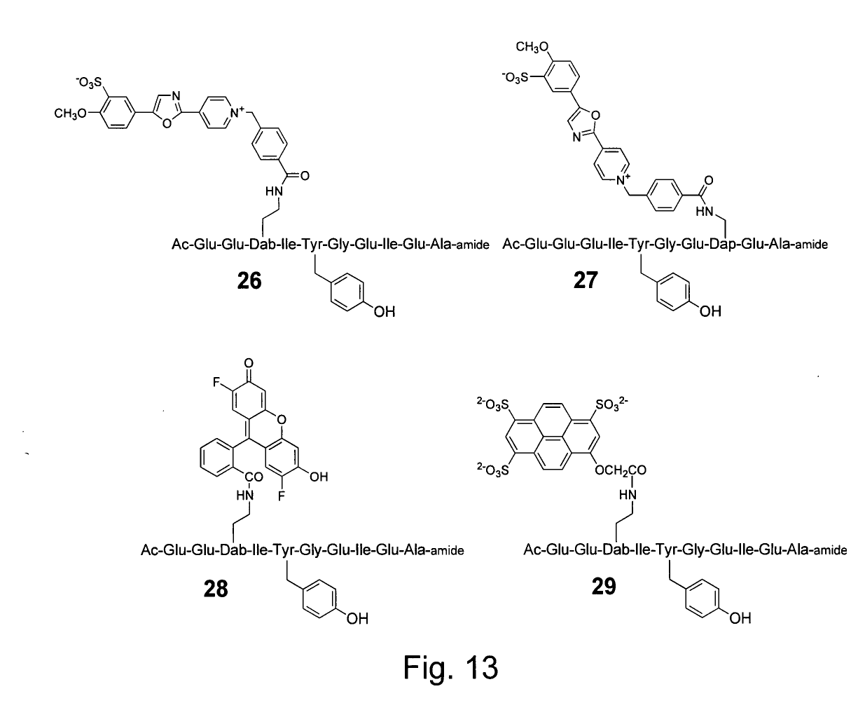

A number of environmentally sensitive fluorescent labels, many of which are commercially available, have been described in the art and can be adapted to the practice of the present invention. Examples of environmentally sensitive fluorophores include, but are not limited to, dapoxyl, NBD, Cascade Yellow, dansyl, PyMPO, pyrene, 7-diethylaminocoumarin-3-carboxylic acid, Marina Blue™, Pacific Blue™, Cascade Blue™, 2-anthracenesulfonyl, PyMPO, and 3,4,9,10-perylene-tetracarboxylic acid, and derivatives thereof (see, e.g., FIG. 25-7, FIG. 10 Panel C and FIG. 13). Reactive forms of these fluorophores are commercially available e.g., from Molecular Probes, Inc., or can readily be prepared by one of skill in the art. Other environmentally sensitive fluorescent labels have been described in, e.g., US patent application publication 20020055133 by Hahn et al. entitled “Labeled peptides, proteins and antibodies and processes and intermediates useful for their preparation”; Vazquez et al. (2004) “A new environment-sensitive fluorescent amino acid for Fmoc-based solid phase peptide synthesis” Org. Biomol. Chem. 2:1965-1966; Vazquez et al. (2003) “Fluorescent caged phosphoserine peptides as probes to investigate phosphorylation-dependent protein associations” J. Am. Chem. Soc. 125:10150-10151; Vazquez et al. (2005) “Photophysics and biological applications of the environment-sensitive fluorophore 6-N,N-dimethylamino-2,3-naphthalimide” J. Am. Chem. Soc. 127:1300-1306; and Cousins-Wasti et al. (1996) “Determination of affinities for lck SH2 binding peptides using a sensitive fluorescence assay: Comparison between the pYEEIP and pYQPQP consensus sequences reveals context-dependent binding specificity” Biochemistry 35:16746-16752.

Fluorescent labels are not all environmentally sensitive, and as indicated above, environmentally insensitive labels can be employed in certain embodiments. The fluorescence of an environmentally insensitive fluorescent label is typically not significantly affected by the solvent in which the label is located. For example, the signal from an environmentally insensitive fluorescent label is typically not significantly different whether the label is in an aqueous solution, a less polar solvent (e.g., methanol), or a nonpolar solvent (e.g., hexane). Examples of environmentally insensitive fluorophores include, but are not limited to, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethylrhodamine (6-TAMRA), and BODIPY FL, and derivatives thereof. Reactive forms of these fluorophores are commercially available e.g., from Molecular Probes, Inc., or can readily be prepared by one of skill in the art and used for incorporation of the labels into desired molecules. A variety of additional fluorescent labels are known in the art, including, e.g., bimane and Alexa Fluor 350, 405, 488, 500, 514, 532, 546, 555, 568, 594, 610, 633, 647, 660, 680, 700, and 750, among many others. Fluorescent labels employed in the invention are optionally small molecules, e.g., having a molecular weight of less than about 1000 daltons.

Signals from the environmentally sensitive and/or fluorescent labels can be detected by essentially any method known in the art (e.g., fluorescence spectroscopy, fluorescence microscopy, etc.). Excitation and emission wavelengths for the exemplary fluorophores described above can be found, e.g., in Haughland (2003) Handbook of Fluorescent Probes and Research Products Ninth Edition, available from Molecular Probes (or on the world wide web at probes.com/handbook), or in The Handbook—A Guide to Fluorescent Probes and Labeling Technologies, Tenth Edition, available on the internet at probes.invitrogen.com/handbook, and in the references above.

The change in signal from a fluorescent label (e.g., an environmentally sensitive or an environmentally insensitive fluorescent label) can be, e.g., a change in fluorescence emission intensity, fluorescence emission wavelength, and/or fluorescence duration. The change in signal from the label is optionally a change of greater than ±25%, greater than ±50%, greater than ±75%, greater than ±90%, greater than ±95%, greater than ±98%, greater than +100%, greater than +200%, greater than +300%, greater than +400%, greater than +500%, greater than +600%, or greater than +700% in fluorescence emission intensity.

Labels can be attached to molecules (e.g., substrates) during synthesis or by postsynthetic reactions by techniques established in the art. For example, a fluorescently labeled nucleotide can be incorporated into a nucleic acid during enzymatic or chemical synthesis of the nucleic acid, e.g., at a preselected or random nucleotide position. Alternatively, fluorescent labels can be added to nucleic acids by postsynthetic reactions, at either random or preselected positions (e.g., an oligonucleotide can be chemically synthesized with a terminal amine or free thiol at a preselected position, and a fluorophore can be coupled to the oligonucleotide via reaction with the amine or thiol). As another example, a fluorescently labeled residue can be incorporated into a polypeptide during enzymatic or chemical synthesis of the polypeptide. Alternatively, fluorescent labels can be added to polypeptides by postsynthetic reactions. A polypeptide substrate optionally comprises one or more residues incorporated to facilitate attachment of the label, e.g., an (L)-2,3-diaminopropionic acid (Dap), (L)-2,4-diaminobutyric acid (Dab), ornithine, lysine, cysteine, or homocysteine residue (or essentially any other chemically reactive natural or unnatural amino acid derivative or residue) to which the environmentally sensitive label is attached. See, e.g., Examples 1 and 3 herein, and Hahn et al., Vazquez et al. (2004), Vazquez et al. (2003), Vazquez et al. (2005), and Cousins-Wasti et al. (1996), all supra.

Substrate and/or detection modules of the invention optionally include a second, non-environmentally sensitive label, e.g., a fluorophore or quantum dot, whose signal is not dependent on binding of the substrate and detection modules. Similarly, polypeptides or polypeptide substrates of the invention including an environmentally sensitive or fluorescent label optionally also include a second label that is not environmentally sensitive and/or whose signal is not dependent on the phosphorylation state of the polypeptide or polypeptide substrate. Such second labels can be used, e.g., for monitoring transfection efficiency (e.g., normalizing for differences in delivery of the sensors into cells), correcting for well-to-well or day-to-day deviation, and the like.

### In Vivo and In Vitro Cellular Delivery

Molecules (e.g., the substrate and/or delivery modules of enzyme sensors or the labeled polypeptides or polypeptide substrates) can be introduced into cells by traditional methods such as lipofection, electroporation, microinjection, optofection, laser transfection, calcium phosphate precipitation, cyclodextran-mediated delivery, and/or particle bombardment. Alternatively, the molecule (e.g., the substrate and/or delivery module, polypeptide, or polypeptide substrate) can be associated (covalently or non-covalently) with a cellular delivery module that can mediate its introduction into the cell. The cellular delivery module is typically, but need not be, a polypeptide, for example, a PEP-1 peptide, an amphipathic peptide, e.g., an MPG peptide (Simeoni et al. (2003) “Insight into the mechanism of the peptide-based gene delivery system MPG: Implications for delivery of siRNA into mammalian cells” Nucl Acids Res 31: 2717-2724), a cationic peptide (e.g., a homopolymer of lysine, histidine, or D-arginine), or a protein transduction domain (a polypeptide that can mediate introduction of a covalently associated molecule into a cell). See, e.g., Lane (2001) Bioconju Chem., 12:825-841; Bonetta (2002) The Scientist 16:38; and Schwartz and Zhang (2000) Curr Opin Mol Ther 2:162-7. For example, a molecule can be covalently associated with a protein transduction domain (e.g., a protein transduction domain derived from an HIV-1 Tat protein, from a herpes simplex virus VP22 protein, or from a Drosophila antennapedia protein, or a model protein transduction domain, e.g., a short D-arginine homopolymer, e.g., 8-D-Arg, eight contiguous D-arginine residues). The protein transduction domain-coupled molecule can simply be, e.g., added to cell culture or injected into an animal for delivery. (Note that TAT and D-arginine homopolymers, for example, can alternatively be noncovalently associated with the molecule and still mediate its introduction into the cell.)

A number of polypeptides capable of mediating introduction of associated molecules into a cell are known in the art and can be adapted to the present invention; see, e.g., the references above and Langel (2002) Cell Penetrating Peptides CRC Press, Pharmacology & Toxicology Series.

Molecules can also be introduced into cells by covalently or noncovalently attached lipids, e.g., by lipofection or by a covalently attached myristoyl group.

In summary, substrate and/or delivery modules, polypeptides, and polypeptide substrates can be introduced into a cell by any of several methods, including without limitation, lipofection, cyclodextran, electroporation, microinjection, and covalent or noncovalent association with a cellular delivery module. They can optionally be introduced into specific tissues and/or cell types (e.g., explanted or in an organism), for example, by laser transfection, gold particle bombardment, microinjection, coupling to viral proteins, or covalent association with a protein transduction domain, among other techniques. See, e.g., Robbins et al. (2002) “Peptide delivery to tissues via reversibly linked protein transduction sequences” Biotechniques 33:190-192 and Rehman et al. (2003) “Protection of islets by in situ peptide-mediated transduction of the I-kappa B kinase inhibitor Nemo-binding domain peptide” J Biol Chem 278:9862-9868.

The cell into which a substrate and/or delivery module, polypeptide, or polypeptide substrate of this invention is introduced can be a prokaryotic cell (e.g., a bacterial cell) or a eukaryotic cell (e.g., a yeast, a vertebrate cell, a mammalian cell, a rodent cell, a primate cell, a human cell, a plant cell, an insect cell, or essentially any other type of eukaryotic cell). The cell can be, e.g., in culture or in a tissue, fluid, etc. and/or from or in an organism.

If the molecule is caged, such delivery can be accomplished without uncaging and thereby activating the molecule; for example, a photoactivatable substrate module, polypeptide, or polypeptide substrate is not available for enzymatic modification during the delivery process until exposed to light of appropriate wavelength.

The cellular delivery modules are optionally caged. Covalently associated cellular delivery modules (e.g., protein transduction domains) can optionally be released from the associated molecule, e.g., by placement of a photolabile linkage, a disulfide or ester linkage that is reduced or cleaved in the cell, or the like, between the cellular delivery module and the molecule. For example, an 8-D-Arg module can be covalently linked through a disulfide linker to a substrate module, polypeptide, or polypeptide substrate. The 8-D-Arg module mediates entry of the substrate module, polypeptide, or polypeptide substrate into a cell, where the linker is reduced in the reducing environment of the cytoplasm, freeing the substrate module, polypeptide, or polypeptide substrate from the 8-D-Arg module.

The amount of a substrate and/or delivery module, polypeptide, or polypeptide substrate delivered to a cell can optionally be controlled by controlling the number of cellular delivery modules associated with the substrate and/or delivery module, polypeptide, or polypeptide substrate (covalently or noncovalently). For example, increasing the ratio of 8-D-Arg to substrate module, polypeptide, or polypeptide substrate can increase the percentage of substrate module, polypeptide, or polypeptide substrate that enters the cell.

The substrate and/or delivery modules, polypeptides, and polypeptide substrates of this invention optionally also comprise a subcellular delivery module (e.g., a peptide, nucleic acid, and/or carbohydrate tag) or other means of achieving a desired subcellular localization (e.g., at which the enzyme is or is suspected to be present). Examples of subcellular delivery modules include nuclear localization signals, chloroplast stromal targeting sequences, and many others (see, e.g., Molecular Biology of the Cell (3rd ed.) Alberts et al., Garland Publishing, 1994; and Molecular Cell Biology (4th ed.) Lodish et al., W H Freeman & Co, 1999). Similarly, localization can be to a target protein; that is, the subcellular delivery module can comprise a binding domain that binds the target protein.

### Caging Groups

A large number of caging groups, and a number of reactive compounds that can be used to covalently attach caging groups to other molecules, are well known in the art. Examples of photolabile caging groups include, but are not limited to: nitroindolines; N-acyl-7-nitroindolines; phenacyls; hydroxyphenacyl; brominated 7-hydroxycoumarin-4-ylmethyls (e.g., Bhc); benzoin esters; dimethoxybenzoin; meta-phenols; 2-nitrobenzyl; 1-(4,5-dimethoxy-2-nitrophenyl)ethyl (DMNPE); 4,5-dimethoxy-2-nitrobenzyl (DMNB); alpha-carboxy-2-nitrobenzyl (CNB); 1-(2-nitrophenyl)ethyl (NPE); 5-carboxymethoxy-2-nitrobenzyl (CMNB); (5-carboxymethoxy-2-nitrobenzyl)oxy) carbonyl; (4,5-dimethoxy-2-nitrobenzyl)oxy) carbonyl; desoxybenzoinyl; and the like. See, e.g., U.S. Pat. No. 5,635,608 to Haugland and Gee (Jun. 3, 1997) entitled “α-carboxy caged compounds”; Neuro 19, 465 (1997); J Physiol 508.3, 801 (1998); Proc Natl Acad Sci USA 1988 Sep, 85(17):6571-5; J Biol Chem 1997 Feb. 14, 272(7):4172-8; Neuron 20, 619-624, 1998; Nature Genetics, vol. 28:2001:317-325; Nature, vol. 392, 1998:936-941; Pan, P., and Bayley, H. “Caged cysteine and thiophosphoryl peptides” FEBS Letters 405:81-85 (1997); Pettit et al. (1997) “Chemical two-photon uncaging: a novel approach to mapping glutamate receptors” Neuron 19:465-471; Furuta et al. (1999) “Brominated 7-hydroxycoumarin-4-ylmethyls: novel photolabile protecting groups with biologically useful cross-sections for two photon photolysis” Proc. Natl. Acad. Sci. 96(4):1193-1200; Zou et al. “Catalytic subunit of protein kinase A caged at the activating phosphothreonine” J. Amer. Chem. Soc. (2002) 124:8220-8229; Zou et al. “Caged Thiophosphotyrosine Peptides” Angew. Chem. Int. Ed. (2001) 40:3049-3051; Conrad I I et al. “p-Hydroxyphenacyl Phototriggers: The reactive Excited State of Phosphate Photorelease” J. Am. Chem. Soc. (2000) 122:9346-9347; Conrad I I et al. “New Phototriggers 10: Extending the π,π* Absorption to Release Peptides in Biological Media” Org. Lett. (2000) 2:1545-1547; Givens et al. “A New Phototriggers 9: p-Hydroxyphenacyl as a C-Terminus Photoremovable Protecting Group for Oligopeptides” J. Am. Chem. Soc. (2000) 122:2687-2697; Bishop et al. “40-Aminomethyl-2,20-bipyridyl-4-carboxylic Acid (Abc) and Related Derivatives: Novel Bipyridine Amino Acids for the Solid-Phase Incorporation of a Metal Coordination Site Within a Peptide Backbone” Tetrahedron (2000) 56:4629-4638; Ching et al. “Polymers As Surface-Based Tethers with Photolytic triggers Enabling Laser-Induced Release/Desorption of Covalently Bound Molecules” Bioconjugate Chemistry (1996) 7:525-8; BioProbes Handbook, 2002 from Molecular Probes, Inc.; and Handbook of Fluorescent Probes and Research Products, Ninth Edition or Web Edition, from Molecular Probes, Inc, as well as the references below. Many compounds, kits, etc. for use in caging various molecules are commercially available, e.g., from Molecular Probes, Inc. (on the world wide web at molecularprobes.com).

Environmentally responsive polymers suitable for use as caging groups have also been described. Such polymers undergo conformational changes induced by light, an electric or magnetic field, a change in pH and/or ionic strength, temperature, or addition of an antigen or saccharide, or other environmental variables. For example, Shimoboji et al. (2002) “Photoresponsive polymer-enzyme switches” Proc. Natl. Acad. Sci. USA 99:16,592-16,596 describes polymers that undergo reversible conformational changes in response to light. Such polymers can, e.g., be used as photoactivatable caging groups. See US patent application publication 2004/0166553. See also Ding et al. (2001) “Size-dependent control of the binding of biotinylated proteins to streptavidin using a polymer shield” Nature 411:59-62; Miyata et al. (1999) “A reversibly antigen-responsive hydrogel” Nature 399:766-769; Murthy et al. (2003) “Bioinspired pH-responsive polymers for the intracellular delivery of biomolecular drugs” Bioconjugate Chem. 14:412-419; and Galaev and Mattiasson (1999) “‘Smart’ polymers and what they could do in biotechnology and medicine” Trends Biotech. 17:335-340.

An alternative method for caging a molecule is to enclose the molecule in a photolabile vesicle (e.g., a photolabile lipid vesicle), optionally including a protein transduction domain or the like. Similarly, the molecule can be loaded into the pores of a porous bead which is then encased in a photolabile gel. As another alternative, a caging group optionally comprises a first binding moiety that can bind to a second binding moiety. For example, the caging group can include a biotin (the first binding moiety in this example); a second binding moiety, e.g., streptavidin or avidin, can thus be bound to the caging group, increasing its bulkiness and its effectiveness at caging. In certain embodiments, a caged component comprises two or more caging groups each comprising a first binding moiety, and the second binding moiety can bind two or more first binding moieties simultaneously. See US patent application publication 2004/0166553.

Caged polypeptides (including, e.g., peptide substrates, substrate modules, and detection modules) can be produced, e.g., by reacting a polypeptide with a caging compound or by incorporating a caged amino acid during synthesis of a polypeptide. See, e.g., Tatsu et al. (1996) “Solid-phase synthesis of caged peptides using tyrosine modified with a photocleavable protecting group: Application to the synthesis of caged neuropeptide Y” Biochem Biophys Res Comm 227:688-693, which describes synthesis of polypeptides including tyrosine residues caged with 2-nitrobenzyl groups; Veldhuyzen et al. (2003) “A light-activated probe of intracellular protein kinase activity” J Am Chem Soc 125:13358-9, which describes synthesis of a polypeptide including a caged serine; and Vazquez et al. (2003) “Fluorescent caged phosphoserine peptides as probes to investigate phosphorylation-dependent protein associations” J. Am. Chem. Soc. 125:10150-10151, which describes synthesis of a polypeptide including a caged phosphoserine. See also, e.g., U.S. Pat. No. 5,998,580 to Fay et al. (Dec. 7, 1999) entitled “Photosensitive caged macromolecules”; Kossel et al. (2001) PNAS 98:14702-14707; Trends Plant Sci (1999) 4:330-334; PNAS (1998) 95:1568-1573; J Am Chem Soc (2002) 124:8220-8229; Pharmacology & Therapeutics (2001) 91:85-92; and Angew Chem Int Ed Engl (2001) 40:3049-3051. A photolabile polypeptide linker (e.g., for connecting a protein transduction domain and a sensor, or the like) can, for example, comprise a photolabile amino acid such as that described in U.S. Pat. No. 5,998,580, supra.

Caged nucleic acids (e.g., DNA, RNA or PNA) can be produced by reacting the nucleic acids with caging compounds or by incorporating a caged nucleotide during synthesis of a nucleic acid. See, e.g., U.S. Pat. No. 6,242,258 to Haselton and Alexander (Jun. 5, 2001) entitled “Methods for the selective regulation of DNA and RNA transcription and translation by photoactivation”; Nature Genetics (2001) 28: 317-325; and Nucleic Acids Res. (1998) 26:3173-3178.

Caged modulators (e.g., inhibitors and activators), small molecules, etc. can be similarly produced by reaction with caging compounds or by synthesis. See, e.g., Trends Plant Sci (1999) 4:330-334; PNAS (1998) 95:1568-1573; U.S. Pat. No. 5,888,829 to Gee and Millard (Mar. 30, 1999) entitled “Photolabile caged ionophores and method of using in a membrane separation process”; U.S. Pat. No. 6,043,065 to Kao et al. (Mar. 28, 2000) entitled “Photosensitive organic compounds that release 2,5,-di(tert-butyl)hydroquinone upon illumination”; U.S. Pat. No. 5,430,175 to Hess et al. (Jul. 4, 1995) entitled “Caged carboxyl compounds and use thereof”; U.S. Pat. No. 5,872,243; and PNAS (1980) 77:7237-41. A number of caged compounds, including for example caged nucleotides, caged Ca2+, caged chelating agents, caged neurotransmitters, and caged luciferin, are commercially available, e.g., from Molecular Probes, Inc. (on the world wide web at molecularprobes.com).

Useful site(s) of attachment of caging groups to a given molecule can be determined by techniques known in the art. For example, a molecule with a known activity can be reacted with a caging compound. The resulting caged molecule can then be tested to determine if its activity is sufficiently abrogated. As another example, amino acid residues central to the activity of a polypeptide substrate (e.g., a residue modified by the enzyme, residues located at a binding interface, or the like) can be identified by routine techniques such as scanning mutagenesis, sequence comparisons and site-directed mutagenesis, or the like. Such residues can then be caged, and the activity of the caged substrate can be assayed to determine the efficacy of caging.

Appropriate methods for uncaging caged molecules are also known in the art. For example, appropriate wavelengths of light for removing many photolabile groups have been described; e.g., 300-360 nm for 2-nitrobenzyl, 350 nm for benzoin esters, and 740 nm for brominated 7-hydroxycoumarin-4-ylmethyls (two-photon) (see, e.g., references herein). Conditions for uncaging any caged molecule (e.g., the optimal wavelength for removing a photolabile caging group) can be determined according to methods well known in the art. Instrumentation and devices for delivering uncaging energy are likewise known (e.g., sonicators, heat sources, light sources, and the like). For example, well-known and useful light sources include e.g., a lamp, a laser (e.g., a laser optically coupled to a fiber-optic delivery system) or a light-emitting compound. See also U.S. patent application Ser. No. 10/716,176 by Witney et al. entitled “Uncaging devices.”

### Molecular Biological Techniques

In practicing the present invention, many conventional techniques in molecular biology, microbiology, and recombinant DNA technology are optionally used (e.g., for making and/or manipulating nucleic acids, polypeptides, and/or cells of the invention). These techniques are well known, and detailed protocols for numerous such procedures (including, e.g., in vitro amplification of nucleic acids, cloning, mutagenesis, transformation, cellular transduction with nucleic acids, protein expression, and/or the like) are described in, for example, Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2002 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2004) (“Ausubel”)). Other useful references, e.g. for cell isolation and culture include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla. A variety of vectors, including expression vectors, have been described and are readily available to one of skill, as are a large number of cells and cell lines suitable for the maintenance and use of such vectors.

### Polypeptide Production

Polypeptides (e.g., polypeptide substrates, detection modules, substrate modules, or cellular delivery modules) can optionally be produced by expression in a host cell transformed with a vector comprising a nucleic acid encoding the desired polypeptide(s). Expressed polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography, for example. Protein refolding steps can be used, as desired, in completing configuration of the mature protein. Finally, high performance liquid chromatography (HPLC) can be employed in the final purification steps. See, e.g., the references noted above and Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ; Harris and Angal (1990) Protein Purification Applications: A Practical Approach IRL Press at Oxford, Oxford, U.K.; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

Alternatively, cell-free transcription/translation systems can be employed to produce polypeptides encoded by nucleic acids. A number of suitable in vitro transcription and translation systems are commercially available. A general guide to in vitro transcription and translation protocols is found in Tymms (1995) In vitro Transcription and Translation Protocols: Methods in Molecular Biology Volume 37, Garland Publishing, NY.

In addition, polypeptides (including, e.g., polypeptides comprising fluorophores and/or unnatural amino acids) can be produced manually or by using an automated system, by direct peptide synthesis using solid-phase techniques (see, e.g., Chan and White, Eds., (2000) Fmoc Solid Phase Peptide Synthesis: A Practical Approach, Oxford University Press, New York, N.Y.; Lloyd-Williams, P. et al. (1997) Chemical Approaches to the Synthesis of Peptides and Proteins, CRC Press; Stewart et al. (1969) Solid-Phase Peptide Synthesis, WH Freeman Co, San Francisco; Merrifield J (1963) J. Am. Chem. Soc. 85:2149-2154; see also Examples 1 and 3 herein). Exemplary automated systems include the Applied Biosystems 431A Peptide Synthesizer (Perkin Elmer, Foster City, Calif.). In addition, there are many commercial providers of peptide synthesis services. If desired, subsequences can be chemically synthesized separately, and combined using chemical methods to provide full-length polypeptides.

### Production of Aptamers and Antibodies

Aptamers can be selected, designed, etc. for binding various ligands (e.g., substrates in a first or second state) by methods known in the art. For example, aptamers are reviewed in Sun S. “Technology evaluation: SELEX, Gilead Sciences Inc.” Curr Opin Mol Ther. 2000 February; 2(1):100-5; Patel D J, Suri A K. “Structure, recognition and discrimination in RNA aptamer complexes with cofactors, amino acids, drugs and aminoglycoside antibiotics” J Biotechnol. 2000 Mar, 74(1):39-60; Brody E N, Gold L. “Aptamers as therapeutic and diagnostic agents” J Biotechnol. 2000 Mar, 74(1):5-13; Hermann T, Patel D J. “Adaptive recognition by nucleic acid aptamers” Science 2000 Feb. 4, 287(5454):820-5; Jayasena S D. “Aptamers: an emerging class of molecules that rival antibodies in diagnostics” Clin Chem. 1999 Sep, 45(9):1628-50; and Famulok M, Mayer G. “Aptamers as tools in molecular biology and immunology” Curr Top Microbiol Immunol. 1999, 243:123-36.

Antibodies, e.g., that recognize the first or second state of a substrate, can likewise be generated by methods known in the art. For the production of antibodies to a particular polypeptide (e.g., for use as a detection module), various host animals may be immunized by injection with the polypeptide or a portion thereof. Such host animals include, but are not limited to, rabbits, mice and rats, to name but a few. Various adjuvants may be used to enhance the immunological response, depending on the host species; adjuvants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a protein or an antigenic functional derivative thereof. For the production of polyclonal antibodies, host animals, such as those described above, may be immunized by injection with the protein, or a portion thereof, supplemented with adjuvants as also described above. The protein can optionally be produced and purified as described herein. For example, recombinant protein can be produced in a host cell, or a synthetic peptide derived from the sequence of the protein can be conjugated to a carrier protein and used as an immunogen. Standard immunization protocols are described in, e.g., Harlow and Lane (1988) Antibodies, A Laboratory Manual, Cold Spring Harbor Publications, New York. Additional references and discussion of antibodies is also found herein.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature 256:495-497, 1975; and U.S. Pat. No. 4,376,110), the human B-cell hybridoma technique (Kosbor et al. (1983) Immunology Today 4:72; Cole et al. (1983) Proc. Natl. Acad. Sci. USA 80:2026-2030), and the EBV-hybridoma technique (Cole et al. (1985) Monoclonal Antibodies and Cancer Therapy, Alan R. Liss, Inc., pp. 77-96). Such antibodies may be of any immunoglobulin class, including IgG, IgM, IgE, IgA, IgD, and any subclass thereof. The hybridoma producing the mAb of this invention may be cultivated in vitro or in vivo.

In addition, techniques developed for the production of “chimeric antibodies” (Morrison et al. (1984) Proc. Natl. Acad. Sci. USA 81:6851-6855; Neuberger et al. (1984) Nature 312:604-608; Takeda et al. (1985) Nature 314:452-454) by splicing the genes from a mouse antibody molecule of appropriate antigen specificity together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Similarly, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

In addition, techniques described for the production of single-chain antibodies (U.S. Pat. No. 4,946,778; Bird (1988) Science 242:423-426; Huston et al. (1988) Proc. Natl. Acad. Sci. USA 85:5879-5883; and Ward et al. (1989) Nature 334:544-546) can be used. Single chain antibodies are formed by linking the heavy and light chain fragments of the Fv region via an amino acid bridge, resulting in a single-chain polypeptide.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al. (1989) Science 246:1275-1281) to allow rapid and easy identification of monoclonal Fab fragments with the desired specificity.

A large number of antibodies are commercially available. For example, monoclonal and/or polyclonal antibodies against any of a large number of specific proteins (both modified, e.g., phosphorylated, and unmodified), against phosphoserine, against phosphothreonine, against phosphotyrosine, and against any phosphoprotein (i.e., against phosphoserine, phosphothreonine and phosphotyrosine) are available, for example, from Zymed Laboratories, Inc. (on the world wide web at zymed.com), QIAGEN, Inc. (on the world wide web at qiagen.com) and BD Biosciences (on the world wide web at bd.com), among many other sources. In addition, a number of companies offer services that produce antibodies against the desired antigen (e.g., a protein supplied by the customer or a peptide synthesized to order), including Abgent (on the world wide web at abgent.com), QIAGEN, Inc. (on the world wide web at merlincustomservices.com) and Zymed Laboratories, Inc.).

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

### Example 1

### SRC Kinase Sensors

The following sets forth a series of experiments that demonstrate synthesis and use of enzyme sensors (e.g., kinase and phosphatase sensors) including an environmentally sensitive label, a substrate module, and a detection module.

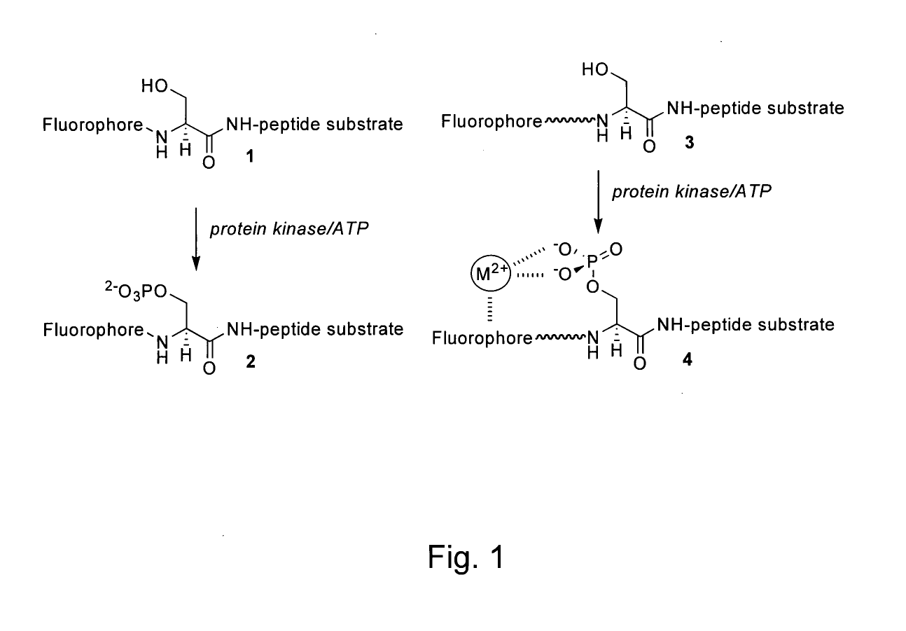

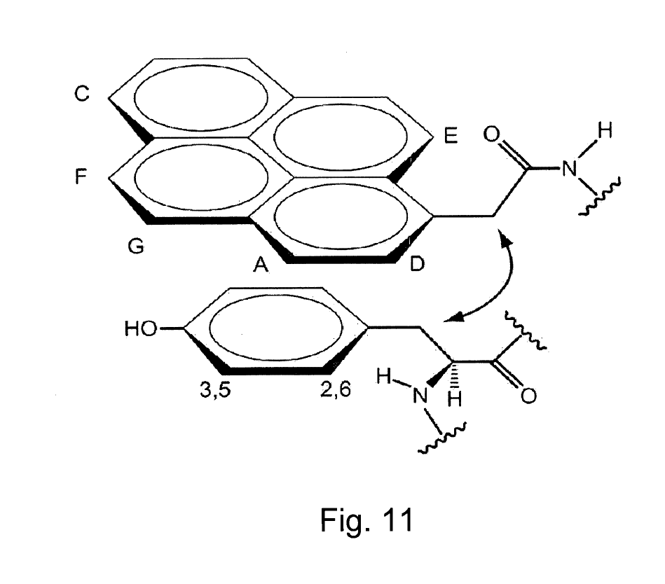

Protein kinases comprise a large family of signaling enzymes that enable the cell to respond to both extracellular and intracellular environmental events. Although the general role played by these enzymes is well recognized, the contributions made by individual protein kinases to specific cellular actions has proven difficult to decipher. In particular, a not uncommon problem is the inability to directly correlate kinase action with some given cellular event of interest. Recently, however, several fluorescent reporters of protein kinase activity have been described, thereby enabling observation of the activity of these enzymes within the context of cellular behavior. Two general strategies have emerged for the design of kinase fluorescent indicators. Several investigators have described genetically encoded proteins comprised of a protein kinase phosphorylation sequence fused to a FRET pair of two spectrally distinct analogs of green fluorescent protein (GFP) (Nagai et al. (2000) Nat. Biotechnol. 18:313-6; Kurokawa et al. (2001) J. Biol. Chem. 276:31305-10; Zhang et al. (2001) Proc. Natl. Acad. Sci. USA 98:14997-5003; Ting et al. (2001) Proc. Natl. Acad. Sci. USA 98:15003-8; Sato et al. (2002) Nat. Biotechnol. 20:287-94; and Violin et al. (2003) C. J. Cell. Biol. 161:899-909). Protein kinase catalyzed phosphorylation of the GFP₁-(protein kinase phosphorylation sequence)-GFP₂ substrate induces FRET changes up to 30%. A second group of kinase probes are comprised of fluorescently-labeled peptides that, upon phosphorylation, display fluorescence changes that are as much as several fold in magnitude. The latter include peptides containing an environmentally sensitive fluorophore directly appended to the phosphorylatable residue (e.g. FIG. 11→2; Yeh et al. (2002) J. Biol. Chem. 277:11527-11532) as well as divalent metal-ion-dependent constructs (e.g. FIG. 13→4; Chen et al. (2002) J. Amer. Chem. Soc. 1243840-3841 and Shults and Imperiali (2003) J. Amer. Chem. Soc. 125:14248-14249). However, the strategies depicted by both 1 and 3 in FIG. 1 lack generality to the protein kinase family and their substrates as a whole, since the peptide-appended fluorophore occupies a fixed spatial relationship with respect to the residue that suffers phosphorylation. This example illustrates a new strategy to sense protein kinase activity that eliminates the need for spatial constraints within the active site-directed peptide substrate. Furthermore, several different fluorophores can be employed with this strategy.

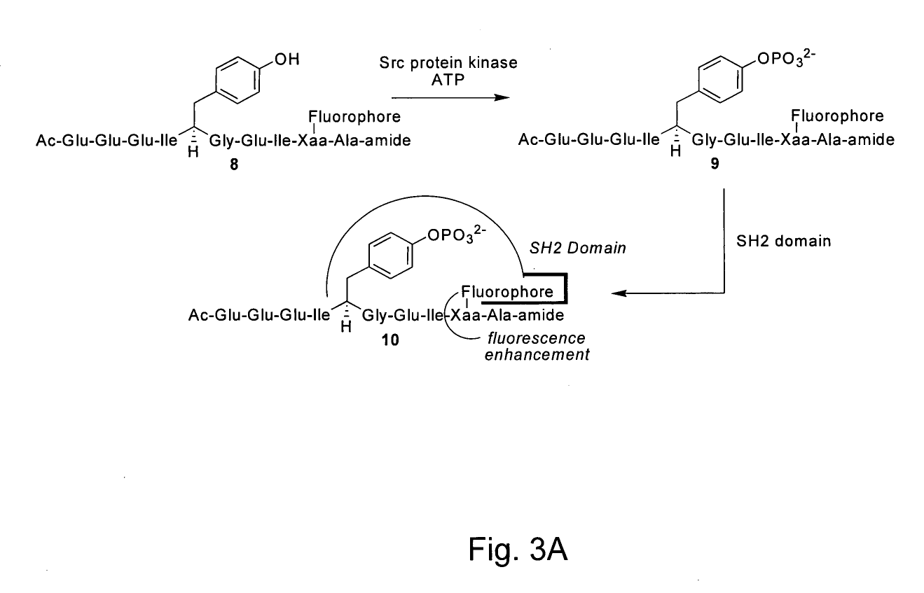

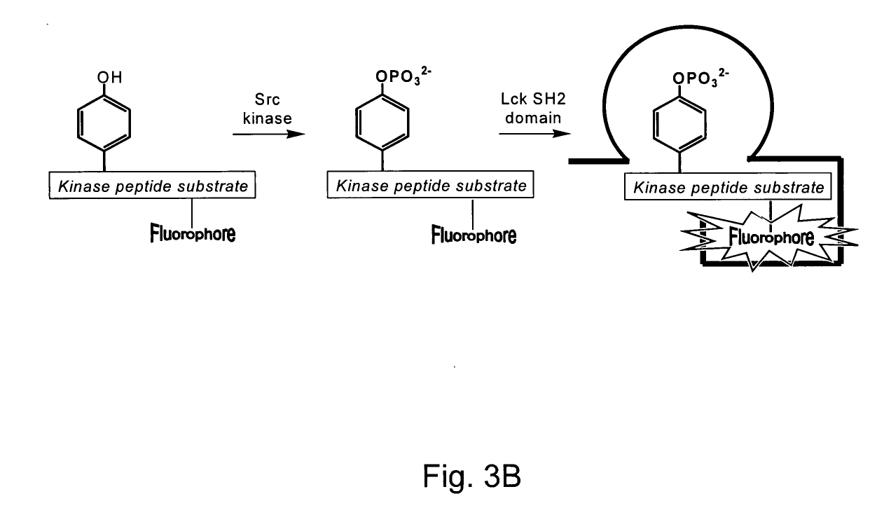

A number of environmentally sensitive fluorophores, such as 5-7 (FIG. 2), have been described. For example, the dapoxyl derivative 5 displays both a shift in its emission wavelength as well as an enhancement in fluorescence quantum yield as a function of decreasing solvent polarity (Diwu et al. (1997) Photochem. Photobiol. 66:424-431). As demonstrated in this example, a fluorescently labeled protein kinase peptide substrate can recapitulate these attributes in an aqueous milieu if, following phosphorylation, the peptide becomes embedded within a hydrophobic environment (FIG. 3 Panels A and B). Several protein-binding domains are known that recognize phosphorylated serine- and tyrosine-containing sequences, including 14-3-3 (see, e.g., Yaffe (2002) FEBS Lett. 513:53-57) and SH2 (see, e.g., Bradshaw and Waksman (2002) Adv. Protein Chem. 61:161-210) domains, respectively. This example illustrates the ability of the Lck SH2 domain to bind to the Src kinase phosphotyrosine peptide product 9 and thereby selectively enhance fluorescent intensity relative to its unphosphorylated counterpart 8 (FIG. 3 Panel A) by providing a relatively hydrophobic environment for the fluorophore.

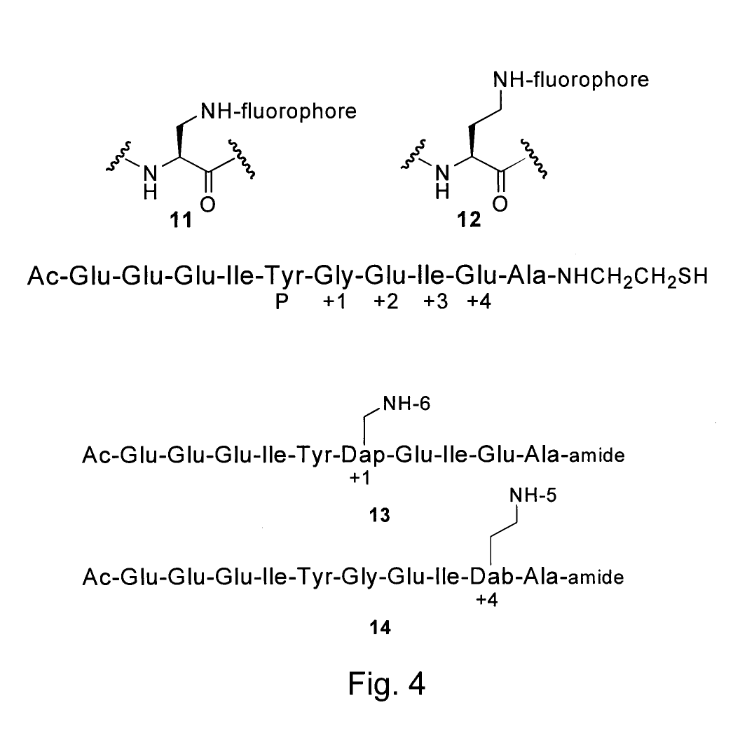

The 3-dimensional structures of several Lck/phosphopeptide complexes have been described (Tong et al. (1996) J. Mol. Biol. 256:601-610 and Mikol et al. (1995) J. Mol. Biol. 246:344-355). Although molecular modeling highlighted a number of potential binding pockets that could offer a relatively lipophilic environment, to ascertain where the fluorophore should be appended on the Peptide in order to ensure SH2-induced fluorescence enhancement while maintaining efficient Src kinase-catalyzed phosphorylation, a library of peptides was prepared in which the three fluorophores 5-7 were attached to (L)-2,3-diaminopropionic acid (Dap) 11 and (L)-2,4-diaminobutyric acid (Dab) 12 (FIG. 4). These six distinct fluorophore-Dap/Dab residues were positioned at four different sites along the peptide backbone (positions P+1−P+4, FIG. 4). (Note that the residues on the N-terminal side of position P (positions P−1−P−4) facilitate Tyr phosphorylation by Src kinase but may not interact with the SH2 domain. The fluorophore can be positioned at any of these sites instead (e.g., at P−2), although the change in fluorescence upon binding of the phosphorylated substrate to the Lck SH2 domain is not as striking.)

The library was prepared via parallel synthesis using a previously described disulfide-linked Tentagel resin (see “Synthesis of Peptide Library” below). Following solid phase synthesis of the primary sequence, the side chain amine of the Dap or Dab residue was selectively deprotected and subsequently modified with the appropriate activated forms of 5, 6, and 7. The remaining protecting groups on the peptide were then removed with trifluoroacetic acid (TFA), the peptide-resin extensively washed to eliminate the last traces of TFA, and the peptide cleaved from the resin with assay buffer (which contained dithiothreitol) and purified by HPLC. The fluorescent response of the individual library members to Src catalysis in the presence of Lck SH2 was subsequently examined in detail (see “Assay of Library” below).

TABLE 1
Fold change in fluorescence intensity in the Src kinase-catalyzed
phosphorylation of peptide substrates as a function of fluorophore
attachment site.

| | FLUOROPHORE ATTACHMENT SITE | | | |
|---|---|---|---|---|
| FLUOROPHORE | +1 | +2 | +3 | +4 |
| Dap-5 | 0.6 | 2.4 | 3.3 | 2.3 |
| Dap-6 | 1.6 (1.6)^{a} | NC^{b} | 1.3 | 1.3 |
| Dap-7 | 1.4 | 1.8 | 1.6 | 1.4 |
| Dab-5 | 2.4 | 1.6 | 3.6 | 4.1 (7.2)^{a} |
| Dab-6 | 1.3 | 1.4 | 1.9 | 1.7 |
| Dab-7 | 1.5 | 1.7 | 2.1 | 1.6 |

^{a}All peptides contain the C-terminal —NH(CH₂)₂SH moiety, except for the —NH2 derivatives indicated by parentheses.
^{b}No change in fluorescence.

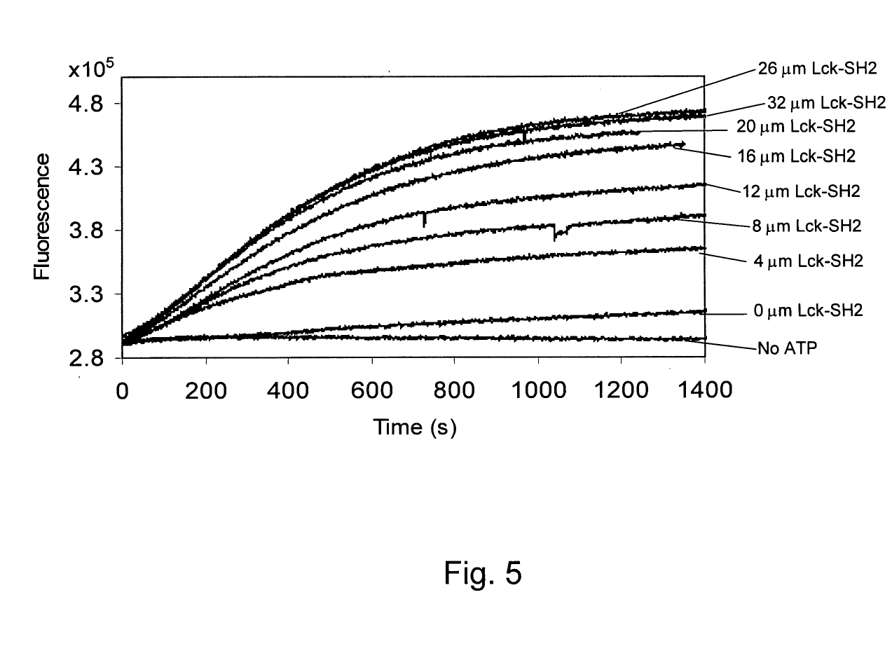

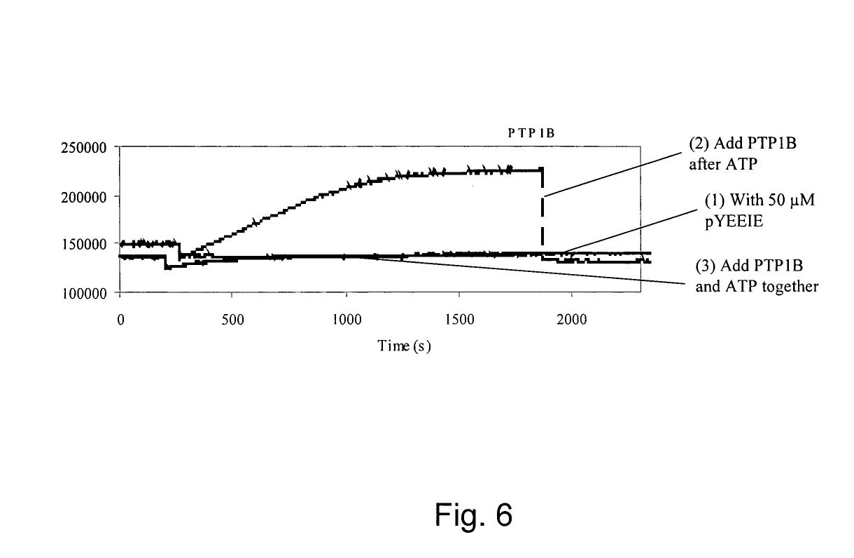

As is evident from Table 1, the dapoxyl fluorophore positioned off the +3 and +4 sites on the peptide substrate (Dap-5 and Dab-5) produce the largest changes in fluorescent behavior. Two peptides (13 and 14, FIG. 4) were examined in greater detail. Both peptides were resynthesized on the Rink resin and purified by HPLC. In addition, the phosphotyrosine version of 13 was enzymatically prepared. The K_{D} of the peptide 13/Lck SH2 domain complex is 2.1±0.2 μM. If the SH2 domain is responsible for the fluorescence change induced by Src kinase-catalyzed phosphorylation, then the Lck SH2 domain concentration should influence the observed fluorescence response. This experiment was performed by fixing the peptide concentration at 16 μM and varying the Lck SH2 domain concentration from 0 to 32 μM (FIG. 5). The reactions were initiated by the addition of ATP. When only buffer was added to “initiate” the reaction (i.e. no ATP), the fluorescence of the mixture remained unperturbed. Furthermore, in the absence of Lck SH2 domain, ATP addition to initiate the reaction furnished an exceedingly modest change in fluorescence intensity (<5%). By contrast, increasing concentrations of SH2 domain produced increasing enhancements in fluorescence intensity. Above an Lck SH2 concentration of 16 μM, the change in fluorescence intensity began to level off, which is in keeping with the notion that the interaction between phosphopeptide and Lck SH2 domain was approaching saturation. In addition, no fluorescence change was observed when the reaction was performed in the presence of the known Lck SH2 domain ligand Ac-pTyr-Glu-Glu-Ile-Glu-amide (SEQ ID NO:8) (50 μM) (FIG. 6; see “Effect of PTP1B and competing Lck-SH2 domain ligand on the fluorescence change” below). This suggests that the fluorophore-appended phosphorylated peptide is binding to the known ligand binding site of the Lck SH2 domain. Furthermore, addition of PTP1B, a phosphotyrosine phosphatase, to the reaction at the same time as ATP blocked the fluorescence change. Finally, addition of PTP1B after completion of the Src kinase-catalyzed reaction reduced the fluorescence intensity to the starting value (FIG. 6; see “Effect of PTP1B and competing Lck-SH2 domain ligand on the fluorescence change” below). These experiments demonstrate that the phosphorylation status of the peptide is essential for the observed change in fluorescence as is the presence of the Lck SH2 domain. Interestingly, when an analogous series of experiments were performed with the amide-capped peptide 14, the observed fluorescence change (7.2-fold) was significantly larger than that exhibited by its library counterpart (4.1-fold). This appears to be a consequence of the —NH(CH₂)₂SH tail that is present on the library members (but not on the amide-capped peptides, as a consequence of the respective synthesis methods used). Both peptides 13 and 14 exhibit Vₘₐₓ (1.4±0.1 and 1.5±0.1 μmol/min-mg, respectively) and Kₘ (32±0.5 and 4.8±0.8 μM, respectively) values comparable to those the best known Src kinase peptide substrates (Lee and Lawrence (1999) J. Med. Chem. 42:784-787).

In summary, the new protein kinase sensing system described herein offers a number of advantages. For example, the ability to utilize full length peptide substrates in which the fluorophore can be appended to different positions on the peptide framework (e.g., as opposed to using “half” length peptide substrates in which the fluorophore is positioned adjacent to the phosphorylatable residue) enables development of sensing systems for those protein kinases that have relatively demanding sequence specificities. In addition, given the fact that a number of different environmentally sensitive fluorophores with a range of photophysical properties have been described (see, e.g., Toutchkine et al. (2003) Amer. Chem. Soc. 125:4132-4145), orthogonal kinase sensing systems can be generated to enable the simultaneous monitoring of two or more protein kinases.

To enable the initiation of the Src kinase-catalyzed phosphorylation of the labeled substrate to be controlled by light, the tyrosine can be caged with a photolabile caging group, e.g., with 2-nitrobenzyl as described in Tatsu et al. (1996) “Solid-phase synthesis of caged peptides using tyrosine modified with a photocleavable protecting group: Application to the synthesis of caged neuropeptide Y” Biochem Biophys Res Comm 227:688-693. The caged substrate can then be uncaged by exposure to light of an appropriate wavelengths to initiate the reaction.

### Experimental Procedures

Materials and chemicals were obtained from Fisher and Aldrich, except for piperidine, 1-hydroxybenzotriazole (HOBt), benzotriazole-1-yloxytris-pyrrolidinophosphonium hexafluorophosphate (PyBop), N,N,N′,N′-tetramethyl-(succinimido)uranium tetrafluoroborate (TSTU), amino acids, TentaGel and Rink resins, which were obtained from Advanced Chemtech, NovaBiochem or Bachem. Dapoxyl sulfonyl chloride (compound 5, X=Cl) and Cascade Yellow succinimidyl ester (compound 7, X=succinimidyl ester) were obtained from Molecular Probes. NBD-Cl (compound 6, X=Cl) was obtained from Acros. Src kinase and PTP1B enzymes were purchased from Invitrogen. Lck-SH2 plasmid was a gift from Professor Steven Shoelson (Joslin Diabetes Center, Harvard Medical School). Glutathione Sepharose™ gel for protein separation was purchased from Amersham Biosciences.

Synthesis of Peptide Library

Diisopropylethylamine (DIPEA; 5 eq, 6.75 mmol, 2.03 g) was added to the suspension of TentaGel S COOH (90 μm, 1 eq, 5 g, 0.27 mmol/g, 1.35 mmol) in 15 mL of DMF containing TSTU (5.0 eq, 2.03 g) and shaken for 10-15 min at room temperature. Then a solution of cystamine dihydrochloride (10 eq, 13.5 mmol, 3.09 g) and DIPEA (20 eq, 27 mmol, 3.49 g) in 15 mL H₂O was carefully added. The mixture was shaken overnight and the resin was washed with H₂O, DMF, and CH₂Cl₂ (each for 3×30 mL). The resulting resin had a free amine substitution of approximately 0.1 mmol/g. The first amino acid, Fmoc-Ala-OH (5 eq, 2.25 mmol, 0.74 g), was attached to the resin using PyBop (5 eq, 1.17 g), HOBt (5 eq, 0.34 g), and DIPEA (10 eq, 0.58 g) in 20 mL DMF for 2 h at room temperature. After washing (3×20 mL DMF, isopropanol and CH₂Cl₂) and drying, the substitution was determined to be 0.100 mmol/g by treating 5 mg resin with 30% piperidine and observing free Fmoc absorption at 290 nm (compared to a standard curve of Fmoc-Ala-OH in 30% piperidine). Peptides were prepared using an Fmoc solid-phase peptide synthesis protocol. The side chains of Glu and Tyr were protected with t-Bu. A peptide library was prepared by sequentially incorporating either (L)-2,3-diaminopropionic acid (Dap) or (L)-2,4-diaminobutyric acid (Dab) at positions P+1 to P+4 (where P=Tyr) in the consensus sequence, Ac-Glu-Glu-Glu-Ile-Tyr-Gly-Glu-Ile-Glu-Ala (SEQ ID NO:5). The side chain amines of the Dap and Dab residues were protected during peptide synthesis with the acid sensitive 4-methyltrityl (Mtt) group. Following peptide synthesis, 15 mg of each individual peptide-resin in the library was treated with 1% TFA in CH₂Cl₂ to selectively deprotect Dap or Dab. Each peptide-resin construct was then split in three equal parts, and the free amine in each construct covalently labeled with NBD (NBD-Cl 20 eq, DIPEA 20 eq, added separately, in DMF, overnight), Dapoxyl (dapoxyl sulfonyl chloride 3 eq, DIPEA 9 eq, in dry CH₂Cl₂, overnight) or Cascade Yellow (Cascade Yellow succinyl ester 2 eq, DIPEA 2 eq, in DMF, overnight). The peptides were then treated with 50% TFA in CH₂Cl₂, washed, and detached from the resin with assay buffer (20 mM DTT in Tris buffer, pH 7.5). The resulting peptide solutions were directly assayed for their ability to fluorescently report Src kinase activity.

Lck-SH2 Protein Expression

E. coli transformed with the GST Lck-SH2 construct was grown at 37° C. in L.B. medium (Luria Broth Base, 25 g/L) until reaching a OD₆₀₀=0.4-0.6 and then induced with 1 mM IPTG (isopropyl-β-D-thiogalactopyranoside). Cells were collected via centrifugation and subsequently sonicated in the presence of 20 mM PBS (pH 7.3). Lck-SH2 was purified on a Glutathione Sepharose™ column. Pure Lck-SH2 was eluted from the column with 20 mM glutathione, dialyzed against 20 mM Tris, pH 7.5, containing 10% glycerol, and concentrated using an Amicon centrifugal filter.

Assay of Library

To a 75 μL 100 mL Tris buffer (pH 7.5) was added 1.25 μL 0.15 mM peptide stock solution, 15 μL 2 mg/mL (0.05 mM) GST-Lck-SH2 (in 10% glycerol), 3.8 μL 200 mM MgCl₂, 1.5 μL 100 mM MnCl₂, 16 μL H₂O, 6 μL 50 mM DTT, and 0.2 μL 0.58 mg/mL (9 μM) Src. The fluorescence of the solution was monitored on a Photon Technology QM-1 spectrofluorimeter at 30° C. at the appropriate excitation and emission wavelengths (NBD peptides: Excitation=470 nm, Emission=530 nm; dapoxyl peptides: Excitation=390 nm, Emission=520 nm; Cascade Yellow peptides: Excitation=400 nm, Emission=535 nm). The fluorescence of the mixture was allowed to stabilize, and then Src kinase-catalyzed phosphorylation was initiated by addition of 15 μL of 10 mM ATP. The final concentration was: 1.25 μM peptide, 5 μM Lck-SH2, 12 nM Src, 1 mM ATP in a buffer containing 50 mM Tris, 5 mM MgCl₂ 1 mM MnCl₂, 2 mM DTT at pH 7.5. The fluorescence change was monitored as a function of time. Control assays in the absence of Lck-SH2 were also performed.

Synthesis of Peptide 14 (P+4 Dab-dapoxyl)

Synthesis of a large quantity of Ac-Glu-Glu-Glu-Ile-Tyr-Gly-Glu-Ile-Dab(dapoxyl)-Ala (peptide 14, SEQ ID NO:7) was performed on the Rink amide resin (0.85 g) following a standard Fmoc solid-phase peptide synthesis protocol using PyBop/HOBt as the coupling reagent. Generally, each coupling was performed at room temperature for 2 h with 5 eq of amino acids, PyBop, HOBt, and 10 eq of DIPEA. However, the coupling of the amino acid immediately after Ile was effected via initial exposure to the standard coupling conditions (i.e. with HOBt and PyBop), followed by a subsequent treatment with the amino acid to be coupled in the presence of HOAt and HATU. Following incorporation of the N-terminal amino acid, the resin was dried and the substitution level determined using the Fmoc absorption method described above (0.12 mmol/g). The free N-terminus was subsequently acetylated. The dried resin (460 mg, 55 μmol) was treated with 1% TFA/CH₂Cl₂ four times, (3 min each), washed (2×CH₂Cl₂, 3× isopropyl alcohol, 3×DMF, and 2×CH₂Cl₂), dried over vacuum, and reacted with 20 mg of dapoxyl sulfonyl chloride (1 eq, 55 μmol) and 21 mg DIPEA (3 eq) in dry CH₂Cl₂ overnight. The peptide was cleaved from the resin (95% TFA, 2.5% triisopropylsilane, 2.5% H₂O), and purified by preparative HPLC (Waters Atlantis dC₁₈ 19×100 mm) using a binary solvent system (solvent A: 1% TFA/H₂O; solvent B: 1% TFA/CH₃CN) with a ratio of A:B that varied from 97:3 (0 min) to 75:25 (5 min) and then changed in a linear fashion to 65:35 (75 min). C₆₀H₈₃N₁₅O₂₅ Calculated m/z 1413.6. found 1412.5 (M-1).

Synthesis of Peptide 13 (P+1 Dap-NBD)

Synthesis of a large quantity of Ac-Glu-Glu-Glu-Ile-Tyr-Dap(NBD)-Glu-Ile-Glu-Ala (peptide 13, SEQ ID NO:6) was performed on the Rink amide resin following a similar procedure described above for peptide 14, except for coupling with NBD: 10 eq. of NBDCl and 10 eq. of DIPEA (added separately) were used with DMF as the solvent. The peptide was purified as described above for compound 14. C₆₉H₉₄N₁₄O₂₃S Calculated m/z 1418.6. found 1517.4 (M-1).

Fluorescence Change as a Function of Lck-SH2 Concentration

The assay protocol described above was used to assess the effect of GST-Lck-SH2 concentration on the observed fluorescence associated with the phosphorylation of pure peptide 13 (30 nM Src) and peptide 14 (15 nM Src).

TABLE 2
Fluorescence change as a function of Lck-SH2 concentration for peptides
13 and 14.

| Peptide 13 | | Peptide 14 | |
|---|---|---|---|
| P + 1 Dap-NBD (16 μM) | | P + 4 Dab-Dapoxyl (8 μM) | |
| Lck-SH2 | Fluorescence Increase | Lck-SH2 | Fluorescence Increase |
| (μM) | (fold change) | (μM) | (fold change) |
| 0 | 1.07 | 0.2 | 1.07 |
| 4 | 1.24 | 2 | 1.70 |
| 8 | 1.33 | 4 | 2.32 |
| 12 | 1.41 | 8 | 4.31 |
| 16 | 1.52 | 12 | 5.34 |
| 20 | 1.56 | 16 | 6.20 |
| 26 | 1.61 | 20 | 6.85 |
| 32 | 1.59 | 26 | 7.32 |
| | | 32 | 7.20 |

K_{d} Determination Compound 13

Control experiments indicated that there is little or no fluorescence change associated with the Src kinase-catalyzed phosphorylation of peptides in the absence of the Lck-SH2 domain. Therefore

ΔF=Q_{b}[PS]+Qₛ([S]ₜ−[PS])+F_{bkg}−(Qₛ[S]ₜ+F_{bkg})=(Q_{b}−Qₛ)[PS]=ΔQ[PS]

in which Q_{b} is the relative quantum yield of bound substrate, Qₛ is the relative quantum yield of free substrate, ΔQ is the difference between Q_{b} and Qₛ, [PS] is the concentration of bound substrate, [S]ₜ is total concentration of phosphorylated peptide, which is assumed to be 16 μM upon phosphorylation, F_{bkg} is the background fluorescence. Combining the equation with K_{d}=([P]ₜ−[PS])([S]ₜ−[PS])/[PS], K_{d} was determined via nonlinear regression analysis using data from assays by fixing peptide concentration and varying GST-Lck-SH2 concentration. The K_{d} determined is 2.1±0.2 μM.

Vₘₐₓ and Kₘ of Compounds 13 and 14

Vₘₐₓ and Kₘ values were determined following the assay protocol described above at a fixed Lck-SH2 concentration of 20 μM and varying peptide concentrations. The final Src concentration was 30 nM for peptide 13 and 15 nM for peptide 14. Peptide 13: Vₘₐₓ=1.4±0.1 μmol/min·mg, Kₘ=32±0.5 μM. Peptide 14: Vₘₐₓ=1.5±0.1 μmol/min·mg, Kₘ=4.8±0.8 μM.

Effect of PTP1B and Competing Lck-SH2 Domain Ligand on the Fluorescence Change

The fluorescence enhancement due to GST-Lck-SH2 was further confirmed with the following experiments (FIG. 6) in the presence of peptide substrate (4 μM), GST-Lck-SH2 (22 μM) and (1) Ac-pTyr-Glu-Glu-Ile-Glu-OH (SEQ ID NO:8; 50 μM), a known Lck-SH2 ligand (no fluorescence change); (2) the addition of the protein phosphatase PTP1B after complete phosphorylation (fluorescence change followed by elimination of the fluorescent enhancement upon PTP1B addition); and (3) the simultaneous addition of PTP1B with ATP (no fluorescent change).

### Example 2

### Exemplary Kinase and Phosphatase Sensors

Table 3 provides additional exemplary kinase and phosphatase sensors. Each sensor includes a detection module (e.g., an SH2 or WW domain) and a polypeptide substrate. An environmentally sensitive fluorescent label (e.g., any of those described or referenced herein) is attached to the polypeptide substrate. If desired, optimal placement of the environmentally sensitive label is determined as described in Example 1, by constructing a library of sensors comprising the label at various positions on the substrate and testing each sensor to determine which sensor(s) produces maximal signal change from the label upon phosphorylation or dephosphorylation of the substrate and consequent association or dissociation of the detection module.

TABLE 3

Exemplary sensor components, including for each sensor
a detection module (detect. module), the amino acid
sequence of the polypeptide substrate, with the
residue modified (phosphorylated or dephosphorylated)
by the enzyme identified by its position in the
substrate and its name (phos. residue), and the
corresponding enzyme identified by its Swiss-Prot
accession number (access. number), name, and type
(kinase or phosphatase). The Swiss-Prot database is
available, e.g., on the internet at au.expasy.org/sprot.

| | | | | | | |
|---|---|---|---|---|---|---|
| detect. | polypeptide | SEQ ID | phos. | access. | enzyme | enzyme |
| module | substrate | NO: | residue | number | name | type |

| | | | | | | | |
|---|---|---|---|---|---|---|---|
| SH2 | LLDKYLIPNATQ | 21 | 5 | Y | P31946 | 143B_HUMAN | Kinase |
| WW | YEILNSPEKACS | 22 | 6 | S | P29312 | 143Z_HUMAN | Kinase |
| WW | LTLKKTPGRSTGE | 23 | 6 | T | Q92790 | MOK_HUMAN | Kinase |
| SH2 | VNPYYLRVRRKN | 24 | 5 | Y | Q13131 | AAK1_HUMAN | Kinase |
| WW | HGGHKTPRRDSSG | 25 | 6 | T | Q9Y478 | AAKB_HUMAN | Kinase |
| WW | LTPEKSPKFPDSQ | 26 | 6 | S | Q9UKA4 | AK11_HUMAN | Kinase |
| SH2 | SGGLELYGEPRHTT | 27 | 7 | Y | Q99996 | AKA9_HUMAN | Kinase |
| SH2 | MHSVYQPQPSASQ | 28 | 5 | Y | Q9NSY1 | BM2K_HUMAN | Kinase |
| SH2 | LWEAYANLHTAV | 29 | 5 | Y | P51813 | BMX_HUMAN | Kinase |
| WW | RSNPKSPQKPIVR | 30 | 6 | S | P15056 | BRAF_HUMAN | Kinase |
| WW | LRRDKSPGRPLER | 31 | 6 | S | O14578 | CTRO_HUMAN | kinase |
| WW | LEREKSPGRMLST | 32 | 6 | S | O14578 | CTRO_HUMAN | kinase |
| SH2 | DSTAETYGKIVHYK | 33 | 7 | Y | Q09013 | DMK_HUMAN | Kinase |
| WW | KAEEKSPKKQKVT | 34 | 6 | S | Q9NR20 | DYR4_HUMAN | Kinase |
| WW | TVWKKSPEKNERH | 35 | 6 | S | P19525 | E2K2_HUMAN | Kinase |
| SH2 | EEMTYEEIQEHY | 36 | 5 | Y | P16118 | F261_HUMAN | Kinase |
| SH2 | VESIYLNVEAVN | 37 | 5 | Y | P16118 | F261_HUMAN | Kinase |
| SH2 | EELTYEEIRDTY | 38 | 5 | Y | Q16875 | F263_HUMAN | Kinase |
| SH2 | EEMTYEEIQDNY | 39 | 5 | Y | Q16877 | F264_HUMAN | Kinase |
| SH2 | PPEEYVPMVKEV | 40 | 5 | Y | Q05397 | FAK1_HUMAN | Kinase |
| SH2 | FSSSEIYGLIKTGA | 41 | 7 | Y | Q14410 | GKP2_HUMAN | Kinase |
| SH2 | GTVGYMAPEVVK | 42 | 5 | Y | P43250 | GRK6_HUMAN | Kinase |
| SH2 | QKYAYLNVVGMV | 43 | 5 | Y | Q01813 | K6PP_HUMAN | Kinase |
| SH2 | LGTEELYGYLKKYH | 44 | 7 | Y | P19784 | KC22_HUMAN | Kinase |
| SH2 | VLRKEAYGKPVDIW | 45 | 7 | Y | Q13554 | KCCB_HUMAN | Kinase |
| SH2 | MFMWYLNPRQVF | 46 | 5 | Y | O75912 | KDGI_HUMAN | Kinase |
| SH2 | KDEVYLNLVLDY | 47 | 5 | Y | P49841 | KG3B_HUMAN | Kinase |
| SH2 | ELLTELYGKVGEIR | 48 | 7 | Y | P46020 | KPB1_HUMAN | kinase |
| WW | RDGYKTPKEDPNR | 49 | 6 | T | P46020 | KPB1_HUMAN | Kinase |
| SH2 | NLLGELYGKAGLNQ | 50 | 7 | Y | P46019 | KPB2_HUMAN | Kinase |
| WW | RDGYKTPREDPNR | 51 | 6 | T | P46019 | KPB2_HUMAN | Kinase |
| SH2 | EGFSYVNPQFVH | 52 | 5 | Y | P17252 | KPCA_HUMAN | Kinase |
| WW | RPKVKSPRDYSNF | 53 | 6 | S | Q05655 | KPCD_HUMAN | Kinase |
| SH2 | KFNGYLRVRIGE | 54 | 5 | Y | P24723 | KPCL_HUMAN | Kinase |
| SH2 | VWVDYPNTVRVV | 55 | 5 | Y | P30613 | KPYR_HUMAN | Kinase |
| SH2 | GTAAYMAPEVIT | 56 | 5 | Y | Q9Y6R4 | M3K4_HUMAN | Kinase |
| SH2 | GTLQYMAPEIID | 57 | 5 | Y | Q96B75 | M3K6_HUMAN | Kinase |
| SH2 | ENIAELYGAVLWGE | 58 | 7 | Y | P41279 | M3K8_HUMAN | kinase |
| WW | PNLGKSPKHTPIA | 59 | 6 | S | Q02779 | M3KA_HUMAN | Kinase |
| WW | VGGLKSPWRGEYK | 60 | 6 | S | Q99558 | M3KE_HUMAN | Kinase |
| WW | VTLTKSPKKRPSA | 61 | 6 | S | Q92918 | M4K1_HUMAN | Kinase |
| SH2 | LQHPYINVWYDPA | 62 | 5 | Y | P53779 | MK10_HUMAN | Kinase |
| SH2 | GTRSYMAPERLQ | 63 | 5 | Y | P36507 | MPK2_HUMAN | Kinase |
| SH2 | GCRPYMAPERID | 64 | 5 | Y | P45985 | MPK4_HUMAN | Kinase |
| SH2 | GTNAYMAPERIS | 65 | 5 | Y | Q13163 | MPK5_HUMAN | Kinase |
| SH2 | GCKPYMAPERIN | 66 | 5 | Y | P52564 | MPK6_HUMAN | Kinase |
| SH2 | GCAAYMAPERID | 67 | 5 | Y | O14733 | MPK7_HUMAN | Kinase |
| SH2 | AAYCYLRVVGKG | 68 | 5 | Y | P51957 | NEK4_HUMAN | Kinase |
| SH2 | GDPRYMAPELLQ | 69 | 5 | Y | O14731 | PMYT1_HUMAN | Kinase |
| WW | PVPKKSPKSQPLE | 70 | 6 | S | O43863 | BAIP1_HUMAN | Kinase |
| SH2 | SEDVYTAVEHSD | 71 | 5 | Y | Q9ULU4 | PKCB_HUMAN | Kinase |
| SH2 | QWFREAYGAVTQTV | 72 | 7 | Y | Q15126 | PMVK_HUMAN | Kinase |
| WW | LTWNKSPKSVLVI | 73 | 6 | S | O95544 | PPNK_HUMAN | Kinase |
| SH2 | MSPDYPNPMFEH | 74 | 5 | Y | P78527 | PRKD_HUMAN | Kinase |
| SH2 | KAAGYANPVWTA | 75 | 5 | Y | Q16584 | Q16584 | Kinase |
| WW | QRSAKSPRREEEPR | 76 | 6 | S | Q16584 | Q16584 | Kinase |
| SH2 | ESLVETYGKIMNHK | 77 | 7 | Y | Q86XX2 | Q86XX2 | Kinase |
| SH2 | ESLVETYGKIMNHE | 78 | 7 | Y | Q86XZ8 | Q86XZ8 | Kinase |
| SH2 | GTKPYMAPEVFQ | 79 | 5 | Y | Q8IY14 | Q8IY14 | Kinase |
| WW | LVRSKSPKITYFT | 80 | 6 | S | Q8IYF0 | PLK4_HUMAN | Kinase |
| SH2 | GSPMYMAPEVIM | 81 | 5 | Y | Q8IYT8 | Q8IYT8 | Kinase |
| SH2 | EGFEYINPLLMS | 82 | 5 | Y | Q8WW06 | Q8WW06 | Kinase |
| SH2 | GLQNYLNVITTN | 83 | 5 | Y | Q96CA3 | Q96CA3 | Kinase |
| SH2 | DGNGYISAAELR | 84 | 5 | Y | Q96HY3 | Q96HY3 | Kinase |
| SH2 | YAIKYVNLEEAD | 85 | 5 | Y | Q9BW51 | Q9BW51 | Kinase |
| SH2 | YGDIYLNAGPML | 86 | 5 | Y | Q9H4A0 | Q9H4A0 | Kinase |
| SH2 | KWKMYMEMDGDE | 87 | 5 | Y | Q9HDD2 | Q9HDD2 | Kinase |
| WW | ISNFKTPSKLSEK | 88 | 6 | T | Q9NPD9 | Q9NPD9 | Kinase |
| SH2 | PKSEEPYGQLNPKW | 89 | 7 | Y | Q9NUW2 | Q9NUW2 | Kinase |
| WW | RSIIKTPKTQDTE | 90 | 6 | T | Q9NYJ8 | Q9NYJ8 | Kinase |
| WW | SGRLKTPGKREIPV | 91 | 6 | T | Q9UF33 | Q9UF33 | Kinase |
| SH2 | GSPLYMAPEMVC | 92 | 5 | Y | Q9UFS4 | Q9UFS4 | Kinase |
| SH2 | GTLYYMAPEHLN | 93 | 5 | Y | Q13546 | RIK1_HUMAN | Kinase |
| SH2 | NQETYLNISQVN | 94 | 5 | Y | O94768 | S17B_HUMAN | Kinase |
| WW | PHNPKTPPKSPVV | 95 | 6 | T | O94932 | O94932 | Kinase |
| SH2 | LTHDYINLFHFPG | 96 | 5 | Y | Q9HCC5 | Q9HCC5 | Kinase |
| WW | PANQKSPKGKLRL | 97 | 6 | S | O00757 | F16Q_HUMAN | Phosphatase |
| WW | CENAKTPKEQFRV | 98 | 6 | T | O95172 | O95172 | Phosphatase |
| SH2 | REKEYVNIQTFR | 99 | 5 | Y | Q01968 | OCRL_HUMAN | Phosphatase |
| SH2 | LAKWYVNAKGYF | 100 | 5 | Y | Q13393 | PLD1_HUMAN | Phosphatase |
| SH2 | PDDKYIYVADIL | 101 | 5 | Y | Q15165 | PON2_HUMAN | Phosphatase |
| WW | LRFLESPDFQPS | 102 | 6 | S | Q15173 | 2A5B_HUMAN | Phosphatase |
| WW | LPPASTPTSPSS | 103 | 6 | T | Q15173 | 2A5B_HUMAN | Phosphatase |
| WW | ASTPTSPSSPGL | 104 | 6 | S | Q15173 | 2A5B_HUMAN | Phosphatase |
| WW | PTSPSSPGLSPV | 105 | 6 | S | Q15173 | 2A5B_HUMAN | Phosphatase |
| WW | SSPGLSPVPPPD | 106 | 6 | S | Q15173 | 2A5B_HUMAN | Phosphatase |
| WW | NQQELTPLPLLK | 107 | 6 | T | Q15173 | 2A5B_HUMAN | Phosphatase |
| WW | ARCVSSPHFQVA | 108 | 6 | S | Q15173 | 2A5B_HUMAN | Phosphatase |
| WW | PLQRLTPQVAAS | 109 | 6 | T | Q15173 | 2A5B_HUMAN | Phosphatase |
| WW | ISHEHSPSDLEA | 110 | 6 | S | P30153 | 2AAA_HUMAN | Phosphatase |
| WW | VIMGLSPILGKD | 111 | 6 | S | P30153 | 2AAA_HUMAN | Phosphatase |
| WW | LCSDDTPMVRRA | 112 | 6 | T | P30153 | 2AAA_HUMAN | Phosphatase |
| WW | ISQEHTPVALEA | 113 | 6 | T | P30154 | 2AAB_HUMAN | Phosphatase |
| WW | QLTPFSPVFGTE | 114 | 6 | S | Q06190 | 2ACA_HUMAN | Phosphatase |
| WW | LKKCPTPMQNEI | 115 | 6 | T | Q06190 | 2ACA_HUMAN | Phosphatase |
| WW | KSKVSSPIEKVS | 116 | 6 | S | Q06190 | 2ACA_HUMAN | Phosphatase |
| WW | PIEKVSPSCLTR | 117 | 6 | S | Q06190 | 2ACA_HUMAN | Phosphatase |
| WW | LSVCRSPVGDKA | 118 | 6 | S | Q06190 | 2ACA_HUMAN | Phosphatase |
| WW | VLIQQTPEVIKI | 119 | 6 | T | Q06190 | 2ACA_HUMAN | Phosphatase |
| WW | EKKPGTPLPPPA | 120 | 6 | T | Q06190 | 2ACA_HUMAN | Phosphatase |
| SH2 | SESAYPNAELVF | 121 | 5 | Y | Q13613 | MTR1_HUMAN | Phosphatase |
| SH2 | KEiVYPNIEETH | 122 | 5 | Y | Q13614 | MTR2_HUMAN | Phosphatase |
| WW | AELIKTPRVDNVV | 123 | 6 | T | Q96QG7 | MTR9_HUMAN | Phosphatase |
| SH2 | LTYIYPNIIAMG | 124 | 5 | Y | O00633 | PTEN_HUMAN | Phosphatase |
| SH2 | EDNDYINASLIK | 125 | 5 | Y | P18031 | PTN1_HUMAN | Phosphatase |
| SH2 | AESCYINIARTL | 126 | 5 | Y | P26045 | PTN3_HUMAN | Phosphatase |
| SH2 | GNEDYINANYIN | 127 | 5 | Y | P29074 | PTN4_HUMAN | Phosphatase |
| WW | WPDQKTPDRAPPL | 128 | 6 | T | P54829 | PTN5_HUMAN | Phosphatase |
| SH2 | PGSDYINANYIK | 129 | 5 | Y | P29350 | PTN6_HUMAN | Phosphatase |
| SH2 | EDGDYINANYIR | 130 | 5 | Y | P35236 | PTN7_HUMAN | Phosphatase |
| WW | PPPEKTPAKKHVR | 131 | 6 | T | P35236 | PTN7_HUMAN | Phosphatase |
| SH2 | TQTDYINASFMD | 132 | 5 | Y | P43378 | PTN9_HUMAN | Phosphatase |
| SH2 | KGHEYTNIKYSL | 133 | 5 | Y | Q06124 | PTNB_HUMAN | Phosphatase |
| SH2 | SARVYENVGLMQ | 134 | 5 | Y | Q06124 | PTNB_HUMAN | Phosphatase |
| SH2 | QDSDYTNANFIK | 135 | 5 | Y | Q05209 | PTNC_HUMAN | Phosphatase |
| SH2 | DEGGYINASFIK | 136 | 5 | Y | Q12923 | PTND_HUMAN | Phosphatase |
| WW | KKQCKSPSRRDSY | 137 | 6 | S | Q12923 | PTND_HUMAN | Phosphatase |
| SH2 | LFPIYENVNPEY | 138 | 5 | Y | P23467 | PTPB_HUMAN | Phosphatase |
| SH2 | ARSDYLRVSWVH | 139 | 5 | Y | P23467 | PTPB_HUMAN | Phosphatase |
| SH2 | PCSDYINASYIPG | 140 | 5 | Y | P23467 | PTPB_HUMAN | Phosphatase |
| SH2 | IKGYYIIIVPLK | 141 | 5 | Y | P23468 | PTPD_HUMAN | Phosphatase |
| SH2 | YSIKYTAVDGED | 142 | 5 | Y | P23468 | PTPD_HUMAN | Phosphatase |
| SH2 | EKNRYPNILPND | 143 | 5 | Y | P23469 | PTPE_HUMAN | Phosphatase |
| SH2 | EYTDYINASFID | 144 | 5 | Y | P23469 | PTPE_HUMAN | Phosphatase |
| SH2 | KHSDYINANYVD | 145 | 5 | Y | P23470 | PTPG_HUMAN | Phosphatase |
| SH2 | SRSDYINASPII | 146 | 5 | Y | Q16849 | PTPN_HUMAN | Phosphatase |
| SH2 | SHSDYINASPIM | 147 | 5 | Y | Q92932 | PTPX_HUMAN | Phosphatase |
| SH2 | HKNRYINTVAYD | 148 | 5 | Y | P23471 | PTPZ_HUMAN | Phosphatase |
| SH2 | KLTDYINANYVD | 149 | 5 | Y | P23471 | PTPZ_HUMAN | Phosphatase |
| SH2 | HIHAYVNALLIPG | 150 | 5 | Y | P23471 | PTPZ_HUMAN | Phosphatase |
| SH2 | EGTDYINASYIM | 151 | 5 | Y | P23471 | PTPZ_HUMAN | Phosphatase |
| SH2 | GKDDYINASCVE | 152 | 5 | Y | Q9BSR5 | Q9BSR5 | Phosphatase |
| WW | SYNEKTPRIVVSR | 153 | 6 | T | Q9NX62 | Q9NX62 | Phosphatase |
| SH2 | ALVQYINQLCRH | 154 | 5 | Y | Q9NZS4 | Q9NZS4 | Phosphatase |
| SH2 | CGLPYINLEFLK | 155 | 5 | Y | Q9NZS4 | Q9NZS4 | Phosphatase |
| WW | TVKPKSPEKSKPD | 156 | 6 | S | Q9NZS4 | Q9NZS4 | Phosphatase |
| WW | KDPEKSPTKKQEV | 157 | 6 | S | Q9NZS4 | Q9NZS4 | Phosphatase |
| SH2 | EDSSYINANFIK | 158 | 5 | Y | Q920U2 | Q9P0U2 | Phosphatase |
| WW | KQTLKTPGKSFTR | 159 | 6 | T | Q9P0U2 | Q9POU2 | Phosphatase |

A large number of additional kinases (or phosphatases), substrates, and detection modules can be found in the art. For example, the KinaseProfiler™ Assay Protocols protocol guide from Upstate (October 2003; available on the world wide web at upstate.com/img/pdf/kp_protocols_full.pdf) lists about 100 kinase-substrate combinations (including, e.g., examples of both specific and generic substrates).

In another aspect, additional exemplary kinase and phosphatase sensors can be produced using the substrates noted above, e.g., in Table 3. An environmentally sensitive or fluorescent label (e.g., any of those described or referenced herein) is attached to the polypeptide substrate. If desired, optimal placement of the label is determined as described in Example 3, by constructing a library of sensors comprising the label at various positions on the substrate and testing each sensor to determine which sensor(s) produces maximal signal change from the label upon phosphorylation or dephosphorylation of the substrate. These exemplary sensors do not include a detection module.

### Example 3

### Tyrosine Kinase Sensors

The following sets forth a series of experiments that demonstrate synthesis and use of enzyme sensors (e.g., kinase and phosphatase sensors) including an environmentally sensitive or fluorescent label. The sensors include self-reporting fluorescent substrates and thus do not require the presence of a detection module.

Probes that provide a continuous fluorescent readout of protein tyrosine kinase activity offer a direct means to observe kinase action in living cells, can serve in a diagnostic capacity as sensors of aberrant activity, and can prove invaluable in high throughput screening assays, for example. Several genetically encoded FRET-based proteins have been described that, upon tyrosine phosphorylation, display fluorescent changes up to 50% (Zaccolo (2004) “Use of chimeric fluorescent proteins and fluorescence resonance energy transfer to monitor cellular responses” Circ. Res. 94:866-73). A few peptide-derived reporters have been introduced as well, but these require non-physiological levels of “helper” ions (Shults and Imperiali (2003) “Versatile fluorescence probes of protein kinase activity” J. Am. Chem. Soc. 125:14248-9) or proteins (Wang and Lawrence (2005) “Phosphorylation-driven protein-protein interactions: A protein kinase sensing system” J. Am. Chem. Soc. 127:7684-5) to observe a fluorescent change in response to tyrosine phosphorylation. In contrast, this example describes a strategy that permits a peptide substrate to self-recognize and fluorescently report the phosphorylation of tyrosine residues. This approach has furnished peptide substrates that display a several-fold amplification of fluorescent intensity upon phosphorylation. In addition, these substrates can be conveniently used, e.g., to examine kinase self-activation and activity, e.g., under cellular-mimetic conditions or inside cells, without requiring use of non-physiological levels of divalent cations, detection modules, quenchers, and/or FRET pairs.

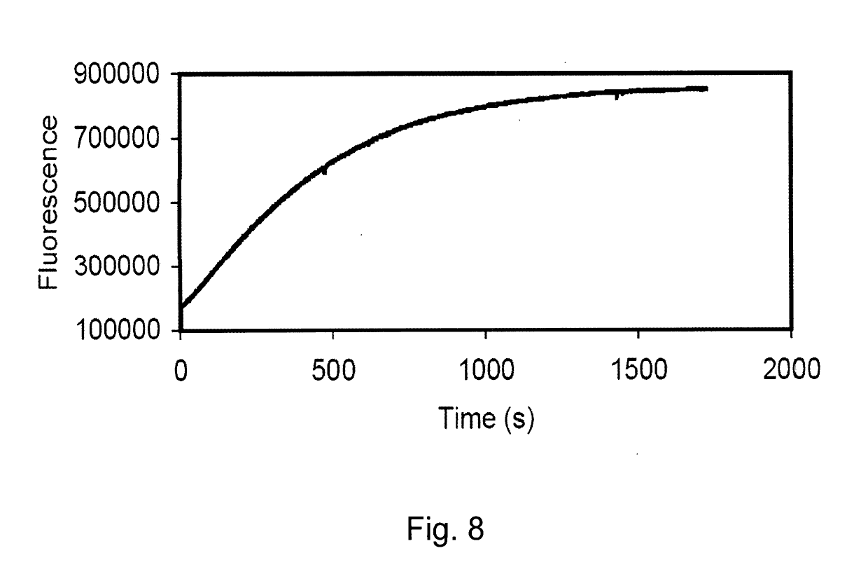

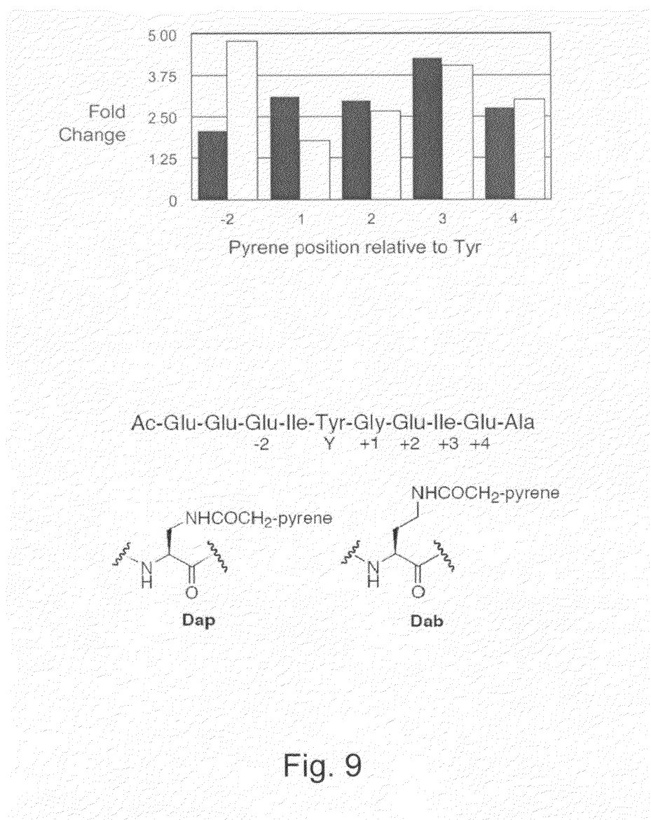

The tyrosine aryl side chain is known to engage other aromatic species, including fluorophores, in, inter alia, π-π stacking interactions (Kraft et al. (2003) “Spectroscopic and mutational analysis of the blue-light photoreceptor AppA: A novel photocycle involving flavin stacking with an aromatic amino acid” Biochemistry 42:6726-34). Phosphorylation of the tyrosine moiety can alter the nature of, or possibly disrupt, these interactions, thereby leading to a perturbation of the photophysical properties of the aromatic binding partner. Pyrene was employed as the aromatic binding partner in this example, since the fluorescent properties of this fluorophore are sensitive to environmental conditions (Schechter et al. (1975) “Structural alterations in the 30 S ribosomal subunit of Escherichia coli observed with the fluorescent probe N-(3-pyrene) maleimide” FEBS Lett. 57:149-52). Src and related tyrosine kinases catalyze the phosphorylation of the tyrosine moiety in acidic peptides, such as Ac-Glu-Glu-Glu-Ile-Tyr-Gly-Glu-Ile-Glu-Ala (SEQ ID NO: 5) (Wang and Lawrence, supra, Porter et al. (2000) “Reciprocal regulation of Hck activity by phosphorylation of Tyr(527) and Tyr(416) Effect of introducing a high affinity intramolecular SH2 ligand” J. Biol. Chem. 275:2721-6, and Songyang et al. (1995) “Catalytic specificity of protein-tyrosine kinases is critical for selective signalling” Nature 373:536-9). A library of analogs of this peptide was prepared in which a pyrene substituent is appended off of (L)-2,3-diaminopropionic acid 21 (Dap) or (L)-2,4-diaminobutanoic acid 22 (Dab) residues at specific sites on the peptide chain encompassing the tyrosine moiety (FIG. 7). Individual members of this library were subsequently incubated with Src and fluorescent intensity followed as a function of time. Phosphorylation-induced changes range from a minimum of 1.8-fold up to nearly 5-fold (FIGS. 8 and 9). Two peptides were chosen for further evaluation, namely the Dap-substituted derivative at Y+3 (23, SEQ ID NO:14) (4.3-fold) and the Dab-modified analogue at Y-2 (25, SEQ ID NO:12) (4.7-fold). The phosphorylated analogue of 23, peptide 24, was synthesized as well.

Both unphosphorylated and phosphorylated peptide derivatives were examined by NMR to assess whether the aromatic moieties of the pyrene and tyrosine residues are spatially proximate. The pyrene protons in the unphosphorylated peptide 23 exhibit pronounced nuclear Overhauser enhancements (NOEs) with their tyrosine counterparts (FIG. 10 Panel A; see Panel C for pyrene proton designations). NOEs between the benzylic protons of the two aryl substituents are present as well. Furthermore, all of the aromatic and benzylic protons on the tyrosine side chain are shifted upfield, suggesting that the pyrene and tyrosine rings are engaged in a π-π stacking interaction as opposed to an edge-face interaction (Hunter et al. (2001) “Aromatic Stacking Interactions” J. Chem. Soc., Perkin Trans. 2:651-69). Without intending to be limited to any particular mechanism, a working model of the interaction between the pyrene and tyrosine aromatic nuclei is schematically illustrated in FIG. 11. In contrast to the results obtained for compound 23, the corresponding phosphorylated peptide 24 exhibits only weak NOEs between the two aryl substituents (FIG. 10 Panel B). These results indicate that the phosphate moiety compromises the ability of the pyrene and tyrosine aryl groups to interact with one another and suggest that the enhanced pyrene fluorescence in 24 is a consequence of its phosphorylation-induced liberated state.

Peptides 23 and 25 serve as substrates for a variety of protein tyrosine kinases (Table 4). Since Src recognizes the chosen peptide sequence, it is not surprising that other members of the Src kinase subfamily (SrcN1, Src N2, Fyn, Fgr, Hck, Lck, Yes, LynA, and LynB) likewise utilize peptides 23 and 25 as substrates. In addition, other non-receptor tyrosine kinases (Abl, Csk, and Fes/Fps) as well as receptor tyrosine kinases (FGFR, TrkA, and Flt3) phosphorylate both peptides. However, these peptides are by no means universal tyrosine kinase substrates since several enzymes (ZAP-70, c-Met, EGF, Eph, IR, MLK1) are unable to effectively catalyze the phosphorylation of either 23 or 25. The amino acid sequence preferences of these noncompliant kinases are likely responsible for this behavior. In general, the phosphorylation of the Y-2 Dab derivative 25 proceeds with modestly lower Kₘ values than its Y+3 counterpart 23. There are a number of possible explanations for the latter observation with perhaps the simplest being that the various tyrosine kinases find the bulky Dap-pyrene moiety at Y+3 slightly more challenging to accommodate.

TABLE 4
Kₘ (μM) and Vₘₐₓ (μmol/min-mg) values for the tyrosine kinase-
catalyzed phosphorylation of peptides 23 and 25.

| Tyrosine | Y + 3 Dab-pyrene (23) | | Y − 2 Dap-pyrene (25) | |
|---|---|---|---|---|
| Kinase | Vₘₐₓ | Kₘ | Vₘₐₓ | Kₘ |
| Src | 5.2 ± 0.4 | 93 ± 8 | 2.4 ± 0.2 | 21 ± 3 |
| SrcN1 | 3.0 ± 0.7 | 225 ± 50 | 3.1 ± 0.4 | 69 ± 10 |
| SrcN2 | 14 ± 2 | 244 ± 30 | 9 ± 1 | 61 ± 10 |
| FynT | 0.24 ± 0.05 | 69 ± 16 | 0.41 ± 0.05 | 24 ± 4 |
| Fgr | 2.3 ± 0.2 | 54 ± 6 | 0.81 ± 0.8 | 30 ± 1 |
| Lck | 1.5 ± 0.2 | 96 ± 10 | 2.1 ± 0.1 | 40 ± 1 |
| Yes | 3.3 ± 0.2 | 37 ± 2 | 1.4 ± 0.1 | 15 ± 2 |
| LynA | 2.6 ± 0.5 | 140 ± 1 | 2.6 ± 0.2 | 43 ± 4 |
| LynB | 4.0 ± 0.7 | 130 ± 10 | 2.9 ± 0.1 | 38 ± 1 |
| Hck | 6.6 ± 0.8 | 170 ± 15 | 3.2 ± 0.5 | 26 ± 0.5 |
| Abl | 0.4 ± 0.2 | 90 ± 10 | 0.44 ± 0.07 | 110 ± 3 |
| Csk | 0.4 ± 0.1 | 120 ± 40 | 2.0 ± 0.2 | 150 ± 20 |
| Fes/Fps | 3.0 ± 0.2 | 60 ± 40 | 4.1 ± 0.2 | 130 ± 10 |
| FGFR | 0.7 ± 0.1 | 150 ± 20 | 0.98 ± 0.09 | 80 ± 10 |
| TrkA | 1.1 ± 0.1 | 350 ± 20 | 2.9 ± 0.5 | 210 ± 40 |
| Flt3 | 4.9 ± 0.8 | 450 ± 30 | 5.0 ± 2.0 | 280 ± 100 |

A fluorescent tyrosine kinase reporter such as those described herein offers a number of distinct advantages relative to conventional fixed time point kinase assays (e.g. [³²P]ATP, ELISA, etc.). Safety concerns associated with the radioactive ATP method preclude the use of ATP concentrations that are present in cells (1-10 mM). Unfortunately, low concentrations of the latter can deceptively inflate the potency of protein kinase inhibitors since the vast majority are competitive with ATP (Lawrence and Niu (1998) “Protein kinase inhibitors: The tyrosine-specific protein kinases” Pharmacol. Ther. 77:81-114). For example, the pyrazolopyrimidine PP2 serves as a general inhibitor of the Src tyrosine kinase family (Hanke et al. (1996) “Discovery of a novel, potent, and Src family-selective tyrosine kinase inhibitor. Study of Lck- and FynT-dependent T cell activation” J. Biol. Chem. 271:695-701 and Bain et al. (2003) “The specificities of protein kinase inhibitors: An update” Biochem. J. 371:199-204). In contrast to the radioactive assay employed in the latter studies, physiologically relevant ATP concentrations can be readily used with the pyrene-peptide substrates. Using a pyrene-peptide substrate, the IC₅₀ of PP2 at 5 mM ATP is determined to be 4.1±0.3 μM (Lck kinase), approximately 50-fold higher than the corresponding IC₅₀ (86±14 nM) at 50 μM ATP. These results confirm that ATP levels have a clear impact on the apparent efficacy of inhibitors that are competitive with ATP.

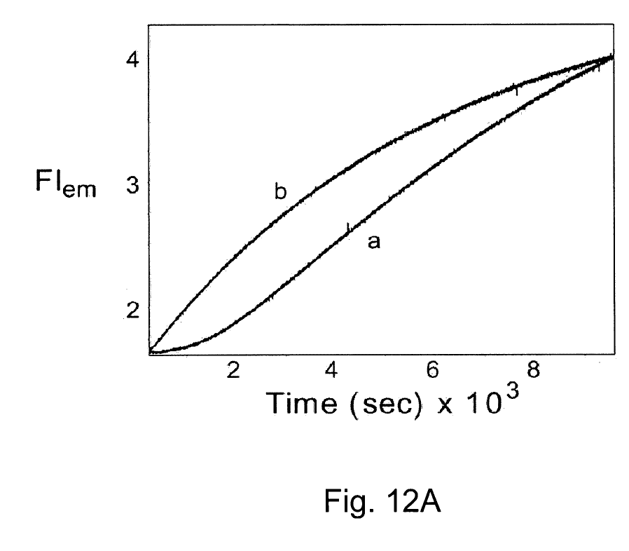

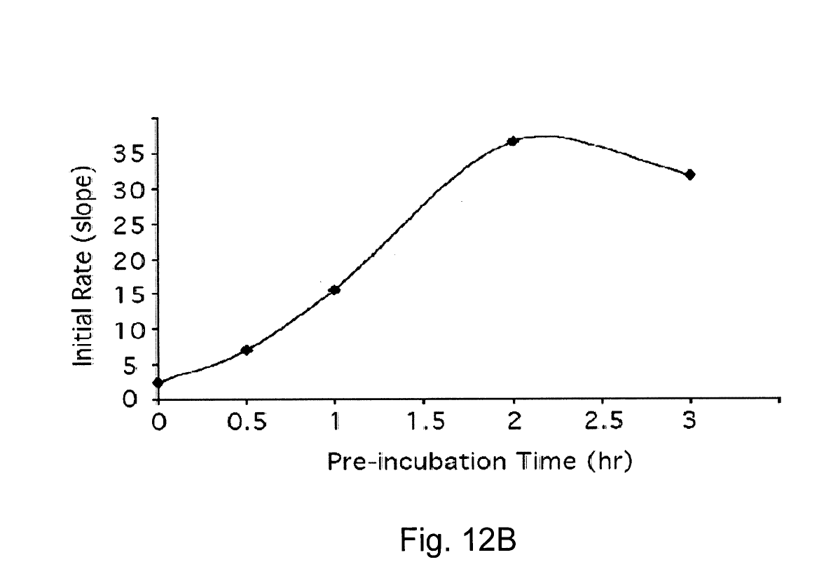

Tyrosine kinase activity is often regulated by autophosphorylation. Single fixed time point assays typically do not reveal whether the kinase is in its fully activated state. By contrast, the pyrene-peptide assay exposed a significant initial lag period in the progress curve for the Brk-catalyzed phosphorylation of pyrene-peptide 23, which was initiated via the addition of ATP (FIG. 12 Panel A, curve a). This observation is consistent with a report by Qiu and Miller, who established that Brk autophosphorylation enhances enzymatic activity (Qiu and Miller (2002) “Regulation of the nonreceptor tyrosine kinase Brk by autophosphorylation and by autoinhibition” J. Biol. Chem. 277:34634-41). By contrast, preincubation of Brk with ATP to ensure full enzyme activation, followed by addition of the pyrene-peptide substrate, furnished a reaction progress curve in which the lag phase is absent (FIG. 12 Panel A, curve b).

FIG. 12 Panel B shows initial phosphorylation rate versus pre-incubation (30° C.) time of Brk and ATP. Brk and ATP were pre-incubated for various time periods (50 mM Tris, 2.5 mM MgCl₂, 1 mM MnCl₂, 2 mM DTT, 1 mM ATP, and 30 nM Brk at pH 7.2), followed by addition of pyrene-peptide 23. The initial rate was subsequently determined and plotted versus pre-incubation time. Maximal enzymatic activity is observed following pre-incubation of Brk with ATP for 2 hr. The subsequently observed reaction progress curve (initiated by the addition of peptide 23) did not display an initial lag phase, suggesting that the enzyme is in a fully activated state. The drop in initial rate at the 3 hr pre-incubation time point is presumably a consequence of a loss in enzymatic activity following extended exposure to 30° C. These results demonstrate that critical features hidden in discontinuous assays are readily revealed using the pyrene-based kinase reporters.

In summary, this example presents a series of exemplary peptides that recognize and signal their phosphorylation status. These species are easily prepared in large quantities, can be modified with unnatural substituents to enhance potency and selectivity (Lee et al. (2004) “A highly potent and selective PKCa inhibitor generated via combinatorial modification of a peptide scaffold” J. Am. Chem. Soc. 126:3394-5), and can be caged at the site of phosphorylation (e.g., with 2-nitrobenzyl as described above; see also, e.g., Veldhuyzen et al. (2003) “A light-activated probe of intracellular protein kinase activity” J. Am. Chem. Soc. 125:13358-9), which enables the investigator to control when the reporter is active. It will be evident that pyrene is used by way of example only; a variety of other fluorophores can noncovalently associate with tyrosine residues and subsequently fluorescently report the introduction of a phosphate group, in any of a variety of substrates.

### Experimental Procedures

Synthesis of Peptide Library

The cystamine-substituted TentaGel S COOH resin was prepared as previously described (Lee and Lawrence (1999) “Acquisition of high-affinity) SH2-targeted ligands via a spatially focused library” J Med Chem 42:784-7). The first amino acid, Fmoc-Ala-OH (5 eq.), was attached to the resin using PyBop (5 eq.), HOBt (5 eq.), and DIPEA (10 eq.) in DMF for 2 h at room temperature. After washing (sequentially with DMF, isopropanol and CH₂Cl₂) and drying, the substitution was determined (0.10 mmol/g) and the peptides subsequently synthesized using an Fmoc solid-phase peptide synthesis protocol. The side chains of Glu and Tyr were protected with t-Bu. A peptide library was prepared by sequentially incorporating Dap and Dab at positions Y−2, Y+1, Y+2, Y+3, and Y+4 in the consensus sequence Ac-Glu-Glu-Glu-Ile-Tyr-Gly-Glu-Ile-Glu-Ala (SEQ ID NO:5). The side chain amines of the Dap and Dab residues were protected during peptide synthesis with the acid sensitive 4-methyltrityl group. Following peptide synthesis, 5 mg of each individual peptide-resin in the library was treated with 1% TFA in CH₂Cl₂ to selectively deprotect Dap or Dab. The free amine in each construct was covalently labeled (acylated) with the succinimidyl ester of 1-pyreneacetic acid (2 eq, DIPEA 4 eq, DMF, overnight). The peptides were then treated with 50% TFA in CH₂Cl₂, washed, and detached from the resin with assay buffer (20 mM DTT in Tris buffer, pH 7.5). The resulting ten peptide solutions were directly assayed for their ability to fluorescently report Src kinase activity. Peptides 23-25 were resynthesized on the Rink resin and purified for detailed NMR and enzymatic studies.

NMR Experiments

NMR experiments were performed at 280 K using a Bruker DRX 600 spectrometer equipped with a 5 mm inverse triple resonance probe. ¹H—¹H NOESY, ¹H—¹H DQF-COSY experiments were carried out on 3 mM samples dissolved in either 90% H₂O/1.0% D₂O or 100% D₂O and adjusted to pH 7.5. Experiments on samples in H₂O used excitation sculpting (Shaka and Hwang (1996) “Water Suppression That Works. Excitation Sculpting Using Arbitrary Wave-Forms and Pulsed-Field Gradients” J. Magn. Reson. A 112:275-279) with gradients for water suppression and experiments on samples in D₂O used presaturation of the residual HOD signal. NOESY spectra were collected using a mixing time of 450 ms. Typically, spectra were collected with 2K and 640 points in F2 and F1 respectively, with 32 scans per t₁ point, a recycle delay of 1.3 s and a proton sweep width of 14 ppm with the carrier set to the water resonance. Spectra were processed using NMRPipe (Delaglio et al. (1995) “NMRPipe: a multidimensional spectral processing system based on UNIX pipes: J. Biomol. NMR 6:277-93) with a cosine bell window function and zero filled to yield data sets with 2K and 1K points in F2 and F1 respectively. Proton chemical shifts were referenced to 3-(trimethylsilyl)propionate. Spectra were analyzed using NMRView (Johnson and Blevins (1994) “NMRView: A computer program for the visualization and analysis of NMR data” J. Biomol. NMR 4:603-14).

TABLE 5
NMR assignments for peptide 23 (see Table 7 for aryl/benzyl
assignments).

| Residue | NH | Alpha | Beta | Gamma | Other |
|---|---|---|---|---|---|
| Glu-1 | 8.47 | 4.2 | 1.89, 2.04 | 2.27 | Ac: 1.98 |
| Glu-2 | 8.63 | 4.18 | 1.90, 2.01 | 2.26 | |
| Glu-3 | 8.41 | 4.1 | 1.81, 2.00 | 2.17 | |
| Ile-4 | 7.99 | 3.89 | 1.56 | 0.59, 0.94, | 0.68 |
| | | | | 1.18 | |
| Tyr-5 | 8.08 | 4.21 | 2.36, 2.48 | | |
| Gly-6 | 7.82 | 3.35, 3.47 | | | |
| Glu-7 | 7.96 | 4.04 | 1.70, 1.95 | 2.07 | |
| DapPyr-8 | 8.38 | 4.48 | 3.51, 3.73 | 8.16 | |
| Glu-9 | 8.53 | 4.21 | 1.88, 2.02 | 2.27 | |
| Ala-10 | 8.33 | 4.11 | 1.31 | | —CONH₂: |
| | | | | | 7.06, 7.53 |

TABLE 6
NMR assignments for peptide 24 (see Table 7 for aryl/benzyl
assignments).

| Residue | NH | Alpha | Beta | Gamma | Other |
|---|---|---|---|---|---|
| Glu-1 | 8.46 | 4.21 | 1.87, 2.04 | 2.26 | Ac: 2.05 |
| Glu-2 | 8.61 | 4.21 | 1.87, 2.01 | 2.24 | |
| Glu-3 | 8.44 | 4.1 | 1.81, 2.00 | 2.16 | |
| Ile-4 | 8.15 | 3.94 | 1.58 | 0.60, 0.96, | 0.69 |
| | | | | 1.23 | |
| pTyr-5 | 8.28 | 4.29 | 2.72 | | |
| Gly-6 | 7.89 | 3.22, 3.58 | | | |
| Glu-7 | 7.96 | 4.05 | 1.71, 1.95 | 2.07 | |
| DapPyr-8 | 8.34 | 4.47 | 3.58, 3.72 | 8.16 | |
| Glu-9 | 8.56 | 4.19 | 1.85, 1.00 | 2.2 | |
| Ala-10 | 8.31 | 4.07 | 1.29 | | —CONH₂: |
| | | | | | 7.05, 7.51 |

TABLE 7
NMR assignments of aromatic and benzylic protons for peptides
23 and 24.

| Proton(s) | Peptide 3 | Peptide 4 |
|---|---|---|
| Pyrene B, C | 8.23, 8.25 | 8.34 |
| Pyrene J | 8.07 | 8.13 |
| Pyrene F, G | 8.06, 8.10 | 8.17, 8.19 |
| Pyrene D | 7.97 | 8.15 |
| Pyrene E | 7.85 | 7.98 |
| Pyrene A | 8.11 | 8.27 |
| Pyrene K | 8.13 | 8.27 |
| Pyrene-CH₂ | 4.28 | 4.39 |
| Tyr-3,5 | 6.53 | 7.06 |
| Tyr-2,6 | 6.61 | 6.95 |
| Tyr-CH₂ | 236, 2.48 | 2.72 |

### Enzyme Assays

Tyrosine kinase-catalyzed phosphorylation was initiated by addition of 15 μL of 10 mM ATP to the following solution: 3 μL 0.1 mM peptide stock solution, 3.8 μL 200 mM MgCl₂, 1.5 μL 100 mM MnCl₂, 23.2 μL H₂O, 6 μL 50 mM DTT, 15 μL 0.1 mg/mL BSA, and 7.5 μL 0.03 μM Src in 75 μL Tris buffer solution (pH 7.2). The final concentration for the screening studies was: 10 μM peptide, 15 nM Src, 1 mM ATP in a buffer containing 50 mM Tris, 5 mM MgCl₂ 1 mM MnCl₂, 0.01 mg/mL BSA, 2 mM DTT at pH 7.5. The fluorescence of the solution was monitored on a Photon Technology QM-1 spectrofluorimeter at 30° C. using an excitation wavelength of 343 nm and an emission wavelength of 380 nm. Vₘₐₓ and Kₘ Values were determined following the assay protocol described above with a Perkin Elmer HTS 7000 Bio Assay Reader (Ex 340 nm and Em 405 nm).

### Example 4

### Tyrosine Kinase Sensors

The following sets forth a series of experiments that demonstrate synthesis and use of enzyme sensors (e.g., kinase and phosphatase sensors) including an environmentally sensitive or fluorescent label. As in Example 3 above, the sensors include self-reporting fluorescent substrates and thus do not require the presence of a detection module.

The pyrene-based protein tyrosine kinase peptides 23 and 25 described above furnish large phosphorylation-induced fluorescent changes (4.3-fold and 4.7-fold, respectively). However, the excitation (340 nm) and emission (380 nm) wavelengths of pyrene are less than ideal for certain applications, for example, for cell-based studies in which autofluorescence at wavelengths near the emission wavelength of pyrene can result in background interference, or for caging sensors with caging groups removable by light near the excitation wavelength of pyrene. Accordingly, based upon the structural features exemplified in 23 and 25, a protein tyrosine kinase peptide library was designed and prepared containing a variety of fluorophores positioned on L-2,4-diaminobutanoic acid 22 (Dab) at the Y-2 position and L-2,3-diaminopropionic acid 21 (Dap) at the Y+3 position. These substitution patterns were chosen because, with the pyrene-containing sensors described above, the largest phosphorylation-induced fluorescence changes were observed at these sites and on these specific residues.

Sensors containing one of several fluorophores display significant changes in their fluorescent properties upon Src kinase-catalyzed phosphorylation of the polypeptide. For example, the Cascade Yellow-containing sensor 26 (FIG. 13), which contains the fluorophore positioned at Y-2, exhibits a 2.7-fold enhancement in fluorescence intensity upon phosphorylation. In contrast, the corresponding peptide containing Cascade Yellow positioned at Y+3 (27) furnishes a smaller fluorescence response to phosphorylation. 2,7-difluorofluorescein (Oregon Green™ 488-X) and Cascade Blue™ exhibit 2-fold enhancements when positioned at Y-2 (sensors 28 and 29, respectively); these fluorophores exhibit somewhat more modest changes in fluorescence upon phosphorylation (1.5-1.7 fold) when positioned at Y+3.

The photophysical properties of these three exemplary fluorophores differ from those of pyrene (see, e.g., Table 8). They can thus be used instead of pyrene, for example, in cell-based studies and/or in caged sensors whose caging groups are removable by light near pyrene's excitation wavelength.

Additional sensors having other fluorophores at positions Y-2 or Y+3 were also prepared and examined. See Table 8 and Table 9.

TABLE 8
Fluorescence change observed upon phosphorylation of exemplary
sensors containing various fluorophores on Dap at position Y + 3.
Excitation (λₑₓ) and emission (λₑₘ) wavelengths in nm
of the labels are shown.

| | | | Fluorescence |
|---|---|---|---|
| Fluorophore at Y + 3 | λₑₓ | λₑₘ | change (fold) |
| Cascade Yellow | 400 | 535 | 1.45 |
| Cascade Blue ™ | 400 | 422 | 1.7 |
| | 379 | 422 | 1.7 |
| Oregon Green ™ 488-X | 495 | 520 | 1.5 |
| NBD | 470 | 535 | 1.25 |
| 1-Pyreneacetyl | 340 | 380 | 4.3 |
| 1-Pyrenesulfonyl | 354 | 384 | 2.3 |
| | 354 | 402 | 2.6 |
| 1-Pyrenebutanoyl | 343 | 378 | 3.7 |
| 7-diethylaminocoumarin-3-carboxyl | 430 | 480 | 0.9 |
| | | 478 | 1.0 |
| 5-carboxyfluorescein (5-FAM, SE) | 494 | 527 | 1.4 |
| single isomer | | | |
| Texas Red ™-X mixed isomers | 593 | 612 | 1.0 |
| Marina Blue ™ | 370 | 456 | 1.3 |
| Pacific Blue ™ | 403 | 458 | 1.5 |
| bimane | 396 | 465 | 1.0 |
| 2-Anthracenesulfonyl | 386 | 437 | 3.3 |
| | 370 | 437 | 3.2 |
| Dansyl | 335 | 431 | 1.0 |
| Alexa Fluoro 430 | 438 | 537 | 1.0 |
| PyMPO | 408 | 554 | 1.6 |
| 5-Carboxytetramethylrhodamine (5- | 555 | 581 | 1.01 |
| TAMRA) | | | |
| 6-Carboxytetramethylrhodamine (6- | 555 | 581 | 1.03 |
| TAMRA) | | | |
| BODIPY FL | 500 | 510 | 1.06 |

TABLE 9
Fluorescence change observed upon phosphorylation of exemplary
sensors containing various fluorophores on Dab at position Y − 2.
Excitation (λₑₓ) and emission (λₑₘ) wavelengths in nm
of the labels are shown.

| | | | Fluorescence |
|---|---|---|---|
| Fluorophore at Y − 2 | λₑₓ | λₑₘ | change (fold) |
| Cascade Yellow | 400 | 535 | 2.7 |
| Cascade Blue ™ | 400 | 422 | 2.1 |
| Oregon Green ™ 488-X | 493 | 526 | 1.8 |
| | 471 | 526 | 2.0 |
| NBD | 470 | 535 | 1.7 |
| 1-Pyreneacetyl | 340 | 380 | 4.8 |
| 1-Pyrenesulfonyl | 350 | 383 | 1.5 |
| 1-Pyrenebutanoyl | 342 | 378 | 3.2 |
| 7-diethylaminocoumarin-3-carboxylic | 428 | 478 | 1.1 |
| acid | | | |
| 5-carboxyfluorescein (5-FAM, SE) | 493 | 526 | 1.0 |
| single isomer | | | |
| Texas Red ™-X mixed isomers | 593 | 622 | 1.0 |
| Marina Blue ™ | 368 | 456 | 1.4 |
| Pacific Blue ™ | 403 | 453 | 1.0 |
| bimane | 394 | 465 | 1.4 |
| 2-Anthracenesulfonyl | 386 | 432 | 2.5 |
| Dansyl | 335 | 431 | 1.0 |
| Alexa Fluoro 430 | 438 | 537 | 1.0 |
| PyMPO | 408 | 554 | 1.2 |
| 5-Carboxytetramethylrhodamine (5- | 555 | 581 | 1.02 |
| TAMRA) | | | |
| 6-Carboxytetramethylrhodamine (6- | 555 | 581 | 1.09 |
| TAMRA) | | | |
| BODIPY FL | 500 | 510 | 1.27 |

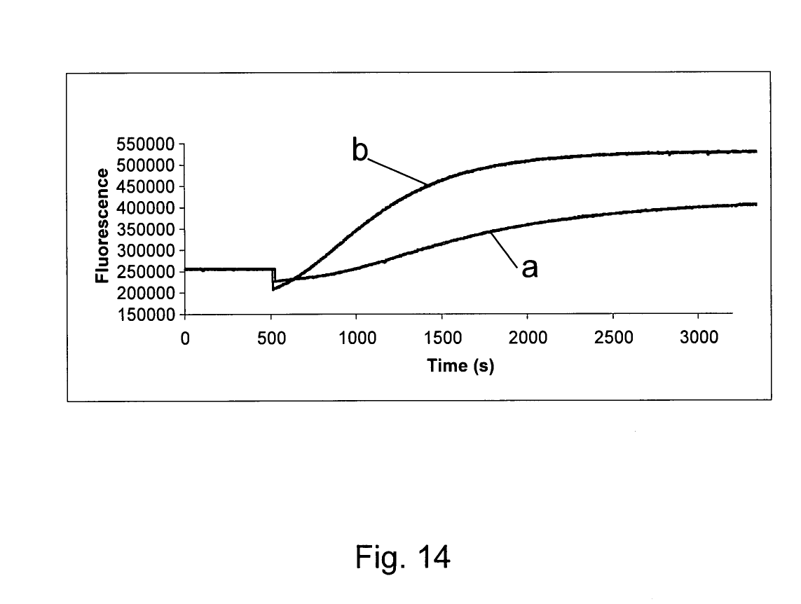

The exemplary sensors are optionally used to detect kinase activity in, for example, samples containing purified kinase, in cell lysates, or in cells. For example, FIG. 14 illustrates detection of Src kinase activity in cell lysates. Sensor 26 was exposed to cell lysate in the absence (curve a) or presence (curve b) of an SH3 ligand (1 mM) that activates Src kinase.

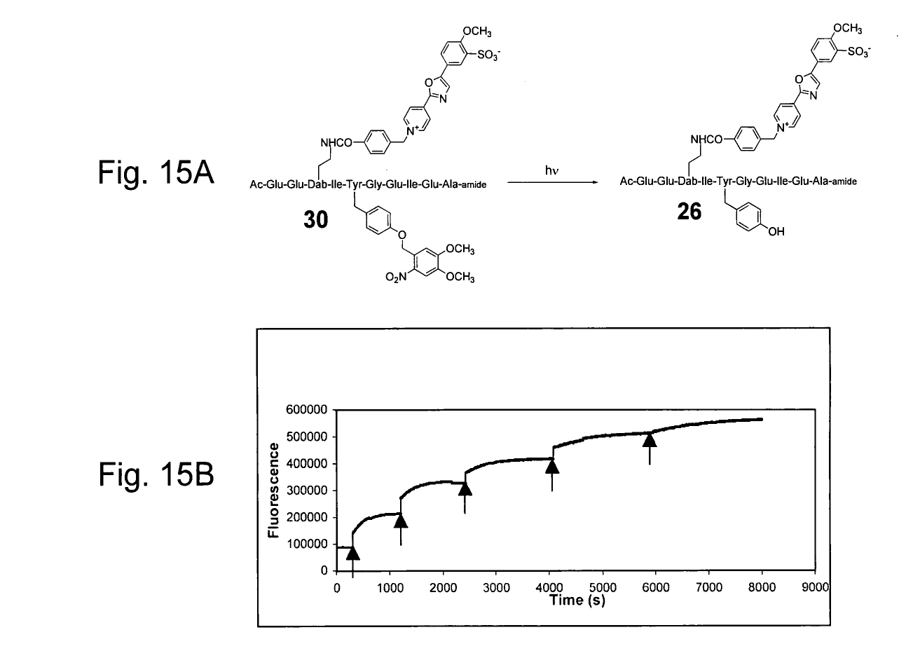

An exemplary caged sensor was produced by covalently attaching a 1-(4,5-dimethoxy-2-nitrophenyl)ethyl (DMNPE) caging group to the tyrosine side chain of Cascade Yellow-containing sensor 26, using standard techniques. The resulting photolabile sensor (30, FIG. 15 Panel A) is inactive and cannot be phosphorylated while the caging group is associated with the polypeptide substrate. The caging group is removed by exposure to light of an appropriate wavelength, liberating active sensor 26.

FIG. 15 Panel B illustrates detection of Src kinase activity in a light dependent manner. Purified Src kinase and caged sensor 30 were introduced into a buffered solution. Well defined amounts of active sensor 26 were liberated (by 8 second exposures to 340-400 nm wavelength light from a filtered mercury arc lamp, exposure marked by arrows in the graph) in a temporally controlled, stepwise fashion. The fluorescent increase levels off at each step once the uncaged amount of the sensor has been completely phosphorylated.

Association of a sensor with a photolabile (or other photoactivatable) caging group thus provides a photochemical switch, permitting a user of the caged sensor to choose when (or where) the sensor is active, providing a technique for sampling kinase activity as a function of temporally (or spatially) sensitive cellular events, such as mitosis, motility, or the like. It will be evident that in some embodiments, a caged sensor preferably includes a fluorophore and a caging group removable by light of a wavelength different from the excitation wavelength of the fluorophore, to avoid undesirable photobleaching of the fluorophore when uncaging the caged sensor.

It will be evident that phosphorylated versions of the above labeled polypeptides are suitable for use as phosphatase sensors. For example, in embodiments in which an increased fluorescent signal is correlated with kinase activity and phosphorylation of the unphosphorylated labeled polypeptide, a decrease in fluorescent signal from the label in the phosphorylated polypeptide is correlated with phosphatase activity and dephosphorylation of the polypeptide.

While the foregoing invention has been described in some detail for purposes of clarity and understanding, it will be clear to one skilled in the art from a reading of this disclosure that various changes in form and detail can be made without departing from the true scope of the invention. For example, all the compositions and techniques described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A composition comprising:
an enzyme, and
a sensor for detecting an activity of the enzyme, the sensor comprising
a) a substrate module comprising
i) a substrate for the enzyme, wherein the substrate is in a first state on which the enzyme can act, thereby converting the substrate to a second state, and
ii) an environmentally sensitive label, and
b) a detection module, which detection module binds to the substrate module when the substrate is in the first state, or which detection module binds to the substrate module when the substrate is in the second state,
wherein binding of the detection module to the substrate module results in a change in signal from the label.

2-36. (canceled)

37. A method of assaying an activity of an enzyme, the method comprising:
contacting the enzyme with a sensor, the sensor comprising
a) a substrate module comprising
i) a substrate for the enzyme, wherein the substrate is in a first state on which the enzyme can act, thereby converting the substrate to a second state, and
ii) an environmentally sensitive label, and
b) a detection module, which detection module binds to the substrate module when the substrate is in the first state, or which detection module binds to the substrate module when the substrate is in the second state,
wherein binding of the detection module to the substrate module results in a change in signal from the label;
detecting the change in signal from the label; and
correlating the change in signal from the label to the activity of the enzyme, thereby assaying the activity of the enzyme.

38-63. (canceled)

64. A composition comprising: a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵;
where X⁻⁴, X⁻³, and X⁻² are independently selected from the group consisting of: D, E, and an amino acid residue comprising the environmentally sensitive or fluorescent label;
where X⁻¹ and X⁺³ are independently selected from the group consisting of: A, V, I, L, M, F, Y, W, and an amino acid residue comprising the environmentally sensitive or fluorescent label;
where X⁺¹, X⁺², X⁺⁴, and X⁺⁵ are independently selected from the group consisting of: an amino acid residue and an amino acid residue comprising the environmentally sensitive or fluorescent label; and
where at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label.

65. The composition of claim 64, wherein one of X⁺¹, X⁺², X⁺³, and X⁺⁴ is an amino acid residue comprising the environmentally sensitive or fluorescent label.

66. The composition of claim 64, wherein the polypeptide comprises an amino acid sequence selected from the group consisting of: EEEIYX⁺¹EIEA (SEQ ID NO:1) where X⁺¹ is an amino acid residue comprising the environmentally sensitive or fluorescent label, EEEIYGX⁺²IEA (SEQ ID NO:2) where X⁺² is an amino acid residue comprising the environmentally sensitive or fluorescent label, EEEIYGEX⁺³ EA (SEQ ID NO:3) where X⁺³ is an amino acid residue comprising the environmentally sensitive or fluorescent label, and EEEIYGEIX⁺⁴A (SEQ ID NO:4) where X⁺⁴ is an amino acid residue comprising the environmentally sensitive or fluorescent label.

67. The composition of claim 66, wherein X⁺¹, X⁺², X⁺³, or X⁺⁴ comprises a Dap, Dab, ornithine, lysine, cysteine, or homocysteine residue.

68. The composition of claim 66, wherein the polypeptide comprises the amino acid sequence EEEIYGEIX⁺⁴A, where X⁺⁴ comprises a dapoxyl group attached to a Dab residue (SEQ ID NO:7); wherein the polypeptide comprises the amino acid sequence EEEIYGEX⁺³EA, where X⁺³ comprises a dapoxyl group attached to a Dab residue (SEQ ID NO:10); or wherein the polypeptide comprises the amino acid sequence EEEIYGEX⁺³EA, where X⁺³ comprises a dapoxyl group attached to a Dap residue (SEQ ID NO:11).

69. The composition of claim 64, wherein one of X⁻² and X⁺³ is an amino acid residue comprising the environmentally sensitive or fluorescent label.

70. The composition of claim 64, wherein the polypeptide comprises an amino acid sequence selected from the group consisting of: EEX⁻²IYGEIEA (SEQ ID NO:9) where X⁻² is an amino acid residue comprising the environmentally sensitive or fluorescent label, and EEEIYGEX⁺³EA (SEQ ID NO:3) where X⁺³ is an amino acid residue comprising the environmentally sensitive or fluorescent label.

71. The composition of claim 70, wherein X⁻² or X⁺³ comprises a Dap, Dab, ornithine, lysine, cysteine, or homocysteine residue.

72. The composition of claim 70, wherein the polypeptide comprises the amino acid sequence EEX⁻²IYGEIEA, where X⁻² comprises pyrene attached to a Dab residue (SEQ ID NO:12); wherein the polypeptide comprises the amino acid sequence EEEIYGEX⁺³EA, where X⁺³ comprises pyrene attached to a Dab residue (SEQ ID NO:13); wherein the polypeptide comprises the amino acid sequence EEEIYGEX⁺³EA, where X⁺³ comprises pyrene attached to a Dap residue (SEQ ID NO:14); wherein the polypeptide comprises the amino acid sequence EEX⁻²IYGEIEA, where X⁻² comprises Cascade Yellow attached to a Dab residue (SEQ ID NO:15); wherein the polypeptide comprises the amino acid sequence EEX⁻²IYGEIEA, where X⁻² comprises 2,7-difluorofluorescein attached to a Dab residue (SEQ ID NO:17); or wherein the polypeptide comprises the amino acid sequence EEEIYGEX⁺³EA, where X⁺³ comprises 2,7-difluorofluorescein attached to a Dap residue (SEQ ID NO:18).

73. The composition of claim 70, wherein the label comprises 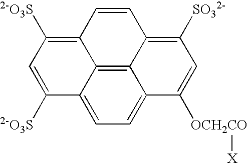
where X represents the site of attachment to the polypeptide; and
wherein the polypeptide comprises the amino acid sequence EEX⁻²IYGEIEA, where X⁻² comprises the label attached to a Dab residue (SEQ ID NO:19) or the amino acid sequence EEEIYGEX⁺³EA, where X⁺³ comprises the label attached to a Dap residue (SEQ ID NO:20).

74. The composition of claim 64, wherein the label is a fluorescent label.

75. The composition of claim 64, wherein the label comprises a fluorophore selected from the group consisting of: 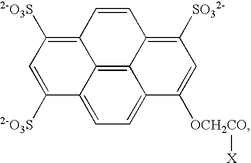 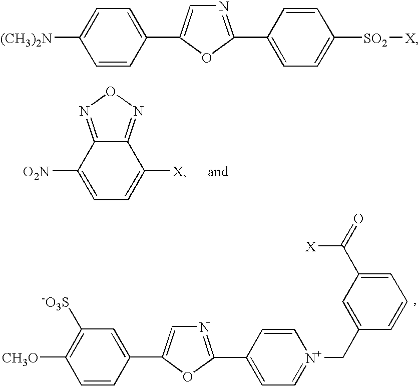
where X represents the site of attachment to the polypeptide.

76. The composition of claim 64, wherein the label comprises pyrene or 2,7-difluorofluorescein.

77. The composition of claim 64, wherein the label comprises a label selected from the group consisting of: 7-diethylaminocoumarin-3-carboxylic acid, 5-carboxyfluorescein, bimane, 2-anthracenesulfonyl, dansyl, Alexa Fluor 430, PyMPO, 5-carboxytetramethylrhodamine (5-TAMRA), 6-carboxytetramethylrhodamine (6-TAMRA), BODIPY FL, and 3,4,9,10-perylene-tetracarboxylic acid.

78. The composition of claim 64, comprising a tyrosine protein kinase.

79. The composition of claim 78, wherein the kinase is selected from the group consisting of: Src, SrcN1, SrcN2, FynT, Fgr, Lck, Yes, LynA, LynB, Hck, Abl, Csk, Fes/Fps, FGFR, TrkA, and Flt3.

80. The composition of claim 64, wherein Y⁰ comprises a free hydroxyl group.

81. The composition of claim 64, wherein Y⁰ is a phosphorylated tyrosine residue.

82. The composition of claim 64, comprising a protein phosphatase.

83. The composition of claim 64, further comprising a second polypeptide comprising an SH2 domain, a PTB domain, or an antibody.

84. The composition of claim 64, wherein phosphorylation of Y⁰ results in a change in signal from the label.

85. The composition of claim 84, wherein the label is a fluorescent label, and wherein the change in signal from the label is a change in fluorescence emission intensity.

86. The composition of claim 85, wherein the change in signal from the label is a change of greater than ±25%, greater than ±50%, greater than ±75%, greater than ±90%, greater than ±95%, greater than ±98%, greater than +100%, greater than +200%, greater than +300%, greater than +400%, greater than +500%, greater than +600%, or greater than +700% in fluorescence emission intensity.

87. The composition of claim 64, comprising a cell or a cell lysate.

88. The composition of claim 64, wherein the composition comprises one or more caging groups, which caging groups are associated with the polypeptide, and which caging groups inhibit an enzyme from acting upon the polypeptide.

89. The composition of claim 88, wherein the one or more caging groups inhibit the enzyme from acting upon the polypeptide by at least about 75%, at least about 90%, at least about 95%, or at least about 98%, as compared to the polypeptide in the absence of the one or more caging groups.

90. The composition of claim 88, wherein the one or more caging groups prevent the enzyme from acting upon the polypeptide.

91. The composition of claim 88, wherein the one or more caging groups associated with the polypeptide are covalently attached to the polypeptide.

92. The composition of claim 91, wherein the composition comprises a single caging group, which caging group is covalently attached to the Y⁰ side chain.

93. The composition of claim 88, wherein the one or more caging groups are photoactivatable or photolabile.

94. A composition comprising:
a polypeptide comprising an environmentally sensitive or fluorescent label, which polypeptide comprises a tyrosine residue;
wherein when the tyrosine is unphosphorylated it engages in an interaction with the label, which interaction is at least partially disrupted when the tyrosine is phosphorylated;
whereby a signal from the label changes upon phosphorylation or dephosphorylation of the tyrosine.

95-109. (canceled)

110. A composition comprising: a polypeptide substrate for a protein tyrosine kinase or a tyrosine-specific protein phosphatase, which polypeptide substrate comprises an environmentally sensitive or fluorescent label, wherein the environmentally sensitive or fluorescent label is located at amino acid position −2 or +3 with respect to the phosphorylation site within the polypeptide substrate.

111-121. (canceled)

122. A method of assaying an activity of an enzyme, the method comprising:
contacting the enzyme with a sensor, the sensor comprising:
a) a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵,
where X⁻⁴, X⁻³, and X⁻² are independently selected from the group consisting of: D, E, and an amino acid residue comprising the environmentally sensitive or fluorescent label,
where X⁻¹ and X⁺³ are independently selected from the group consisting of: A, V, I, L, M, F, Y, W, and an amino acid residue comprising the environmentally sensitive or fluorescent label,
where X⁺¹, X⁺², X⁺⁴, and X⁺⁵ are independently selected from the group consisting of: an amino acid residue and an amino acid residue comprising the environmentally sensitive or fluorescent label, and
where at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label,
wherein phosphorylation or dephosphorylation of Y⁰ results in a change in signal from the label,
b) a polypeptide comprising an environmentally sensitive or fluorescent label, which polypeptide comprises a tyrosine residue,
wherein when the tyrosine is unphosphorylated it engages in an interaction with the label, which interaction is at least partially disrupted when the tyrosine is phosphorylated,
whereby a signal from the label changes upon phosphorylation or dephosphorylation of the tyrosine, or
c) a polypeptide substrate for a protein tyrosine kinase or a tyrosine-specific protein phosphatase, which polypeptide substrate comprises an environmentally sensitive or fluorescent label, wherein the environmentally sensitive or fluorescent label is located at amino acid position −2 or +3 with respect to the phosphorylation site within the polypeptide substrate,
wherein phosphorylation or dephosphorylation of the substrate at the phosphorylation site results in a change in signal from the label;
detecting the change in signal from the label; and
correlating the change in signal from the label to the activity of the enzyme, thereby assaying the activity of the enzyme.

123-131. (canceled)

132. A method of determining whether a test compound affects an activity of an enzyme, the method comprising:
providing a cell comprising the enzyme;
introducing a sensor into the cell, the sensor comprising:
a) a polypeptide comprising an environmentally sensitive or fluorescent label, which polypeptide comprises amino acid sequence X⁻⁴ X⁻³ X⁻² X⁻¹ Y⁰ X⁺¹ X⁺² X⁺³ X⁺⁴ X⁺⁵,
where X⁻⁴, X⁻³, and X⁻² are independently selected from the group consisting of: D, E, and an amino acid residue comprising the environmentally sensitive or fluorescent label,
where X⁻¹ and X⁺³ are independently selected from the group consisting of: A, V, I, L, M, F, Y, W, and an amino acid residue comprising the environmentally sensitive or fluorescent label,
where X⁺¹, X⁺², X⁺⁴, and X⁺⁵ are independently selected from the group consisting of: an amino acid residue and an amino acid residue comprising the environmentally sensitive or fluorescent label, and
where at least one of X⁻⁴, X⁻³, X⁻², X⁻¹, X⁺¹, X⁺², X⁺³, X⁺⁴, and X⁺⁵ is an amino acid residue comprising the environmentally sensitive or fluorescent label,
wherein phosphorylation or dephosphorylation of Y⁰ results in a change in signal from the label;
b) a polypeptide comprising an environmentally sensitive or fluorescent label, which polypeptide comprises a tyrosine residue,
wherein when the tyrosine is unphosphorylated it engages in an interaction with the label, which interaction is at least partially disrupted when the tyrosine is phosphorylated,
whereby a signal from the label changes upon phosphorylation or dephosphorylation of the tyrosine;
c) a polypeptide substrate for a protein tyrosine kinase or a tyrosine-specific protein phosphatase, which polypeptide substrate comprises an environmentally sensitive or fluorescent label, wherein the environmentally sensitive or fluorescent label is located at amino acid position −2 or +3 with respect to the phosphorylation site within the polypeptide substrate, wherein phosphorylation or dephosphorylation of the substrate at the phosphorylation site results in a change in signal from the label; or
d) i) a substrate module comprising
1) a substrate for the enzyme, wherein the substrate is in a first state on which the enzyme can act, thereby converting the substrate to a second state, and
2) an environmentally sensitive label, and
ii) a detection module, which detection module binds to the substrate module when the substrate is in the first state, or which detection module binds to the substrate module when the substrate is in the second state,
wherein binding of the detection module to the substrate module results in a change in signal from the label;
contacting the cell with the test compound; and
detecting the change in signal from the label, the change in signal providing an indication of the activity of the enzyme in the presence of the test compound.

133-136. (canceled)


SEQUENCE LISTING (159 sequences)

SEQ ID NO: 1 - protein, 10 aa, Artificial sequence
  synthetic peptide substrate
EEEIYXEIEA

SEQ ID NO: 2 - protein, 10 aa, Artificial sequence
  synthetic peptide substrate
EEEIYGXIEA

SEQ ID NO: 3 - protein, 10 aa, Artificial sequence
  synthetic peptide substrate
EEEIYGEXEA

SEQ ID NO: 4 - protein, 10 aa, Artificial sequence
  synthetic peptide substrate
EEEIYGEIXA

SEQ ID NO: 5 - protein, 10 aa, Artificial sequence
  synthetic peptide substrate
EEEIYGEIEA

SEQ ID NO: 6 - protein, 10 aa, Artificial sequence
  synthetic peptide substrate
EEEIYXEIEA

SEQ ID NO: 7 - protein, 10 aa, Artificial sequence
  synthetic peptide substrate
EEEIYGEIXA

SEQ ID NO: 8 - protein, 5 aa, Artificial sequence
  Lck SH2 domain ligand
YEEIE

SEQ ID NO: 9 - protein, 10 aa, Artificial sequence
  synthetic peptide substrate
EEXIYGEIEA

SEQ ID NO: 10 - protein, 10 aa, Artificial sequence
  synthetic peptide substrate
EEEIYGEXEA

SEQ ID NO: 11 - protein, 10 aa, Artificial sequence
  synthetic peptide substrate
EEEIYGEXEA

SEQ ID NO: 12 - protein, 10 aa, Artificial sequence
  synthetic peptide substrate
EEXIYGEIEA

SEQ ID NO: 13 - protein, 10 aa, Artificial sequence
  synthetic peptide substrate
EEEIYGEXEA

SEQ ID NO: 14 - protein, 10 aa, Artificial sequence
  synthetic peptide substrate
EEEIYGEXEA

SEQ ID NO: 15 - protein, 10 aa, Artificial sequence
  synthetic peptide substrate
EEXIYGEIEA

SEQ ID NO: 16 - protein, 10 aa, Artificial sequence
  synthetic peptide substrate
EEEIYGEXEA

SEQ ID NO: 17 - protein, 10 aa, Artificial sequence
  synthetic peptide substrate
EEXIYGEIEA

SEQ ID NO: 18 - protein, 10 aa, Artificial sequence
  synthetic peptide substrate
EEEIYGEXEA

SEQ ID NO: 19 - protein, 10 aa, Artificial sequence
  synthetic peptide substrate
EEXIYGEIEA

SEQ ID NO: 20 - protein, 10 aa, Artificial sequence
  synthetic peptide substrate
EEEIYGEXEA

SEQ ID NO: 21 - protein, 12 aa, Artificial sequence
  synthetic peptide substrate
LLDKYLIPNATQ

SEQ ID NO: 22 - protein, 12 aa, Artificial sequence
  synthetic peptide substrate
YEILNSPEKACS

SEQ ID NO: 23 - protein, 13 aa, Artificial sequence
  synthetic peptide substrate
LTLKKTPGRSTGE

SEQ ID NO: 24 - protein, 12 aa, Artificial sequence
  synthetic peptide substrate
VNPYYLRVRRKN

SEQ ID NO: 25 - protein, 13 aa, Artificial sequence
  synthetic peptide substrate
HGGHKTPRRDSSG

SEQ ID NO: 26 - protein, 13 aa, Artificial sequence
  synthetic peptide substrate
LTPEKSPKFPDSQ

SEQ ID NO: 27 - protein, 14 aa, Artificial sequence
  synthetic peptide substrate
SGGLELYGEPRHTT

SEQ ID NO: 28 - protein, 13 aa, Artificial sequence
  synthetic peptide substrate
MHSVYQPQPSASQ

SEQ ID NO: 29 - protein, 12 aa, Artificial sequence
  synthetic peptide substrate
LWEAYANLHTAV

SEQ ID NO: 30 - protein, 13 aa, Artificial sequence
  synthetic peptide substrate
RSNPKSPQKPIVR

SEQ ID NO: 31 - protein, 13 aa, Artificial sequence
  synthetic peptide substrate
LRRDKSPGRPLER

SEQ ID NO: 32 - protein, 13 aa, Artificial sequence
  synthetic peptide substrate
LEREKSPGRMLST

SEQ ID NO: 33 - protein, 14 aa, Artificial sequence
  synthetic peptide substrate
DSTAETYGKIVHYK

SEQ ID NO: 34 - protein, 13 aa, Artificial sequence
  synthetic peptide substrate
KAEEKSPKKQKVT

SEQ ID NO: 35 - protein, 13 aa, Artificial sequence
  synthetic peptide substrate
TVWKKSPEKNERH

SEQ ID NO: 36 - protein, 12 aa, Artificial sequence
  synthetic peptide substrate
EEMTYEEIQEHY

SEQ ID NO: 37 - protein, 12 aa, Artificial sequence
  synthetic peptide substrate
VESIYLNVEAVN

SEQ ID NO: 38 - protein, 12 aa, Artificial sequence
  synthetic peptide substrate
EELTYEEIRDTY

SEQ ID NO: 39 - protein, 12 aa, Artificial sequence
  synthetic peptide substrate
EEMTYEEIQDNY

SEQ ID NO: 40 - protein, 12 aa, Artificial sequence
  synthetic peptide substrate
PPEEYVPMVKEV

SEQ ID NO: 41 - protein, 14 aa, Artificial sequence
  synthetic peptide substrate
FSSSEIYGLIKTGA

SEQ ID NO: 42 - protein, 12 aa, Artificial sequence
  synthetic peptide substrate
GTVGYMAPEVVK

SEQ ID NO: 43 - protein, 12 aa, Artificial sequence
  synthetic peptide substrate
QKYAYLNVVGMV

SEQ ID NO: 44 - protein, 14 aa, Artificial sequence
  synthetic peptide substrate
LGTEELYGYLKKYH

SEQ ID NO: 45 - protein, 14 aa, Artificial sequence
  synthetic peptide substrate
VLRKEAYGKPVDIW

SEQ ID NO: 46 - protein, 12 aa, Artificial sequence
  synthetic peptide substrate
MFMWYLNPRQVF

SEQ ID NO: 47 - protein, 12 aa, Artificial sequence
  synthetic peptide substrate
KDEVYLNLVLDY

SEQ ID NO: 48 - protein, 14 aa, Artificial sequence
  synthetic peptide substrate
ELLTELYGKVGEIR

SEQ ID NO: 49 - protein, 13 aa, Artificial sequence
  synthetic peptide substrate
RDGYKTPKEDPNR

SEQ ID NO: 50 - protein, 14 aa, Artificial sequence
  synthetic peptide substrate
NLLGELYGKAGLNQ

SEQ ID NO: 51 - protein, 13 aa, Artificial sequence
  synthetic peptide substrate
RDGYKTPREDPNR

SEQ ID NO: 52 - protein, 12 aa, Artificial sequence
  synthetic peptide substrate
EGFSYVNPQFVH

SEQ ID NO: 53 - protein, 13 aa, Artificial sequence
  synthetic peptide substrate
RPKVKSPRDYSNF

SEQ ID NO: 54 - protein, 12 aa, Artificial sequence
  synthetic peptide substrate
KFNGYLRVRIGE

SEQ ID NO: 55 - protein, 12 aa, Artificial sequence
  synthetic peptide substrate
VWVDYPNIVRVV

SEQ ID NO: 56 - protein, 12 aa, Artificial sequence
  synthetic peptide substrate
GTAAYMAPEVIT

SEQ ID NO: 57 - protein, 12 aa, Artificial sequence
  synthetic peptide substrate
GTLQYMAPEIID

SEQ ID NO: 58 - protein, 14 aa, Artificial sequence
  synthetic peptide substrate
ENIAELYGAVLWGE

SEQ ID NO: 59 - protein, 13 aa, Artificial sequence
  synthetic peptide substrate
PNLGKSPKHTPIA

SEQ ID NO: 60 - protein, 13 aa, Artificial sequence
  synthetic peptide substrate
VGGLKSPWRGEYK

SEQ ID NO: 61 - protein, 13 aa, Artificial sequence
  synthetic peptide substrate
VTLTKSPKKRPSA

SEQ ID NO: 62 - protein, 13 aa, Artificial sequence
  synthetic peptide substrate
LQHPYINVWYDPA

SEQ ID NO: 63 - protein, 12 aa, Artificial sequence
  synthetic peptide substrate
GTRSYMAPERLQ

SEQ ID NO: 64 - protein, 12 aa, Artificial sequence
  synthetic peptide substrate
GCRPYMAPERID

SEQ ID NO: 65 - protein, 12 aa, Artificial sequence
  synthetic peptide substrate
GTNAYMAPERIS

SEQ ID NO: 66 - protein, 12 aa, Artificial sequence
  synthetic peptide substrate
GCKPYMAPERIN

SEQ ID NO: 67 - protein, 12 aa, Artificial sequence
  synthetic peptide substrate
GCAAYMAPERID

SEQ ID NO: 68 - protein, 12 aa, Artificial sequence
  synthetic peptide substrate
AAYCYLRVVGKG

SEQ ID NO: 69 - protein, 12 aa, Artificial sequence
  synthetic peptide substrate
GDPRYMAPELLQ

SEQ ID NO: 70 - protein, 13 aa, Artificial sequence
  synthetic peptide substrate
PVPKKSPKSQPLE

SEQ ID NO: 71 - protein, 12 aa, Artificial sequence
  synthetic peptide substrate
SEDVYTAVEHSD

SEQ ID NO: 72 - protein, 14 aa, Artificial sequence
  synthetic peptide substrate
QWFREAYGAVTQTV

SEQ ID NO: 73 - protein, 13 aa, Artificial sequence
  synthetic peptide substrate
LTWNKSPKSVLVI

SEQ ID NO: 74 - protein, 12 aa, Artificial sequence
  synthetic peptide substrate
MSPDYPNPMFEH

SEQ ID NO: 75 - protein, 12 aa, Artificial sequence
  synthetic peptide substrate
KAAGYANPVWTA

SEQ ID NO: 76 - protein, 14 aa, Artificial sequence
  synthetic peptide substrate
QRSAKSPRREEEPR

SEQ ID NO: 77 - protein, 14 aa, Artificial sequence
  synthetic peptide substrate
ESLVETYGKIMNHK

SEQ ID NO: 78 - protein, 14 aa, Artificial sequence
  synthetic peptide substrate
ESLVETYGKIMNHE

SEQ ID NO: 79 - protein, 12 aa, Artificial sequence
  synthetic peptide substrate
GTKPYMAPEVFQ

SEQ ID NO: 80 - protein, 13 aa, Artificial sequence
  synthetic peptide substrate
LVRSKSPKITYFT

SEQ ID NO: 81 - protein, 12 aa, Artificial sequence
  synthetic peptide substrate
GSPMYMAPEVIM

SEQ ID NO: 82 - protein, 12 aa, Artificial sequence
  synthetic peptide substrate
EGFEYINPLLMS

SEQ ID NO: 83 - protein, 12 aa, Artificial sequence
  synthetic peptide substrate
GLQNYLNVITTN

SEQ ID NO: 84 - protein, 12 aa, Artificial sequence
  synthetic peptide substrate
DGNGYISAAELR

SEQ ID NO: 85 - protein, 12 aa, Artificial sequence
  synthetic peptide substrate
YAIKYVNLEEAD

SEQ ID NO: 86 - protein, 12 aa, Artificial sequence
  synthetic peptide substrate
YGDIYLNAGPML

SEQ ID NO: 87 - protein, 12 aa, Artificial sequence
  synthetic peptide substrate
KWKMYMEMDGDE

SEQ ID NO: 88 - protein, 13 aa, Artificial sequence
  synthetic peptide substrate
ISNFKTPSKLSEK

SEQ ID NO: 89 - protein, 14 aa, Artificial sequence
  synthetic peptide substrate
PKSEEPYGQLNPKW

SEQ ID NO: 90 - protein, 13 aa, Artificial sequence
  synthetic peptide substrate
RSIIKTPKTQDTE

SEQ ID NO: 91 - protein, 14 aa, Artificial sequence
  synthetic peptide substrate
SGRLKTPGKREIPV

SEQ ID NO: 92 - protein, 12 aa, Artificial sequence
  synthetic peptide substrate
GSPLYMAPEMVC

SEQ ID NO: 93 - protein, 12 aa, Artificial sequence
  synthetic peptide substrate
GTLYYMAPEHLN

SEQ ID NO: 94 - protein, 12 aa, Artificial sequence
  synthetic peptide substrate
NQETYLNISQVN

SEQ ID NO: 95 - protein, 13 aa, Artificial sequence
  synthetic peptide substrate
PHNPKTPPKSPVV

SEQ ID NO: 96 - protein, 13 aa, Artificial sequence
  synthetic peptide substrate
LTHDYINLFHFPG

SEQ ID NO: 97 - protein, 13 aa, Artificial sequence
  synthetic peptide substrate
PANQKSPKGKLRL

SEQ ID NO: 98 - protein, 13 aa, Artificial sequence
  synthetic peptide substrate
CENAKTPKEQFRV

SEQ ID NO: 99 - protein, 12 aa, Artificial sequence
  synthetic peptide substrate
REKEYVNIQTFR

SEQ ID NO: 100 - protein, 12 aa, Artificial sequence
  synthetic peptide substrate
LAKWYVNAKGYF

SEQ ID NO: 101 - protein, 12 aa, Artificial sequence
  synthetic peptide substrate
PDDKYIYVADIL

SEQ ID NO: 102 - protein, 12 aa, Artificial sequence
  synthetic peptide substrate
LRFLESPDFQPS

SEQ ID NO: 103 - protein, 12 aa, Artificial sequence
  synthetic peptide substrate
LPPASTPTSPSS

SEQ ID NO: 104 - protein, 12 aa, Artificial sequence
  synthetic peptide substrate
ASTPTSPSSPGL

SEQ ID NO: 105 - protein, 12 aa, Artificial sequence
  synthetic peptide substrate
PTSPSSPGLSPV

SEQ ID NO: 106 - protein, 12 aa, Artificial sequence
  synthetic peptide substrate
SSPGLSPVPPPD

SEQ ID NO: 107 - protein, 12 aa, Artificial sequence
  synthetic peptide substrate
NQQELTPLPLLK

SEQ ID NO: 108 - protein, 12 aa, Artificial sequence
  synthetic peptide substrate
ARCVSSPHFQVA

SEQ ID NO: 109 - protein, 12 aa, Artificial sequence
  synthetic peptide substrate
PLQRLTPQVAAS

SEQ ID NO: 110 - protein, 12 aa, Artificial sequence
  synthetic peptide substrate
ISHEHSPSDLEA

SEQ ID NO: 111 - protein, 12 aa, Artificial sequence
  synthetic peptide substrate
VIMGLSPILGKD

SEQ ID NO: 112 - protein, 12 aa, Artificial sequence
  synthetic peptide substrate
LCSDDTPMVRRA

SEQ ID NO: 113 - protein, 12 aa, Artificial sequence
  synthetic peptide substrate
ISQEHTPVALEA

SEQ ID NO: 114 - protein, 12 aa, Artificial sequence
  synthetic peptide substrate
QLTPFSPVFGTE

SEQ ID NO: 115 - protein, 12 aa, Artificial sequence
  synthetic peptide substrate
LKKCPTPMQNEI

SEQ ID NO: 116 - protein, 12 aa, Artificial sequence
  synthetic peptide substrate
KSKVSSPIEKVS

SEQ ID NO: 117 - protein, 12 aa, Artificial sequence
  synthetic peptide substrate
PIEKVSPSCLTR

SEQ ID NO: 118 - protein, 12 aa, Artificial sequence
  synthetic peptide substrate
LSVCRSPVGDKA

SEQ ID NO: 119 - protein, 12 aa, Artificial sequence
  synthetic peptide substrate
VLIQQTPEVIKI

SEQ ID NO: 120 - protein, 12 aa, Artificial sequence
  synthetic peptide substrate
EKKPGTPLPPPA

SEQ ID NO: 121 - protein, 12 aa, Artificial sequence
  synthetic peptide substrate
SESAYPNAELVF

SEQ ID NO: 122 - protein, 12 aa, Artificial sequence
  synthetic peptide substrate
KEIVYPNIEETH

SEQ ID NO: 123 - protein, 13 aa, Artificial sequence
  synthetic peptide substrate
AELIKTPRVDNVV

SEQ ID NO: 124 - protein, 12 aa, Artificial sequence
  synthetic peptide substrate
LTYIYPNIIAMG

SEQ ID NO: 125 - protein, 12 aa, Artificial sequence
  synthetic peptide substrate
EDNDYINASLIK

SEQ ID NO: 126 - protein, 12 aa, Artificial sequence
  synthetic peptide substrate
AESCYINIARTL

SEQ ID NO: 127 - protein, 12 aa, Artificial sequence
  synthetic peptide substrate
GNEDYINANYIN

SEQ ID NO: 128 - protein, 13 aa, Artificial sequence
  synthetic peptide substrate
WPDQKTPDRAPPL

SEQ ID NO: 129 - protein, 12 aa, Artificial sequence
  synthetic peptide substrate
PGSDYINANYIK

SEQ ID NO: 130 - protein, 12 aa, Artificial sequence
  synthetic peptide substrate
EDGDYINANYIR

SEQ ID NO: 131 - protein, 13 aa, Artificial sequence
  synthetic peptide substrate
PPPEKTPAKKHVR

SEQ ID NO: 132 - protein, 12 aa, Artificial sequence
  synthetic peptide substrate
TQTDYINASFMD

SEQ ID NO: 133 - protein, 12 aa, Artificial sequence
  synthetic peptide substrate
KGHEYTNIKYSL

SEQ ID NO: 134 - protein, 12 aa, Artificial sequence
  synthetic peptide substrate
SARVYENVGLMQ

SEQ ID NO: 135 - protein, 12 aa, Artificial sequence
  synthetic peptide substrate
QDSDYINANFIK

SEQ ID NO: 136 - protein, 12 aa, Artificial sequence
  synthetic peptide substrate
DEGGYINASFIK

SEQ ID NO: 137 - protein, 13 aa, Artificial sequence
  synthetic peptide substrate
KKQCKSPSRRDSY

SEQ ID NO: 138 - protein, 12 aa, Artificial sequence
  synthetic peptide substrate
LFPIYENVNPEY

SEQ ID NO: 139 - protein, 12 aa, Artificial sequence
  synthetic peptide substrate
ARSDYLRVSWVH

SEQ ID NO: 140 - protein, 13 aa, Artificial sequence
  synthetic peptide substrate
PCSDYINASYIPG

SEQ ID NO: 141 - protein, 12 aa, Artificial sequence
  synthetic peptide substrate
IKGYYIIIVPLK

SEQ ID NO: 142 - protein, 12 aa, Artificial sequence
  synthetic peptide substrate
YSIKYTAVDGED

SEQ ID NO: 143 - protein, 12 aa, Artificial sequence
  synthetic peptide substrate
EKNRYPNILPND

SEQ ID NO: 144 - protein, 12 aa, Artificial sequence
  synthetic peptide substrate
EYTDYINASFID

SEQ ID NO: 145 - protein, 12 aa, Artificial sequence
  synthetic peptide substrate
KHSDYINANYVD

SEQ ID NO: 146 - protein, 12 aa, Artificial sequence
  synthetic peptide substrate
SRSDYINASPII

SEQ ID NO: 147 - protein, 12 aa, Artificial sequence
  synthetic peptide substrate
SHSDYINASPIM

SEQ ID NO: 148 - protein, 12 aa, Artificial sequence
  synthetic peptide substrate
HKNRYINIVAYD

SEQ ID NO: 149 - protein, 12 aa, Artificial sequence
  synthetic peptide substrate
KLTDYINANYVD

SEQ ID NO: 150 - protein, 13 aa, Artificial sequence
  synthetic peptide substrate
HIHAYVNALLIPG

SEQ ID NO: 151 - protein, 12 aa, Artificial sequence
  synthetic peptide substrate
EGTDYINASYIM

SEQ ID NO: 152 - protein, 12 aa, Artificial sequence
  synthetic peptide substrate
GKDDYINASCVE

SEQ ID NO: 153 - protein, 13 aa, Artificial sequence
  synthetic peptide substrate
SYNEKTPRIVVSR

SEQ ID NO: 154 - protein, 12 aa, Artificial sequence
  synthetic peptide substrate
ALVQYINQLCRH

SEQ ID NO: 155 - protein, 12 aa, Artificial sequence
  synthetic peptide substrate
CGLPYINLEFLK

SEQ ID NO: 156 - protein, 13 aa, Artificial sequence
  synthetic peptide substrate
TVKPKSPEKSKPD

SEQ ID NO: 157 - protein, 13 aa, Artificial sequence
  synthetic peptide substrate
KDPEKSPTKKQEV

SEQ ID NO: 158 - protein, 12 aa, Artificial sequence
  synthetic peptide substrate
EDSSYINANFIK

SEQ ID NO: 159 - protein, 13 aa, Artificial sequence
  synthetic peptide substrate
KQTLKTPGKSFTR